SEQUENCE LISTING (7 sequences)

SEQ ID NO: 1 - DNA, 24 bp, Artificial Sequence
  Synthetic polynucleotide
ccaaataaaaggaagtgaaaccaa

SEQ ID NO: 2 - DNA, 52 bp, Artificial Sequence
  Synthetic polynucleotide
ccaaataaaaggaagtgaaaccaagcccaaataaaaggaagtgaaaccaagc

SEQ ID NO: 3 - DNA, 54 bp, Artificial Sequence
  Synthetic polynucleotide
  misc_feature (1)..(15): n may be a, c, g, or t
  misc_feature (6)..(15): may be absent
  misc_feature (20)..(54): n is a, c, g, or t
  misc_feature (25)..(54): may be absent
nnnnnnnnnnnnnnnggaannnnnnnnnnnnnnnnnnnnnnnnnnnnnnnnnnn

SEQ ID NO: 4 - DNA, 10 bp, Artificial Sequence
  Synthetic polynucleotide
ccaaataaaa

SEQ ID NO: 5 - DNA, 10 bp, Artificial Sequence
  Synthetic polynucleotide
gtgaaaccaa

SEQ ID NO: 6 - DNA, 130 bp, Artificial Sequence
  Synthetic polynucleotide
ccaaataaaaggaagtgaaaccaagcccaaataaaaggaagtgaaaccaagcccaaataaaaggaagtgaaaccaagcccaaataaaaggaagtgaaaccaagcccaaataaaaggaagtgaaaccaagc

SEQ ID NO: 7 - DNA, 24 bp, Artificial Sequence
  Synthetic polynucleotide
ttggtttcacttccttttatttgg

(19) United States
(12) Patent Application Publication
(10) Pub. No.: US 2022/0340983 A1
(43) Pub. Date: Oct. 27, 2022

(54) SYSTEMS AND ASSAYS FOR IDENTIFYING PU.1 INHIBITORS

(71) Applicant: Massachustts Institute of Technology, Cambridge, MA (US)

(72) Inventors: Li-Huei Tsai, Cambridge, MA (US); William Ralvenius, Somerville, MA (US); Alison Mungenast, Arlington, MA (US)

(73) Assignee: Massachustts Institute of Technology, Cambridge, MA (US)

(21) Appl. No.: 17/642,546

(22) PCT Filed: Sep. 11, 2020
(86) PCT No.: PCT/US20/50580

§ 371 Date: Mar. 11, 2022

Related U.S. Application Data
(60) Provisional application No. 62/900,328, filed on Sep. 13, 2019.

(51) Int. Cl. C12Q 1/6897; A61K 31/404; A61K 31/343; A61K 31/416; A61K 31/4184; A61K 31/423; A61K 31/4709

(52) U.S. Cl. CPC C12Q 1/6897; A61K 31/404; A61K 31/343; A61K 31/416; A61K 31/4184; A61K 31/423; A61K 31/4709

(57) ABSTRACT

The disclosure relates to compositions comprising PU.1 inhibitors as well as methods of making and using the same. In some embodiments, methods of screening compounds for PU.1 inhibition are disclosed. In some embodiments, methods of screening a plurality of compounds for PU.1 inhibition are disclosed. In some embodiments, lambda-beta binding (LBB) motifs are used to screen compounds for PU.1 inhibition. In some embodiments, methods of treating neurodegenerative disorders are disclosed. In some embodiments pharmaceutical compounds are provided. In some embodiments, methods of treating Alzheimer's disease, inflammation, or excessive myelin uptake with PU.1 inhibitors are disclosed.

### CROSS REFERENCE TO RELATED APPLICATIONS

This application claims the benefit under 35 U.S.C. § 119(e) of the filing date of U.S, Patent Application Ser. No. 62/900,328, filed Sep. 13, 2019. The contents of the above-referenced application is hereby incorporated herein in its entirety by reference.

### BACKGROUND OF THE INVENTION

PU.1 protein (also referred to herein simply as PU.1) is a protein in humans encoded by the SPI1 gene. PU.1 is an ETS-domain transcription factor (e.g., activator) that activates gene expression implicated during myeloid, macrophage, and B-lymphoid cell development, as well as pre-mRNA splicing. It binds purine-rich sequences known as PU-boxes found on enhancers of target genes to regulate expression. PU.1 also has specificity determined by regions with high A/T concentrations. PU.1 is a key regulator of microglial cells and neuroinflammatory processes during neurodegeneration, however treatments directed to PU.1, or disorders related thereto, are lacking.

### SUMMARY OF THE INVENTION

In the present disclosure, high throughput screening methods to identify inhibitors which modulate PU.1 are disclosed, as well as, PU.1 inhibitors and methods of treatment of disorders related to PU.1 expression and activity.

The details of one or more embodiments of the invention are set forth in the description below. Other features or advantages of the present invention will be apparent from the following drawings and detailed description of several embodiments, and also from the appended claims.

In one aspect, the disclosure relates to a recombinant vector comprising a reporter gene under the control of at least two binding motifs capable of binding PU.1 protein. In some embodiments, the reporter gene is capable of expressing luciferase.

In some embodiments, the at least two binding motifs comprise lambda-beta binding motifs (LBB motifs). In some embodiments, the at least two binding motifs comprise three LBB motifs. In some embodiments, the at least two binding motifs comprise four LBB motifs. In some embodiments, the at least two binding motifs comprise five LBB motifs. In some embodiments, the at least two binding motifs comprise more than five LBB motifs.

In some embodiments, the at least two binding motifs are arranged in tandem. In some embodiments, the LBB motifs are arranged in tandem. In some embodiments, the at least two binding motifs are within a promoter region.

In some embodiments, the at least two binding motifs are Adenine (A)/Thymine (T) rich. In some embodiments, A or T comprise greater than 60% of the nucleotides of the at least two binding motifs.

In some embodiments, the at least one of the at least two binding motifs have at least 80% sequence identity to SEQ ID NO: 1. In some embodiments, the at least one of the at least two binding motifs have at least 90% sequence identity to SEQ ID NO: 1. In some embodiments, the at least one of the at least two binding motifs have at least 95% sequence identity to SEQ ID NO: 1. In some embodiments, the at least one of the at least two binding motifs comprises SEQ ID NO: 1.

In some embodiments, the at least two binding motifs have at least 80% sequence identity to SEQ ID NO: 2 or SEQ ID NO: 6. In some embodiments, the at least two binding motifs have at least 90% sequence identity to SEQ ID NO: 2 or SEQ ID NO: 6. In some embodiments, the at least two binding motifs comprise, consist essentially of or consist of SEQ ID NO: 2: or SEQ ID NO: 6.

In some embodiments, the at least two binding motifs comprise SEQ ID NO: 3, wherein X₁ is a nucleic acid sequence of ₅₋₁₅ nucleotides, wherein at least 50% of the nucleotides of X₁ are A or T, and wherein X₂ is a nucleic acid sequence of at least 5 nucleotides, wherein at least 50% of the nucleotides of X₂ are A or T. In some embodiments, at least 8 nucleotides of X₁ are A or T. In some embodiments, at least 65% of the nucleotides of X₁ are A or T. In some embodiments, X₁ comprises SEQ ID NO: 4. In some embodiments, at least 6 nucleotides of X₂ are A or T. In some embodiments, at least 65% of the nucleotides of X₂ are A or T. In some embodiments, X₂ comprises SEQ ID NO: 5.

In some embodiments, the recombinant vector is a pGL4.23 luciferase plasmid. In some embodiments, the recombinant vector is a ROSA26-1 plasmid (pROSA26-1). In some embodiments, the recombinant vector further comprises a Neomycin/G418 resistance gene under the control of a promoter.

In another aspect, the disclosure relates to an isolated cell comprising the recombinant vector as disclosed herein. In some embodiments, the isolated cell is selected from the group comprising: cells from the hematopoietic lineages including both primary from any organism or derived from stem cells, such as a microglial cell, monocytic cell, macrophage cell, T-cells, B-cells, NK-cells, eosinophil cell, neutrophil cell, hematopoietic stem cell, granulocyte cell, dendritic cell, innate lymphoid cell, megakaryocyte cell, myeloid derived suppressor cell, astrocytes, oligodendrocytes, oligodendrocytes precursor cells, and any immortalized lines derived therefrom, including BV2, N9, THP-1, Jurkat cell, Kasumi-1, leukemia cell and their derivatives. In some embodiments, the isolated cell is a microglial BV2 cell.

In another aspect, the disclosure relates to a method of screening compounds to identify a PU.1 protein inhibitor comprising: (a) exposing an isolated cell comprising the recombinant vectors described herein to a compound; (b) incubating the exposed isolated cell of step (a); and (c) quantifying the level of reporter activity of the isolated cell relative to a predetermined level of reporter activity, wherein, a lower level of reporter activity, relative to the predetermined level indicates that the compound is a PU.1 protein inhibitor.

In some embodiments, the method further comprises, excluding false positive results by additional steps (d) and (e), comprising: (d) exposing control cells which constitutively produce reporter to the compound; and (e) quantifying a level of reporter activity of the exposed control cells, wherein, a change of reporter activity of the exposed isolated cell equal to or less than a change of reporter activity of the exposed control cells indicates a false positive.

In some embodiments, the reporter gene is a gene capable of expressing luciferase and wherein the isolated cell which constitutively produce reporter are HEK-293 cytomegalovirus (CMV)-Luciferase cells.

In some embodiments, the method further comprises, quantifying the level of reporter messenger RNA (mRNA) of the exposed isolated cell.

In another aspect, the disclosure relates to a method of screening compounds to identify a PU.1 inhibitor comprising: (a) contacting a cell with a compound, wherein the cell includes a reporter construct having a reporter gene and a promoter region, wherein the promoter region comprises a PU.1 binding site and quantifying the level of reporter activity of the cell relative to a predetermined level of reporter activity; (b) performing an assay to determine whether the reporter activity detected in the cell is a false positive; (c) if the activity is not a false positive, calculating an effective area under the curve as an indicator of strong activity, wherein the effective area under the curve is calculated by determining a value of percent cell death and a value of percent reduced reporter activity and subtracting the value of percent cell death from the value of percent reduced reporter activity; and (d) determining whether the compound significantly reduces PU.1 dependent gene expression, wherein, a compound that is not a false positive, has an effective area under the curve, and reduces PU.1 dependent gene expression, is a PU.1 inhibitor.

In some embodiments, method of screening further comprises, performing the method on a plurality of compounds to identify a plurality of PU.1 inhibitors and identifying a structure activity relationship to identify common functional groups in the plurality of PU.1 inhibitors.

In some embodiments, the disclosure relates to a library of compounds, wherein each compound in the library comprises a common functional group identified according to the method of screening a plurality of compounds.

In another aspect, the disclosure relates to pharmaceutical compositions comprising:

a compound of any one of Formulae Ito IV, or a pharmaceutically acceptable salt, solvate, hydrate, polymorph, co-crystal, tautomer, stereoisomer, isotopically labeled derivative, or prodrug thereof; and

one or more pharmaceutically acceptable excipients.

In another aspect, the disclosure relates to additional pharmaceutical compositions as described herein.

In another aspect, the disclosure relates to a method of treating a disorder in a subject, comprising: (a) identifying a subject who has, is at risk of having, or is suspected of having a disorder related to PU.1 expression; and (b) administering an effective amount of at least one compound identified as an inhibitor of PU.1 by the methods described herein.

In another aspect, the disclosure relates to a method of treating a disorder in a subject, comprising: (a) identifying a subject who has, is at risk of having, or is suspected of having a disorder related to PU.1 expression; and (b) administering an effective amount of at least one pharmaceutical composition described herein.

In some embodiments, the at least one compound identified as an inhibitor of PU.1 administered is selected from Table 1. In some embodiments, the at least one compound identified as an inhibitor of PU.1 administered is A11: In

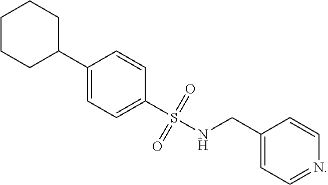 [SMILES: O=S(=O)(NCc1ccncc1)c1ccc(C2CCCCC2)cc1]

In some embodiments, the at least one compound identified as an inhibitor of PU.1 administered is of Formula V. In some embodiments, the at least one compound identified as an inhibitor of PU.1 administered is an additional compound as described herein. and pharmaceutically acceptable salts, hydrates, solvates, polymorphs, co-crystals, tautomers, stereoisomers, isotopically labeled derivatives, and prodrugs thereof.

In some embodiments, the method further comprises, (c) administering at least one additional compound, prior to, contemporaneously with, or subsequent to, the administration of the at least one compound identified as an inhibitor of PU.1. In some embodiments, the at least one additional compound is selected from the group comprising: an amyloid antibody or cognitive enhancer. In some embodiments, the at least one additional compound is an amyloid antibody. In some embodiments, the at least one additional compound is a cognitive enhancer.

In some embodiments, the disorder is selected from the group comprising: Alzheimer's disease (AD), inflammation, or excessive myelin uptake. In some embodiments, the disorder is AD.

A method for manufacturing a medicament of a compound of the invention for treating a disorder selected from the group comprising: AD, inflammation, or excessive myelin uptake is also provided.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure. In the drawings:

FIGS. 1 shows the design of the PU.1 reporter cell line.

FIGS. 2A-2F show the stable BV2 PU.1-Luciferase line and an overview of the selection process. FIG. 2A shows a PU.1 binding motif, the LBB motif into the pGL4.23 luciferase plasmid. FIG. 2B shows the composition of a reporter cell line. Five tandem copies of the PU.1-binding motif (5XλB) were inserted into the luciferase promoter of the pGL4.23 plasmid, followed by subcloning into the pROSA26-1 plasmid for stable integration into chromosome 6 of the immortalized mouse microglial BV2 cell line upon G418 selection. FIG. 2C: Left panel: BV2 cells with the λB motif (column 2 as read from left to right, n=6) have increased luminescence signal compared to identical BV2 cells lacking the motif (column 1 as read from left to right, n=9). Student's unpaired, two-sided t-test, ****P≤0.0001. Middle panel: Without the λB motif, knocking down (shRNA-1, n=3 and shRNA-2, n=3) or increasing (pcDNA PU.1, n=6) PU.1 expression did not affect luminescence levels versus control (n=9). Right panel: Knocking down PU.1 (shRNA-1, n=3 and shRNA-2, n=3) reduced luminescence in the BV2 PU.1-Luciferase line (control, n=12) and overexpressing PU.1 (pcDNA PU.1, n=6) increases luminescence. ANOVA followed by Dunnett's post hoc test versus control, ****P≤0.0001. FIG. 2D shows the compound solvent DMSO (1:1000, n=80 for both lines) had no effect on luminescence (left column of each set of two columns respectively) or cell viability (right column of each set of two columns respectively, Hoechst, 1:1000) versus untreated cells (Mock, n=60 for both lines) in the BV2 PU.1-Luciferase cells or the commercially available HEK293 CMVLuciferase cell line that expresses luciferase ubiquitously. Actinomycin D (Act. D, 1 μM, n=35 for both lines) was toxic to both cell lines, whereas puromycin (1 μM, n=24 (BV2) and 16 (HEK)) was ineffective in the resistant HEK293 CMV-Luciferase line. The known luciferase quencher 119113 (1 μM, n=5 for both lines) reduced luciferase in both lines, without affecting cell viability. The PU.1 inhibitor DB2313 (1μM, n=127 (BV2) and 61 (HEK)) reduced luminescence and cell viability only in the BV2 PU.1-Luciferase line. Student's unpaired, two-sided t-test, ****P≤0.0001. FIG. 2E shows an illustration of the highthroughput drug screen . FIG. 2F shows the Z-score of the 58100 tested compounds for luminescence (left panel) and cell viability (right panel). A11 is indicated in both panels. n=196 for DMSO and Act. D in both panels.

FIGS. 3A-3D show a summary of drug library screening and validation after round 1 and 2 of the 3-step validation. Round 1 eliminates non-specific luciferase quenchers by use of a commercially available HEK293 CMV-Luciferase cell line that expresses luciferase constitutively—any drug quenching luminescence in this line is eliminated from further testing. Round 2 eliminates statistically ineffective compounds by integrating the percent luminescence reduced from 600 pM to 2.5 μM in half-step dilutions and subtracting the cell viability reduced over the same concentration range, yielding a so-called effective area under the curve (AUC). FIG. 3A shows a graphical representation of the calculation of the AUC. Calculation of effective area under the curve (AUC) was performed by subtracting Hoechst signal AUC from luminescence signal AUC between 600 pM to 2.5 μM in twelve 1:2 dilution steps. FIG. 3B shows the numerical effects of the two rounds of screening and validation. FIG. 3C: Left panel: Elimination of luciferase quenchers based on P value of the Effective AUC in HEK293 CMV-Luciferase cells (n≤4 for all compounds) versus DMSO by use of ANOVA followed by Dunnett's post hoc test. Right panel: Selection of effective compounds based on P value of Effective AUC in BV2 PU.1-Luciferase cells (n≤8 for all compounds) versus DMSO by use of ANOVA followed by Dunnett's post hoc test. FIG. 3D shows a table describing the remaining 40 compounds (column 1), their separation into 6 groups (column 2) based on their effect on mRNA levels as measured by RTqPCR after a 2-day treatment in BV2 PU.1-Luciferase cells (n≤3 for all conditions, columns 3-8), origin (column 9), IC50 of the concentration-response curve in FIG. 3A (column 10), maximal effect Emax (column 11), effective AUC (column 12), grouping according to commonalities in their molecular structure (column 13), and molecular identifier on PubChem (column 14). Only compounds that significantly reduced luciferase mRNA levels were included.

FIGS. 4A-4B show the results after round 2 of the 3-step validation. FIG. 4A shows the resulting hits from each library screened. FIG. 4B shows the effective area under the curve (AUC) for the top 30 compounds from two libraries shown.

FIG. 5 shows a summary and description of libraries and hit rates from primary screen, followed by 3 steps of validation resulting in a final hit rate of 0.07%. Molecules in the Selleck library have properties generally known to have known mechanisms and FDA approval. Molecules in the Asinex1 and ChemDiv6 libraries generally have unknown properties, but are generally highly diversi in structures.

FIG. 6 shows an IC50 profile of the top hits from the Asinexl library from 600 pM to 10 μM. for all compounds.

FIG. 7 shows data that was fitted with Hill's equation. This information was used to obtain structure-activity-relationships for the validated 84 compounds.

FIG. 8 shows an example of the resulting molecular clusters on the left derived from the respective compounds in the center and on the right.

FIGS. 9A-9H show the evaluation of All, an inhibitor of PU.1 activity (identified herein) with nanomolar potency. FIG. 9A shows six of the most potent compounds (and the PU.1 inhibitor DB2313 in column 1), grouping (column 2) and mRNA fold change over DPBS treated cells (n≤3 for all conditions, columns 3-8), source compound library (column 9), IC50 (the concentration at with 50% of the maximal inhibitory effect occurs) based on luminescence inhibition in BV2 PU.1-Luciferase cells (column 10), and IC50 based on inhibition of Zymosan A (column 11) and mouse myelin (column 12) uptake in iPS-microglia. FIG. 9B shows the calculation of IC50 was performed using Hill's equation with baseline fixed to 0. FIG. 9C shows compound structures. FIG. 9D shows a concentration response curve for the same compounds in human induced pluripotent stem cell derived microglia (iPS-microglia) for uptake of pHrodo-labelled Zymosan A bioparticles (left panel) and mouse myelin (right panel). FIG. 9E shows a table of IC50 values for all compounds in the uptake assay in iPS-microglia. IC50 was calculated using Hill's equation with baseline fixed to 0. FIG. 9F shows a concentration response curve in iPS-microglia for determining the cytotoxic dose of A11 by use of Caspase 3/7 activity (luminescence) and percent propidium iodide positive cells. IC50 of cell viability determined using Hill's equation with baseline fixed to 0. FIG. 9G shows propidium iodide staining in vehicle (top left), A11 at 20 nM (top right), A11 at 150 nM (bottom left) and A11 at 250 nM (bottom right). Staining identified with arrows indicate propidium iodide. Scale bar 20 μm. FIG. 9H shows a concentration response curve in iPS-microglia for PU.1-dependent gene expression by use of RTqPCR.

FIGS. 10A-10D show anti-inflammatory properties of A11 after IFNγ treatment on human induced pluripotent stem cell derived microglia. Cell viability is shown in FIG. 10A as measured with CellTiter Glo (Promega) in untreated (vehicle), A11 (2.5 μM) treated, IFNγ (25 ng/mL) treated and A11 +IFNγ treated cells. The morphology (round represent un-activated and elongated, filamentous represent activated by IFNγ) of these same conditions is shown in in FIGS. 10B and quantified in FIG. 10C. Gene expression changes of the same conditions are shown in FIG. 10D, by use of RTqPCR.

FIGS. 11A-11B show percent luminescence achieved in luciferase-quenching control experiments using BV2 PU.1-Lucifersase lysate (FIG. 11A) and HEK293 CMV-Luciferase lysate (FIG. 11B). Lysate from BV2 PU.1-Luciferase (FIG. 11A) and HEK 293 CMV-Luciferase (FIG. 11B) cells did not lose luminescence signal upon a 10 min exposure to either of the remaining compounds, except for 119113 (n≤4 for all conditions and both cell lines). ANOVA followed by Dunnett's post hoc test versus DMSO, ****P≤0.0001.

FIGS. 12A-12B show the effects of A11 on cell viability and myelin uptake after 48 h. FIG. 12A shows a concentration response curve for cell viability and myelin uptake in iPSC-microglia. FIG. 12B shows mylein uptake measured by uptake of pHrodo-GFP-labelled wild-type mouse myelin particles.

FIG. 13 shows criteria for positive hit in PU.1 screen. Overall positive score means column AN=“Y”. Overall negative score means column AN=“”. The variables in FIG. 13 refer to columns in a calculation performed in Microsoft Excel™. A=Plate; B=Well; C=Type; E=CrosstalkCorrected-Luminesence_A; F=Luminescence_A; G=CrosstalkCorrected-Luminesence_B; H=Luminescence_B; I=TotalNumberObjects_A; J=TotalFluorescenceArea_A; K=TotalNumberObjects_B; L=TotalFluorescenceArea_B; M=Xwell_RepA; N=Xwell_RepB; O=Xwell FluorArea RepA; P=Xwell FluorArea RepB; V=Xwell-Median-RepA; W=Xwell-Median-RepB; X=Xwell-Median-FA-A; Y=Xwell-Median-FA-B; Z=Zscore Rep A; AA=Zscore Rep B; AB=Zscore FA RepA; AC=Zscore FA RepB; AD=MAD-Zscore RepA; AE=MAD-Zscore RepB; AF=MAD-Zscore FA-RepA; AG=MAD-Zscore FA-RepB; AH=Hit Zscore RepA; AI=Hit Zscore RepB; AJ=Hit Zscore FA RepA; AK=Hit Zscore FA RepB; AL=Zscore Lumin Positive; AM=Zscore FA Positive; and AN=Overall Screen Positive.

FIGS. 14A-14I shows that compound A11 obtained from several vendors is efficacious in multiple cell lines starting at 3 hours after application. FIG. 154: A11 obtained from Asinex, Mcule, VitasM and AKoS shows similar RTqPCR gene modulation profile in BV2 PU.1-Luciferase cells. The gene modulation was reproduced in unmodified BV2 cells and primary mouse microglia. FIG. 14B: Concentration-response curve of A11 from multiple vendors, measured as percent reduction of luminescence. Non-linear fit with Hill's equation with baseline fixed to 0. FIG. 14C: Time course for reduction of mRNA levels of luciferase and il1β as well as luminescence emission in BV2 PU.1-Luciferase cells. FIGS. 14D-14E: Time and concentration-response curve (of pHrodo-labelled particles) for uptake of pHrodo-labelled Zymosan A (FIG. 14D) and mouse myelin in (FIG. 14E) in BV2 cells (left panels, respectively in each FIGS. 14D-14E), and iPS-microglia (right panels, respectively in each FIGS. 14D-14E). FIGS.: 14F-14G: Time and concentration-response curves of A11 (FIG. 14F) and DB2313 (FIG. 14G) of Zymosan A uptake in BV2 cells. Left, middle, and right panels show 1, 2 and 3 hours after compound application, respectively. ANOVA followed by Dunnett's post hoc test versus vehicle, black bars indicate P≤0.05. FIG. 14H shows flow cytometry analysis of microglia derived from induced pluripotent stem cells (left panel), quadruple immunocytochemistry for Hoechst and IBA1 (middle panel), and TMEM119 and PU.1 (right panel). FIG. 14I shows flow cytometry analysis of microglia derived from embryonic stem cells (left panel), Quadruple immunocytochemistry for Hoechst and IBA1 (middle panel), and TMEM119 and PU.1 (right panel).

FIG. 15 shows the identification of the target of A11 with gene expression analysis and cellular thermal shift assay. A heat map is shown representing RTqPCR results on PU.1-related genes in iPS-microglia (n≤3 for all treatments) after a 2-day treatment with DPBS (10 μL/mL), IL4 (100 ng/mL, box indicates ES-microglia), CX₃CL1 (100 ng/mL), CD200 (100 ng/mL), IL13 (100 ng/mL), TGFβ (25 ng/mL), IL12 (5 ng/mL), IL1β (25 ng/mL), PMA (10 ng/mL), IL33 (10 ng/mL), IFNβ (10 ng/mL), IFNα (25 ng/mL), LPS (25 ng/mL), Activin A (50 ng/mL), IL3 (50 ng/mL), CCL5 (100 ng/mL), ATP (100 nM), TNFα (25 ng/mL), IFNγ (25 ng/mL), IL10 (100 ng/mL), and IL6 (50 ng/mL). Left column indicates application of cytokine or molecule alone, and right column indicates additional application of A11 (20 nM). Cycle number was normalized to β-ACTIN. Student's unpaired, two-sided t-test, open black circles represent P≤0.05 after a treatment with inflammatory molecules alone versus DPBS alone, and filled black circles represent P≤0.05 after co-application of the inflammatory molecule (or DPBS) and A11 (20 nM) versus the inflammatory molecule (or DPBS) alone.

FIGS. 16A-16E show that A11 reverses the effects of inflammatory molecules in iPS and ES-microglia. FIG. 16A shows epifluorescence micrographs of the cellular membrane of iPS-microglia after labelling with Vybrant DiI (red channel). Top row: Effect of a 2-day treatment with DPBS (10 μL/mL), IFNγ (25 ng/mL), TNFα (25 ng/mL), TGFβ (25 ng/mL), IL1β (25 ng/mL), LPS (25 ng/mL), ATP (100 nM), CD200 (100 ng/mL), IFNα (25 ng/mL), IL12 (5 ng/mL), CX₃CL1 (100 ng/mL), CCL5 (100 ng/mL), PMA (10 ng/mL), IFNβ (10 ng/mL), IL33 (10 ng/mL), and IL4 (100 ng/mL). n=52 for DPBS, n=9 for all other inflammatory molecules, except IL33 where n=3. Bottom row: effect after co-application with A11 (20 nM). Scale bar 10 μm. FIG. 16B shows quantification of the surface area increase by dividing the total Vybrant DiI-positive surface area by number of Hoechst-positive cells per field of view after treatment (left columns of each set of two), and the effect of A11 (right column of each set of two) for iPS (left panel) and ES-microglia (right panel). Student's unpaired, two sided t-test, ****P≤0.0001, ***P≤0.001, **P≤0.01, *P≤0.05. FIG. 16C shows epifluorescence micrographs of intracellular neutral lipid accumulation by staining with BODIPY (green channel) in iPS-microglia. Top row: An increase in number of cells per well with ≥2 BODIPY-positive structures was observed after a 2-day treatment with IFNγ (25 ng/mL), TNFα (25 ng/mL) and IL33 (10 ng/mL) as compared to DPBS treatment. Bottom row: A11 treatment reversed this increase. FIG. 16D shows a quantification of the number of cells with ≥2 BODIPY-positive structures divided by number of Hoechst-positive cells per field of view after treatment (left columns of each set of two), and the effect of A11 (right columns of each set of two) for iPS (left panel) and ES-microglia (right panel). Student's unpaired, two-sided t-test, ***P≤0.001, **P≤0.01, *P≤0.05. FIG. 16E shows ATP levels as measured by adding CellTiter-Glo to cells in media and measuring luminescence per well. A 2-day treatment with IFNγ (25 ng/mL) caused increase in ATP levels that was reversed by A11 (20 nM) treatment in iPS (left panel) and ES-microglia (right panel). Student's unpaired, twosided t-test, **P≤0.01. FIG. 16F shows the results of an ELISA for IL1β was performed on 50 μL supernatant samples obtained from ≥500 000 cells in 500 μL media. A 2-day treatment with IFNγ (25 ng/mL) increased supernatant IL1β levels, which was reversed by A11 (20 nM) treatment in iPS (left panel) and ES-microglia (right panel). Student's unpaired, two-sided t-test, **P≤0.01, *P≤0.05.

FIGS. 17A-17H show results from comparing the effects of multiple inflammatory molecules in iPS-microglia. FIG. 17A shows the average cellular membrane surface area of iPS-microglia was increased after a 2-day treatment with IFNγ (25 ng/mL), TNFα (25 ng/mL), LPS (25 ng/mL) and IL1β (25 ng/mL) as compared to DPBS treatment. Quantification based on total Vybrant DiI-positive surface area divided by number of Hoechst positive cells per field of view. ANOVA followed by Dunnett's post hoc test versus DPBS (column labeled DPBS, 12^{th} column from the left), first four columns from left indicate P≤0.05. FIG. 17B shows epifluorescence micrographs of the cellular membrane of iPS-microglia after labelling with Vybrant DiI (red channel) and Hoechst (blue channel) after treatment with inflammatory molecules. Scale bar is 50 μm. FIG. 17C shows no effects of A11 (≤20 nM) on the average cell size versus DPBS treatment in iPS (left panel) and ES-microglia (right panel). ANOVA followed by Dunnett's post hoc test versus DPBS, ****P≤0.0001. FIG. 17D shows the average number of cells containing ≥2 BODIPY-positive structures per well of 5000 cells was increased by a 2-day treatment with IFNγ (25 ng/mL), TNFα (25 ng/mL) and IL33 (10 ng/mL) as compared to DPBS treatment. ANOVA followed by Dunnett's post hoc test versus DPBS (last column from left), first three columns from left indicate P≤0.05. FIG. 17E shows epifluorescence micrographs of BODIPY-positive structures (green channel) and Hoechst staining (blue channel). FIG. 17F shows no effect of A11 (<20 nM) on number of cells containing ≥2 BODIPY-positive structures in iPS (left panel) and ES-microglia (right panel). ANOVA followed by Dunnett's post hoc test versus DPBS. FIG. 17G shows an increase in ATP levels per well was observed after a 2-day treatment with IFNγ (25 ng/mL) as measured by luminescence using the CellTiter-Glo reagent. ANOVA followed by Dunnett's post hoc test versus DPBS (labled DPBS, 8^{th} column from left), the first column indicates P≤0.05. FIG. 17H shows no effect of A11 (≤20 nM) on amount of ATP per well in iPS (left panel) and ES-microglia (right panel). ANOVA followed by Dunnett's post hoc test versus DPBS, ****P≤0.0001. FIG. 17I shows a 2-day treatment A11 (20nM) did not affect IL1β concentration in the supernatant as measured by ELISA performed on 50 μL samples of supernatants from 500 μL media of ≥500 000 cells. Student's unpaired, two-sided t-test.

FIGS. 18A-18Z show chemical strutures of various compounds, the Compound CIDs are Pubmed Compound CID numbers and correlate with the “identifier” number found in the rightmost column of FIG. 3D. FIG. 18A shows 1A2 Compound ID (CID) 1183879. FIG. 18B shows 1A5 CID 1183806. FIG. 18C shows 1B5 CID 2911006. FIG. 18D shows 1C4 CID 7770-4005. FIG. 18E shows 1D3 CID 15987842. FIG. 18F shows 1G9 CID 16008936. FIG. 18G shows 1H7 CID 16009506. FIG. 18H shows 2A7 CID 16012528. FIG. 18I shows 2A9 CID 16012592. FIG. 18J shows 2C2 CID 16020759. FIG. 18K shows 2C3 CID 16021553. FIG. 18L shows 2C12 CID E955-1348. FIG. 18M shows 2D3 CID 16024095. FIG. 18N shows 2D5 CID 16024464. FIG. 18O shows 2E4 CID 16029297. FIG. 18P shows 2E6 CID 16030548. FIG. 18Q shows 2E7 CID 16030594. FIG. 18R shows 2E10 CID 16030655. FIG. 18S shows 2F₃ CID 16031649. FIG. 18T shows 2F₅ CID 16031733. FIG. 18U shows 2F₇ CID 16031805. FIG. 18V shows 2F₉ CID 16032778. FIG. 18W shows 2F₁₁ CID 7598405. FIG. 18X shows 2G2 CID 16033259. FIG. 18Y shows 2G8 CID 3309351. FIG. 18Z shows 2G10 CID 16034073.

FIGS. 19A-19S show chemical structures of various compounds, the Compound CIDs are Pubmed Compound CID numbers and correlate with the “identifier” number found in the rightmost column of FIG. 3D. FIG. 19A shows 2G12 CID 4174345. FIG. 19B shows 2H2 CID 4174346. FIG. 19C shows 2H3 CID 4175695. FIG. 19D shows 2H5 CID 5301786. FIG. 19E shows A09 CID 1566703. FIG. 19F shows A11 CID 6459432. FIG. 19G shows B03 CID 6490146. FIG. 19H shows Betamethasone (CID 9782). FIG. 19I shows D04 CID 1176530. FIG. 19J shows D06 CID 652457. FIG. 19K shows D09 CID 6460157. FIG. 19L shows D10 CID 1448468. FIG. 19M shows Dexamethasone (CID 5743). FIG. 19N shows E01 CID 2864692. FIG. 19O shows Loteprednol (CID 129010398). FIG. 19P shows Mometasone (CID 129010385). FIG. 19Q shows Mubritinib (CID 6444692). FIG. 19R shows Trametinib (CID 11707110). FIG. 19S shows Wortmannin (CID 312145).

### DETAILED DESCRIPTION OF THE INVENTION

The term “binding motif,” as used herein, refers to a nucleotide sequence (e.g., the lambda-beta binding motif of SEQ ID NO: 1; or multimers thereof, i.e., SEQ ID NO: 2, 3 or 6) which has a specific biological significance (e.g., binds to a specific molecule to affect a specific effect). For example, the lambda-beta binding motif belongs to a group of motifs that specifically recognize and bind to transcription factors, referred to as PU.1. In some embodiments a lambda-beta binding motif has a middle 5′-GGAA-3′ sequence with flanking A/T rich sequences crucial for conferring PU.1 selective binding. The flanking sequences bind unique structural properties of PU.1 and can therefore be targeted by drugs acting as allosteric inhibitors that prevent PU.1 from binding to the a motif. In some embodiments, the A/T rich sequence is a sequence targeted by PU.1. In some embodiments, the A/T rich sequence is a sequence targeted DB2313.

The term “gene,” as used herein, refers to a sequence of nucleotides in a nucleic acid (e.g., DNA, RNA) that codes for a molecule (e.g., protein) with a function.

The term “isolated cell,” as used herein, refers to a cell that can host, replicate, and express a vector described herein (e.g., a vector comprising a nucleic acid molecule including a motif capable of binding PU.1). As used herein, the term “isolated,” refers to the characteristic of a material as provided herein being removed from its original or native environment (e.g., the natural environment if it is naturally occurring). Therefore, a naturally-occurring cell present in a living animal is not isolated, but the same cell, separated by human intervention from some or all of the coexisting materials in the natural system, is isolated. An artificial or engineered cell, for example, may be a non-naturally occurring cell comprising a heterologous nucleic acid construct, such as the expression constructs and vectors described herein, are, accordingly, also referred to as isolated. A cell does not have to be purified in order to be isolated. Accordingly, a cell may be part of a composition, and still be isolated in that such composition is not part of the environment in which the material is found in nature.

The term “plasmid,” as used herein, are well known in the art, but are generally known to be small DNA molecules which are independent and separate from the chromosomal DNA, and are also self-replicating. Plasmids are most commonly found to be small circular double stranded DNA molecules, which typically do not carry any essential nucleic acid sequences (e.g., genes), but rather carry auxiliary or acquired genes such as nucleic acids encoding antibiotic resistance genes as well as transgenes incorporated artificially. Plasmids (both natural and artificial) are very useful and common delivery mechanisms (e.g., vectors) in molecular cloning for their ability to introduce foreign nucleic acids into a host cell. At their most basic, a plasmid vector will comprise an origin of replication, promoter region, and insert (e.g., transgene). Examples of plasmids are the pGL4.23 luciferase plasmid (commercially available, e.g., Promega™) and ROSA26-1 plasmid (commercially available, e.g., Addgene™).

The term “promoter” is art-recognized and refers to a nucleic acid molecule with a sequence recognized by the cellular transcription machinery and able to initiate transcription of a downstream gene. A promoter can be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect.

The term “reporter gene” as used herein, (also may be referred to herein, as a “reporter”) is a gene under the control of a regulatory sequence (e.g., promoter, binding motifs) of another gene of interest which because the characteristics of their expression (or repression), organisms expressing them (or failing to express them) are easily identified, measured, or selectable. Being under the control of another gene of interest confers identification of the reporter, as identification of the gene of interest. For example, a reporter gene can be used to indicate if a certain gene has been taken up by or expressed in the cell or organism population. Examples are luciferase reporters, which use luminescence to mark (i.e., identify, allow measurement or quantification) expression.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “vector,” as used herein, refers to a nucleic acid that can be modified to deliver a nucleic acid sequence of interest that is able to enter into a cell, mutate and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

As used herein the term “wild-type” is a term of the art understood by skilled persons and means the typical form of an organism, strain, gene or characteristic as it occurs in nature as distinguished from mutant or variant forms.

PU.1 protein (also referred to herein simply as PU.1) is a protein in humans encoded by the SPI1 gene. PU.1 is an ETS-domain transcription factor (e.g., activator) that activates gene expression implicated during myeloid, macrophage, and B-lymphoid cell development, as well as pre-mRNA splicing. It binds purine-rich sequences known as PU-boxes found on enhancers of target genes to regulate expression. PU.1 also has specificity determined by regions with high Adenine (A)/Thymine (T) concentrations. PU.1 is a key regulator of microglial cells and neuroinflammatory processes during neurodegeneration, however treatments directed to PU.1, or disorders related thereto, are lacking. In the present disclosure, high throughput screening methods to identify inhibitors which modulate PU.1 are disclosed, as well as, PU.1 inhibitors and methods of treatment of disorders related to PU.1 expression and activity.

Accordingly, in one aspect, the disclosure relates to a recombinant vector comprising a reporter gene under the control of at least two binding motifs capable of binding PU.1. By placing the reporter under the control of the binding motifs, activity of the reporter indicates the presence of PU.1. The reporter gene can be placed anywhere under the control of the at least two binding motifs. Multiple reporter genes may also be used to confer additional capabilities. Further, other genes and other components may be under the control of the same at least two binding motifs. In some embodiments, the recombinant vector comprises at least one reporter gene under the control of the at least two binding motifs. In some embodiments, the recombinant vector comprises at least two reporter genes under the control of the at least two binding motifs. In some embodiments, the recombinant vector comprises multiple copies of the same reporter gene under the control of the at least two binding motifs. In some embodiments, the recombinant vector comprises at least two different reporter genes placed under the control of the at least two binding motifs. In some embodiments, the reporter gene is selected from the group comprising: lacZ, cat, gfp, rfp, and a gene capable of expressing luciferase. In some embodiments, the reporter gene is capable of expressing luciferase. In some embodiments, the recombinant vector comprises at least two reporter genes under the control of at least two biding motifs, wherein at least one of the reporter genes is capable of expressing luciferase. In some embodiments, the recombinant vector comprises 5 copies of a PU.1 bidning motif, wherein the binding motif is a lambda beta motif, which copies are positioned upstream of a reporter, wherein the reporter is luciferase based, which is integrated into the Rosa26 locus, wherein the PU.1 bindng site (e.g., motif) is flanked by A/T rich sequences which are targeted by DB2313.

In some embodiments, the at least two binding motifs comprise lambda-beta binding motifs (LBB motifs). In some embodiments, the at least two binding motifs comprise three LBB motifs. In some embodiments, the at least two binding motifs comprise four LBB motifs. In some embodiments, the at least two binding motifs comprise five LBB motifs. In some embodiments, the at least two binding motifs comprise more than five LBB motifs.

The at least two binding motifs may be arranged in any manner chosen by one of ordinary skill in the art, optionally in tandem. Arrangement in tandem, as used herein, refers to arrangement or repeat of the sequences or genes of interest (e.g., binding motifs, LBB motifs) in such a manner that they are adjacent to each other. In some embodiments, the at least two binding motifs are not arranged in tandem. In some embodiments, the at least two binding motifs are arranged in tandem. In some embodiments, the LBB motifs are not arranged in tandem. In some embodiments, the LBB motifs are arranged in tandem. In some embodiments, where there are more than two binding motifs, at least two of the binding motifs are arranged in tandem while others are not. In some embodiments, where there are more than two LBB motifs, at least two of the LBB motifs are arranged in tandem while others are not.

The binding motifs (e.g., LBB motifs) may reside anywhere to facilitate the desired effect of reporter under their control (e.g., luciferase gene), but commonly reside in the non-coding region (e.g., promoter) of the gene of interest. In some embodiments, the at least two binding motifs are within a promoter region.

In some embodiments, the binding motifs may be connected (e.g., linked by an interspersed sequence) by a spacer of one or more nucleotides. In some embodiments, the spacer comprises at least 1 and no more than 10 (e.g., 1, 2, 3, 4, 5, 6, 7, 8, 9, or 10) nucleotides. In some embodiments, the spacer is two nucleotides long. In some embodiments the spacer comprises a Guanine (G). In some embodiments, the spacer comprises a Cytosine (C). In some embodiments, the spacer comprises a GC. In some embodiments, there is a spacer between each of the at least two binding motifs. In some embodiments, when there is more than two binding motifs, there is a spacer between less than all of the binding motifs. In some embodiments, the binding motifs are LBB motifs and comprise spacers. In some embodiments, there are at least two binding motifs and spacers between each LBB motifs.

Flanking regions of the binding motifs can also impart PU.1 specificity. As described above, PU.1 has an affinity for A/T regions. Accordingly, in some embodiments, the at least two binding motifs are A/T rich. In some embodiments, A or T comprise at least 50% (e.g., at least 55%, at least 60%, at least 65%, at least 70%, at least 75%, at least 80%, at least 85%, at least 90%, at least 95%, at least 96%, at least 97%, at least 98%, at least 99%, at least 99.5%, at least 99.9%) of the nucleotides of the at least two binding motifs.

In some embodiments, at least one of the at least two binding motifs has at least 80% sequence identity (e.g., at least 80%, at least 85%, at least 90%, at least 95%, at least 96%, at least 97%, at least 98%, at least 99%, at least 99.5%, at least 99.9% sequence identity) to SEQ ID NO: 1. In some embodiments, at least one of the at least two binding motifs has at least 90% sequence identity to SEQ ID NO: 1. In some embodiments, at least one of the at least two binding motifs comprise SEQ ID NO: 1.

The terms “percent identity,” “sequence identity,” “% identity,” “% sequence identity,” and “% identical,” as they may be interchangeably used herein, refer to a quantitative measurement of the similarity between two sequences (e.g., nucleic acid or amino acid). The percent identity of genomic DNA sequence, intron and exon sequence, and amino acid sequence between humans and other species varies by species type, with chimpanzee having the highest percent identity with humans of all species in each category.

Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48: 1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol., 215, 403 (1990)).

When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%,at least 96%, at least 96.5%,at least 97%, at least 97.5%,at least 98%, at least 98.5%,at least 99%, at least 99.5%, at least 99.6%, at least 99.7%, at least 99.8%, at least 99.9% identity) and all increments thereof (e.g., tenths of a percent (i.e., 0.1%), hundredths of a percent (i.e., 0.01%), etc.).

In some embodiments, the at least two binding motifs has at least 80% sequence identity (e.g., at least 80%, at least 85%, at least 90%, at least 95%, at least 96%, at least 97%, at least 98%, at least 99%, at least 99.5%, at least 99.9% sequence identity) to SEQ ID NO: 2. In some embodiments, the at least two binding motifs has at least 90% sequence identity to SEQ ID NO: 2. In some embodiments, the at least two binding motifs comprise SEQ ID NO: 2.

In some embodiments, the at least five binding motifs has at least 80% sequence identity (e.g., at least 80%, at least 85%, at least 90%, at least 95%, at least 96%, at least 97%, at least 98%, at least 99%, at least 99.5%, at least 99.9% sequence identity) to SEQ ID NO: 6. In some embodiments, the at least five binding motifs has at least 90% sequence identity to SEQ ID NO: 6. In some embodiments, the at least five binding motifs comprise SEQ ID NO: 6.

In some embodiments, the at least two binding motifs comprise SEQ ID NO: 3, wherein X₁ is a nucleic acid sequence of ₅₋₁₅ nucleotides, wherein at least 50% (e.g., at least 55%, at least 60%, at least 65%, at least 70%, at least 75%, at least 80%, at least 85%, at least 90%, at least 95%, at least 96%, at least 97%, at least 98%, at least 99%, at least 99.5%, at least 99.9%) of the nucleotides of X₁ are A or T, and wherein X₂ is a nucleic acid sequence of at least 5 nucleotides, wherein at least 50% of the nucleotides of X₂ are A or T. In some embodiments, X₂ is at least 5 nucleotides, but less than or equal to 35 nucleotides. In some embodiments, X₂ is at least 5 nucleotides, but less than or equal to 25 nucleotides. In some embodiments, X₂ is at least 5 nucleotides, but less than or equal to 15 nucleotides. In some embodiments, at least 8 nucleotides of X₁ are A or T. In some embodiments, at least 65% of the nucleotides of X₁ are A or T. In some embodiments, X₁ comprises SEQ ID NO: 3. In some embodiments, at least 6 nucleotides of X₂ are A or T. In some embodiments, at least 65% of the nucleotides of X₂ are A or T. In some embodiments, X₂ comprises SEQ ID NO: 5.

In some embodiments, the recombinant vector is a pGL4.23 luciferase plasmid (available from Promega™; its sequence is accessible via PubMed: www.ncbi.nlm.nih.gov/nuccore/DQ904455). In some embodiments, the recombinant vector is a ROSA26-1 plasmid (pROSA26-1) (available via Addgene™; www.addgene.org/21714/sequences/). In some embodiments, the recombinant vector further comprises a Neomycin/G418 resistance gene under the control of a promoter.

In another aspect, the disclosure relates to an isolated cell comprising the recombinant vector as disclosed herein. Any suitable cell can be used for to practice the subject matter herein and can readily be selected by one of ordinary skill in the art for the intended application. Of particular interest are cells affected by PU.1 expression, which are implicated in myeloid, macrophage, and B-lymphoid cell development, as well as in microglial cells and neuroinflammatory processes during neurodegeneration. Microglial cells are macrophage cells and a type of glial cell located in the brain and spinal cord (e.g., neuroglial cells). They act as the primary active immune defense in the central nervous system (CNS) and, thus, play a large role in inflammation and immune regulation in the CNS. In some embodiments, the isolated cell is selected from the group comprising: cells from the hematopoietic lineages including both primary from any organism or derived from stem cells, such as a microglial cell, monocytic cell, macrophage cell, T-cells, B-cells, NK-cells, eosinophil cell, neutrophil cell, hematopoietic stem cell, granulocyte cell, dendritic cell, innate lymphoid cell, megakaryocyte cell, myeloid derived suppressor cell, astrocytes, oligodendrocytes, oligodendrocytes precursor cells, and any immortalized lines derived therefrom, including BV2, N9, THP-1, Jurkat cell, Kasumi-1, leukemia cell and their derivatives. In some embodiments, the isolated cell is a microglial BV2 cell.

Currently, there are no effective ways to efficiently screen compounds for PU.1 therapeutic inhibitory properties. Accordingly, in one aspect, the disclosure relates to a method of screening compounds to identify a PU.1 protein inhibitor comprising: (a) exposing an isolated cell comprising the recombinant vectors described herein to a compound; (b) incubating the exposed isolated cell of step (a); and (c) quantifying the level of reporter activity of the isolated cell relative to a predetermined level of reporter activity, wherein, a lower level of reporter activity, relative to the predetermined level indicates that the compound is a PU.1 protein inhibitor. By incorporating the reporter gene to operate under the influence of the binding motifs, if PU.1 is inhibited (i.e., cannot bind (e.g., due to allosteric inhibition), or is not available), it will not bind the binding motifs and fail to express the reporter gene product. The activity of the compound to inhibit PU.1 is marked and quantifiable by the reporter gene product. Thus, after exposing the isolated cell to a compound, it should be incubated for a period of time, to allow for inhibition of PU.1 to occur as well as processing of the PU.1 if uninhibited, sufficient to measure the effect on the reporter gene product. One of ordinary skill in the art can readily ascertain appropriate time frames for incubation, as well as use multiple time periods to establish inhibition times, without undue experimentation. In some embodiments the incubation period is between 12 hours and 96 hours. In some embodiments, the incubation period is between 36 and 60 hours. In some embodiments, the incubation period is 48 hours.

The incubated cells are then assessed for reporter gene activity, which is quantified and assessed relative to a predetermined value of reporter gene activity. Reporter genes are well known in the art, one of ordinary skill will readily understand the methods used to assess reporter gene activity in light of the selected reporter gene (e.g., by luminescence for luciferase). By using the method, one can assess the activity of the compound in inhibiting PU.1 through the quantification and comparison with the predetermined reporter gene activity. The predetermined level can be set as appropriate to indicate no PU.1 inhibition, for example, by quantifying the reporter gene activity in similar cell lines without the compound exposure or incubation (i.e., control cells). Accordingly, a lower level of reporter gene activity in the exposed cells than the predetermined level will indicate PU.1 inhibition. In some embodiments, the predetermined level of reporter gene activity is set by using control cells of similar configuration, not exposed to the compound.

It may further be of importance to ensure compounds screened are not otherwise inhibiting the reporter gene product (e.g., inactivation of the reporter gene product, quenching). This can be done by screening for false positives by quantifying the level of change in reporter activity of the exposed cells relative to the change in reporter activity of cells exposed to the same compound which constitutively produce (i.e., always produce) reporter gene product. In some embodiments, the method further comprises, excluding false positive results by additional steps (d) and (e), comprising: (d) exposing control cells which constitutively produce reporter to the compound; and (e) quantifying a level of reporter activity of the exposed control cells, wherein, a change of reporter activity of the exposed isolated cell equal to or less than a change of reporter activity of the exposed control cells indicates a false positive. In some embodiments, the reporter gene is a gene capable of expressing luciferase and wherein the isolated cell which constitutively produce reporter are HEK-293 cytomegalovirus (CMV)-Luciferase cells.

It may also be advantageous to quantify, either primarily by, or for confirmation of other quantifications, the reporter gene product by other means. One such way is by quantifying the levels of messenger RNA (mRNA) of the reporter gene in the exposed cells. There exist a multitude of ways to quantify the mRNA of the reporter gene in the exposed cells, which are all well known in the art (e.g., reverse transcriptase polymerase chain reaction (rtPCR)). In some embodiments, the method further comprises, quantifying the level of reporter messenger RNA (mRNA) of the exposed isolated cell. In some embodiments, the mRNA is measured by rtPCR.

Another method for screening compounds for PU.1 inhibitory properties, incorporates analysis to quantify the effectiveness of the compounds. This can be accomplished by measuring the effect of a compound (i.e., reduction in luminescence) over a range of concentrations (e.g., 600 pM to 2.5 microM) and correct for cellular death, yielding an effective area under the curve (AUC). The correction for cellular death is performed by subtracting the percent cellular death for a compound from its correlated percent effect on the reporter gene product (e.g., reduction in luminescence). The compounds can then be ranked according to effectiveness and qualified as: 1) weak; 2) medium; or 3) strong reporter gene product effectors. Weak effectors are between 2.5 and 3 (i.e., 2.5<y<3) standard deviations reduction from the mean value tested compounds; medium effectors are between 3 and 4 (i.e., 3<y<4) standard deviations reduction; and strong effectors are greater than 4 (i.e., >4) standard deviations difference form the mean; where the “y” is equal to the ((compound effect value) less the (mean value of compounds tested)) divided by the (standard deviation of the sample set) (i.e., y=(value−meanvalue)/stddev). Compounds are excluded if cell viability is reduced less than 2 (i.e., >2) standard deviations from the mean, using the same formula. This process can be iterated to find the most effective inhibitors.

Accordingly, in another aspect, the disclosure relates to a method of screening compounds to identify a PU.1 inhibitor comprising: (a) contacting a cell with a compound, wherein the cell includes a reporter construct having a reporter gene and a promoter region, wherein the promoter region comprises a PU.1 binding site and quantifying the level of reporter activity of the cell relative to a predetermined level of reporter activity; (b) performing an assay to determine whether the reporter activity detected in the cell is a false positive; (c) if the activity is not a false positive, calculating an effective area under the curve as an indicator of strong activity, wherein the effective area under the curve is calculated by determining a value of percent cell death and a value of percent reduced reporter activity and subtracting the value of percent cell death from the value of percent reduced reporter activity; and (d) determining whether the compound significantly reduces PU.1 dependent gene expression, wherein, a compound that is not a false positive, has an effective area under the curve, and reduces PU.1 dependent gene expression, is a PU.1 inhibitor. In some embodiments, the reporter gene is a gene capable of expressing luciferase.

In some embodiments, method of screening further comprises, performing the method on a plurality of compounds to identify a plurality of PU.1 inhibitors and identifying a structure activity relationship to identify common functional groups in the plurality of PU.1 inhibitors. In some embodiments, at least 2 compounds (e.g., at least 2; at least 3; at least 4; at least 5; at least 6; at least 7; at least 8; at least 9; at least 10; at least 11; at least 12; at least 13; at least 14; at least 15; at least 16; at least 17; at least 18; at least 19; at least 20; at least 25; at least 30; at least 35; at least 40; at least 45; at least 50; at least 60; at least 70; at least 80; at least 90; at least 100; at least 125; at least 150; at least 175; at least 200; at least 300; at least 400; at least 500; at least 750; at least 1,000; at least 1,500; at least 2,000; at least 5,000; at least 10,000; at least 20,000; at least 50,000; at least 100,000; at least 500,000; at least 1,000,000; at least 10,000,000 or more compounds) are screened according to the method disclosed herein.

The screening method may also be used to generate libraries for reference, further research, or therapeutic applications, which library can be constructed by use of the high throughput nature of the screening method on a selection of compounds. In some embodiments, the disclosure relates to a library of compounds, wherein each compound in the library comprises a common functional group identified according to the method of screening a plurality of compounds. In some embodiments, the library comprises at least 2 compounds (e.g., at least 2; at least 3; at least 4; at least 5; at least 6; at least 7; at least 8; at least 9; at least 10; at least 11; at least 12; at least 13; at least 14; at least 15; at least 16; at least 17; at least 18; at least 19; at least 20; at least 25; at least 30; at least 35; at least 40; at least 45; at least 50; at least 60; at least 70; at least 80; at least 90; at least 100; at least 125; at least 150; at least 175; at least 200; at least 300; at least 400; at least 500; at least 750; at least 1,000; at least 1,500; at least 2,000; at least 5,000; at least 10,000; at least 20,000; at least 50,000; at least 100,000; at least 500,000; at least 1,000,000; at least 10,000,000 or more compounds). In some embodiments the library comprises, a plurality of common functional groups. In some embodiments, the library comprises at least 2 common function groups (e.g., at least 2; at least 3; at least 4; at least 5; at least 6; at least 7; at least 8; at least 9; at least 10; at least 11; at least 12; at least 13; at least 14; at least 15; at least 16; at least 17; at least 18; at least 19; at least 20; at least 25; at least 30; at least 35; at least 40; at least 45; at least 50; at least 60; at least 70; at least 80; at least 90; at least 100; at least 125; at least 150; at least 175; at least 200; at least 300; at least 400; at least 500; at least 750; at least 1,000; at least 1,500; at least 2,000; at least 5,000; at least 10,000; at least 20,000; at least 50,000; at least 100,000; at least 500,000; at least 1,000,000; at least 10,000,000 or more common functional groups). In some embodiments, each common function group in the library comprises, at least 2 compounds (e.g., at least 2; at least 3; at least 4; at least 5; at least 6; at least 7; at least 8; at least 9; at least 10; at least 11; at least 12; at least 13; at least 14; at least 15; at least 16; at least 17; at least 18; at least 19; at least 20; at least 25; at least 30; at least 35; at least 40; at least 45; at least 50; at least 60; at least 70; at least 80; at least 90; at least 100; at least 125; at least 150; at least 175; at least 200; at least 300; at least 400; at least 500; at least 750; at least 1,000; at least 1,500; at least 2,000; at least 5,000; at least 10,000; at least 20,000; at least 50,000; at least 100,000; at least 500,000; at least 1,000,000; at least 10,000,000 or more compounds).

Any subject requiring modulation of PU.1 expression or activity, as well as any subject at risk of having, suspected of having, or having a disorder related to PU.1 expression or activity may require PU.1 modulation for treatment. Accordingly, in another aspect, the disclosure relates to a method of treating a disorder in a subject, comprising: (a) identifying a subject who has, is at risk of having, or is suspected of having a disorder related to PU.1 expression; and (b) administering an effective amount of at least one compound identified as an inhibitor of PU.1 by the methods described herein.

The term “effective amount,” as used herein, refers to an amount of a biologically active agent (e.g., a compound identified as a PU.1 inhibitor) that is sufficient to elicit a desired biological response. For example, in some embodiments, an effective amount of a PU.1 inhibitor may refer to the amount of the inhibitor sufficient to inhibit PU.1 activity (e.g., block PU.1 from binding to a lambda beta binding site and thus influencing gene expression) in a subject to facilitate a specific result (e.g., treat a disorder related to PU.1 expression). In some embodiments, an effective amount of a PU.1 inhibitor as provided herein may refer to the amount of the PU.1 inhibitor sufficient to treat Alzheimer's disease (AD). As will be appreciated by the skilled artisan, the effective amount of an PU.1 inhibitor (e.g., a PU.1 inhibitor identified as a result of the methods herein) may vary depending on various factors as, for example, on the desired biological response, e.g., on the specific gene for which expression is being modulated, on the cell or tissue being targeted, and on the PU.1 inhibitor being used.

In some embodiments, the amount of a PU.1 inhibitor reduces PU.1 activity by at least 1%. In some embodiments, the amount of a PU.1 inhibitor reduces PU.1 activity by at least 10%. In some embodiments, the amount of a PU.1 inhibitor reduces PU.1 activity by at least 20%. In some embodiments, the amount of a PU.1 inhibitor reduces PU.1 activity by at least 30%. In some embodiments, the amount of a PU.1 inhibitor reduces PU.1 activity by at least 40%. In some embodiments, the amount of a PU.1 inhibitor reduces PU.1 activity by at least 50%. In some embodiments, the amount of a PU.1 inhibitor reduces PU.1 activity by at least 60%. In some embodiments, the amount of a PU.1 inhibitor reduces PU.1 activity by at least 70%. In some embodiments, the amount of a PU.1 inhibitor reduces PU.1 activity by at least 80%. In some embodiments, the amount of a PU.1 inhibitor reduces PU.1 activity by at least 90%. In some embodiments, the amount of a PU.1 inhibitor reduces PU.1 activity by more than 90%. PU.1 activity may be measured or assessed by an technique known in the art. Appropriate methods and techniques will be readily appreciated by the skilled artisan.

Any number of compounds may be administered as part of the methods of treatment disclosed herein, both PU.1 inhibitors, and combinations of PU.1 inhibitors in connection with other compounds. In some embodiments, at least one (e.g., at least 2; at least 3; at least 4; at least 5; at least 6; at least 7; at least 8; at least 9; at least 10; at least 11; at least 12; at least 13; at least 14; at least 15; at least 16; at least 17; at least 18; at least 19; at least 20 or more) compound identified as an inhibitor of PU.1 is administered. In some embodiments, the at least one compound identified as an inhibitor of PU.1 is selected from Table 1. In some embodiments, the at least one compound identified as an inhibitor of PU.1 is A11:

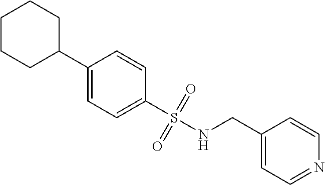 [SMILES: O=S(=O)(NCc1ccncc1)c1ccc(C2CCCCC2)cc1]

and pharmaceutically acceptable salts, hydrates, solvates, polymorphs, co-crystals, tautomers, stereoisomers, isotopically labeled derivatives, and prodrugs thereof.

In some embodiments, the at least one compound identified as an inhibitor of PU.1 administered is of Formula V:

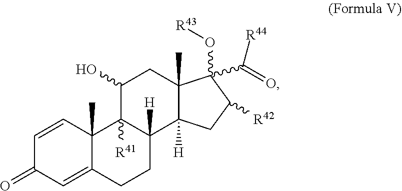

or a pharmaceutically acceptable salt, solvate, hydrate, polymorph, co-crystal, tautomer, stereoisomer, isotopically labeled derivative, or prodrug thereof, wherein:

R⁴¹ is hydrogen or halogen;

R⁴² is hydrogen or substituted or unsubstituted alkyl;

R⁴³ is hydrogen, —C(═O)R^{e}, or —C(═O)OR^{e};

each instance of R^{e} is independently substituted or unsubstituted alkyl or substituted or unsubstituted heteroaryl; and

R⁴⁴ is substituted or unsubstituted alkyl or —OR^{e}.

In certain embodiments, R⁴² is hydrogen or unsubstituted C₁₋₆ alkyl. In certain embodiments, R⁴⁴ is C₁₋₆ alkyl substituted with at least one halogen or at least one —OH, or is —OR^{e}. In certain embodiments, each instance of R^{e} is independently unsubstituted C₁₋₆ alkyl, C₁₋₆ alkyl substituted with at least one halogen, or substituted or unsubstituted furanyl.

In some embodiments, the at least one compound identified as an inhibitor of PU.1 administered is of the formula:

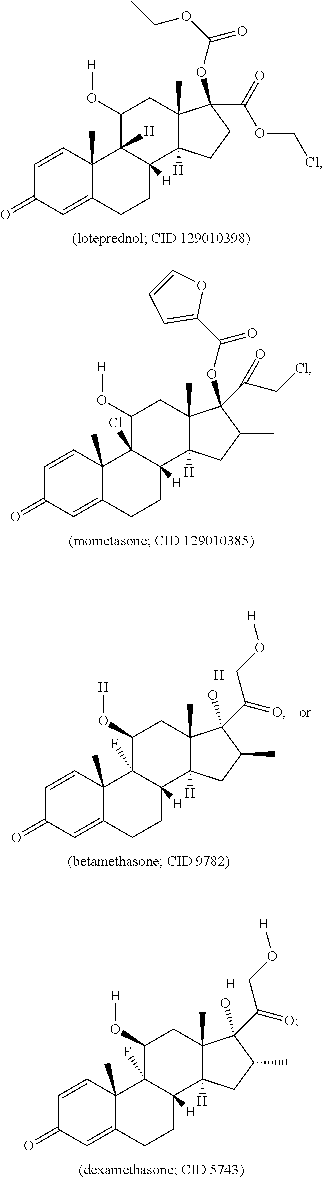

or a pharmaceutically acceptable salt, solvate, hydrate, polymorph, co-crystal, tautomer, stereoisomer, isotopically labeled derivative, or prodrug thereof.

In some embodiments, the at least one compound identified as an inhibitor of PU.1 administered is a compound (additional compound) of the formula:

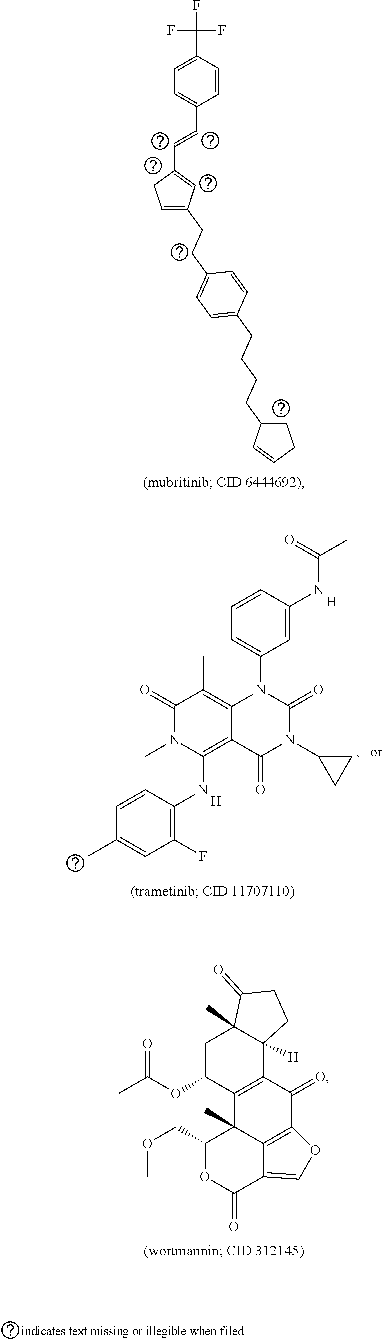

or a pharmaceutically acceptable salt, solvate, hydrate, polymorph, co-crystal, tautomer, stereoisomer, isotopically labeled derivative, or prodrug thereof.

In yet another aspect, the present invention provides kits (e.g., pharmaceutical packs) comprising a compound described herein, or a pharmaceutically acceptable salt, hydrate, solvate, polymorph, co-crystal, tautomer, stereoisomer, isotopically labeled derivative, or prodrug thereof, or a pharmaceutical composition thereof. In certain embodiments, the kit comprises an addition therapeutic agent. In certain embodiments, the kit further comprises instructions for use or administration.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33: 2725 (1977); Eliel, E. L. Stereochemistry of Carbon Compounds (McGraw-Hill, N.Y., 1962); and Wilen, S. H., Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of 19F with 18F, or the replacement of 12C with 13C or 14C are within the scope of the disclosure. Such compounds are useful, for example, as analytical tools or probes in biological assays.

In some embodiments, at least two compounds are administered, wherein at least one of which is identified as an inhibitor of PU.1. In some embodiments, at least two compounds are administered, wherein at least one of which is identified as an inhibitor of PU.1 is selected from Table 1.

In some embodiments, the method further comprises, (c) administering at least one additional compound, prior to, contemporaneously with, or subsequent to, the administration of the at least one compound identified as an inhibitor of PU.1. For example, the compound may be administered within the hour prior, within the same day, within the previous day, or within any period of time prior to the administration of the at least one compound identified as an inhibitor of PU.1 in an effort to increase the efficacy of the treatment including the at least one compound identified as an inhibitor of PU.1. Similarly, the compound may be administered within a few minutes before or after, or contemporaneously with (e.g., injected or administered at the same time, or formulated in the same dose), or within any period of time prior to the administration of the at least one compound identified as an inhibitor of PU.1 in an effort to increase the efficacy of the treatment including the at least one compound identified as an inhibitor of PU.1. Further yet, similarly, within the hour after, within the same day, within the following day, or within any period of time after the administration of the at least one compound identified as an inhibitor of PU.1 in an effort to increase the efficacy of the treatment including the at least one compound identified as an inhibitor of PU.1. In some embodiments, the at least one additional compound is selected from the group comprising: an amyloid antibody or cognitive enhancer. In some embodiments, the at least one additional compound is an amyloid antibody. In some embodiments, the at least one additional compound is a cognitive enhancer.

In some embodiments, the disorder is a disorder related to, or affected by, PU.1 expression or repression. In some embodiments, the disorder is related to abnormal PU.1 expression. In some embodiments, the disorder is selected from the group comprising: multiple sclerosis, Parkinson's Disease, Huntington's Disease, amyotrophic lateral sclerosis, neuroinflammation, frontotemporal dementia, dementia with Lewy bodies, neuropathic pain, inflammatory pain, neuropathic itch, inflammatory itch, neuropathic dysesthesia, inflammatory dysesthesia, dementia, glioma, brain tumors, Batten disease, Down's Syndrome, Nasu-Hakola, prion disease, Cockayne syndrome, Ataxia—telangiectasia, xeroderma pigmentosum, schizophrenia, bipolar disorder, epilepsy, motor neuron disease, sciatica, Friedreich's ataxia, Gerstmann—Straussler—Scheinker Disease, Kuru, Alper's Disease, apnea, corticobasal degeneration, Leigh's Disease, Monomelic amyotrophy, multiple system atrophy, multiple system atrophy with orthostatic hypotension, narcolepsy, neurodegeneration with brain iron accumulation, opsoclonus myoclonus, progressive multifocal leukoencephalopathy, strationigral degeneration, transmissible spongiform encephalopathis, ataxia, Sjogren's disease, Sandhoff disease, Myasthenia gravis, Tay—Sachs disease, neuronal ceroid lipofuscinosis, senesence, progeria, sepsis, Lyme disease, leukemia, lupus, fibrosis, cancer, hematologic cancer, bone cancer, glioblastomas, inflammatory diseases, inflammatory disorders, autoimmune disorders, endotoxemia and neurodegenerative diseases, including without limitation, such conditions ase acute myeloid leukemia, rheumatoid arthritis, contact dermatitis, asthma, inflammatory bowel disease, pediatric atrophy, giant cell arteritis, Alzheimer's disease, and systemic lupus. In some embodiments, the disorder is a disorder related to cells from a hematopoietic lineage. In some embodiments, the disorder is selected from the group comprising: Alzheimer's disease (AD), inflammation, or excessive myelin uptake. In some embodiments, the disorder is AD.

The compositions (e.g., vectors, plasmids, libraries, and/or nucleic acids encoding the various components) as described herein may, in some embodiments be administered in any suitable manner known in the art which will readily be apparent to one of ordinary skill in the art. The compositions (e.g., vectors, plasmids, libraries, and/or nucleic acids encoding the various components) as described herein, may be delivered to a subject (e.g., treatment of a disorder) by: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

The compositions (e.g., vectors, plasmids, libraries, and/or nucleic acids encoding the various components) as described herein may, in some embodiments, be assembled into pharmaceutical, diagnostic, or research kits to facilitate their use in therapeutic, diagnostic, or research applications. A kit may include one or more containers housing the compositions (e.g., vectors, plasmids, libraries, and/or nucleic acids encoding the various components) of the disclosure and instructions for use. Specifically, such kits may include one or more compositions (e.g., vectors, plasmids, libraries, and/or nucleic acids encoding the various components) described herein, along with instructions describing the intended application and the proper use of these compositions (e.g., vectors, plasmids, libraries, and/or nucleic acids encoding the various components). In certain embodiments compositions (e.g., vectors, plasmids, libraries, and/or nucleic acids encoding the various components) in a kit may be in a pharmaceutical formulation and dosage suitable for a particular application and for a method of administration of the compositions (e.g., vectors, plasmids, libraries, and/or nucleic acids encoding the various components). Kits for research purposes may contain the components in appropriate concentrations or quantities for running various experiments.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise proces sable (e.g., to an active form), for example, by the addition of a suitable solvent or other species (for example, water or a cell culture medium), which may or may not be provided with the kit.

As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the compositions (e.g., vectors, plasmids, libraries, and/or nucleic acids encoding the various components) described herein in one or more containers. As an example, in one embodiment, the kit may include instructions for mixing one or more compositions (e.g., vectors, plasmids, libraries, and/or nucleic acids encoding the various components) of the kit and/or isolating and mixing a sample and applying to a subject. The kit may include a container housing compositions (e.g., vectors, plasmids, libraries, and/or nucleic acids encoding the various components) described herein. The compositions (e.g., vectors, plasmids, libraries, and/or nucleic acids encoding the various components) may be in the form of a liquid, gel, or solid (powder). The compositions (e.g., vectors, plasmids, libraries, and/or nucleic acids encoding the various components) may be prepared sterilely, packaged in syringe and shipped refrigerated. Alternatively it may be housed in a vial or other container for storage. A second container may have other agents prepared sterilely. Alternatively the kit may include the active compositions (e.g., vectors, plasmids, libraries, and/or nucleic acids encoding the various components) premixed and shipped in a syringe, vial, tube, or other container. The kit may have one or more or all of the compositions (e.g., vectors, plasmids, libraries, and/or nucleic acids encoding the various components) required to administer the compositions (e.g., vectors, plasmids, libraries, and/or nucleic acids encoding the various components) to an animal, such as a syringe, topical application devices, or intravenous needle, tubing, and bag, particularly in the case of the kits for producing specific somatic animal models.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

Some aspects of this disclosure provide cells comprising any of the compositions (e.g., vectors, plasmids, libraries, and/or nucleic acids encoding the various components) disclosed herein. In some embodiments, a cell is transiently or non-transiently transfected with one or more vectors described herein. In some embodiments, an cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa—S3, Huh1, Huh4, Huh7, HUVEC, HASMC, HEKn, HEKa, MiaPaCell, Panc1, PC-3, TF₁, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO₁, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX₁₀, NCI-H69/LX₂₀, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassus, Va.)). In some embodiments, a cell transfected with one or more compositions (e.g., vectors, plasmids, libraries, and/or nucleic acids encoding the various components) described herein is used to establish a new cell line comprising one or more vector-derived sequences. In some embodiments, a cell transiently transfected with the compositions (e.g., vectors, plasmids, libraries, and/or nucleic acids encoding the various components) as described herein. In some embodiments, cells transiently or non-transiently transfected with one or more compositions (e.g., vectors, plasmids, libraries, and/or nucleic acids encoding the various components) described herein, or cell lines derived from such cells are used in assessing one or more test compounds.

In another aspect, the present disclosure provides pharmaceutical compositions comprising:

a compound of Formula I:

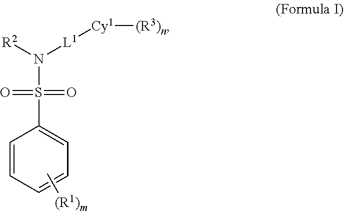

or a pharmaceutically acceptable salt, solvate, hydrate, polymorph, co-crystal, tautomer, stereoisomer, isotopically labeled derivative, or prodrug thereof; and

a pharmaceutically acceptable excipient;

wherein:

each instance of R¹ is independently halogen, substituted or unsubstituted alkyl, —OR^{a}, substituted or unsubstituted carbocyclyl, substituted or unsubstituted heterocyclyl, or substituted or unsubstituted heteroaryl;

or two instances of R¹ are joined with the intervening atoms to form substituted or unsubstituted heterocyclyl or substituted or unsubstituted heteroaryl, and each of the remaining instances of R¹, if present, is independently halogen, substituted or unsubstituted alkyl, or —OR^{a};

each instance of R^{a} is independently substituted or unsubstituted alkyl, substituted or unsubstituted carbocyclyl, or substituted or unsubstituted aryl;

m is 1, 2, or 3;

R² is hydrogen or —C(═O)OR^{a};

L¹ is a single bond or substituted or unsubstituted methylene;

Cy¹ is aryl or heteroaryl;

each instance of R³ is independently halogen, substituted or unsubstituted alkyl, —OR^{a}, —N(R^{a})₂, —C(═O)R^{a}, —C(═O)OR^{a}, or —S(═O)₂R^{a};

or Cy¹ is a single bond, and R² and one instance of R³ are joined with the intervening atoms to form substituted or unsubstituted heterocyclyl or substituted or unsubstituted heteroaryl, and each of the remaining instances of R³, if present, is independently halogen, substituted or unsubstituted alkyl, —OR^{a}, —N(R^{a})₂, —C(═O)R^{a}, —C(═O)OR^{a}, or —S(═O)₂R^{a}; and

n is 0, 1, 2, or 3.

In certain embodiments, at least one instance of R¹ is substituted or unsubstituted carbocyclyl. In certain embodiments, R² is hydrogen. In certain embodiments, L¹ is unsubstituted methylene. In certain embodiments, Cy¹ is pyridyl. In certain embodiments, the compound is of the formula:

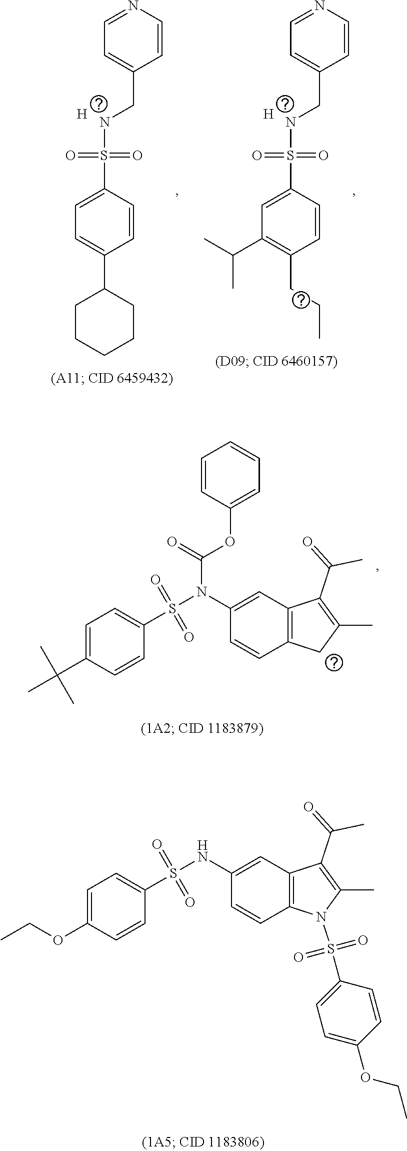

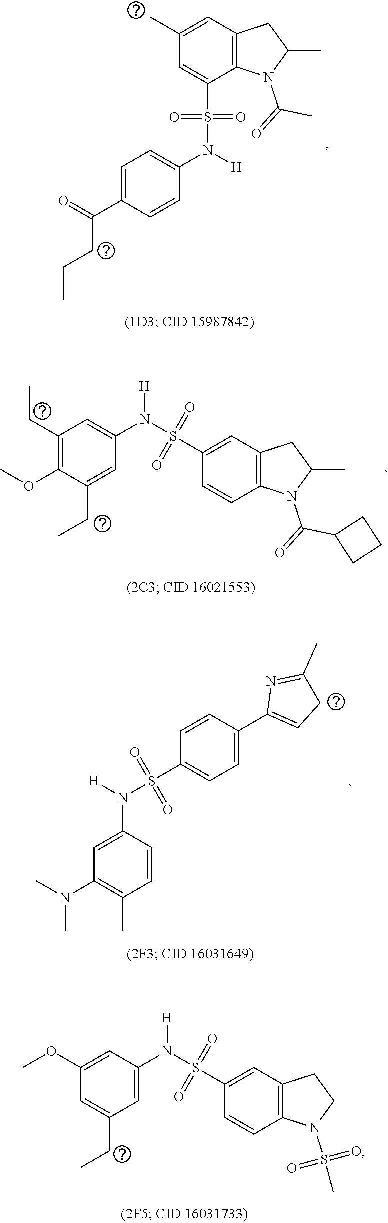

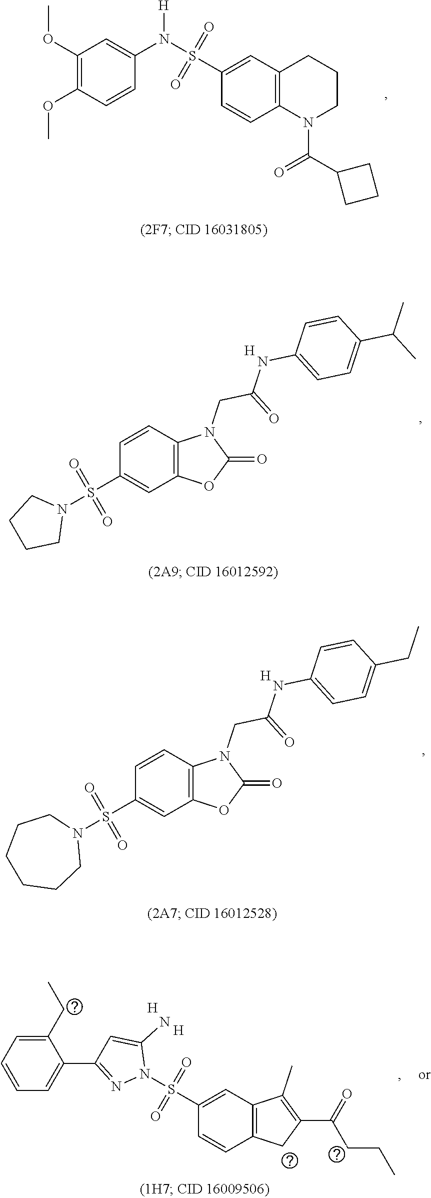

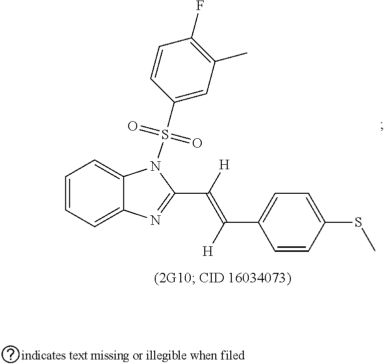 [SMILES: CSc1ccc(/C=C/c2nc3ccccc3n2S(=O)(=O)c2ccc(F)c(C)c2)cc1]

or a pharmaceutically acceptable salt, solvate, hydrate, polymorph, co-crystal, tautomer, stereoisomer, isotopically labeled derivative, or prodrug thereof.

In another aspect, the present disclosure provides pharmaceutical compositions comprising:

a compound of Formula II:

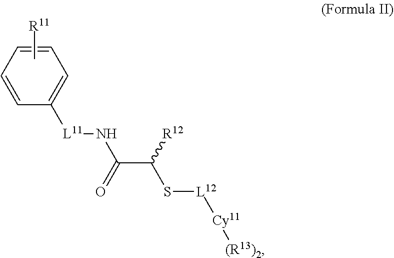

or a pharmaceutically acceptable salt, solvate, hydrate, polymorph, co-crystal, tautomer, stereoisomer, isotopically labeled derivative, or prodrug thereof; and

a pharmaceutically acceptable excipient;

wherein:

R¹¹ is hydrogen, halogen, or substituted or unsubstituted alkyl;

L¹¹ is a single bond, substituted or unsubstituted methylene, or substituted or unsubstituted ethylene;

R12 is hydrogen or substituted or unsubstituted alkyl;

L¹² is a single bond or substituted or unsubstituted methylene;

Cy¹¹ is heteroaryl or heterocyclyl; and

each instance of R¹³ is independently substituted or unsubstituted alkyl, substituted or unsubstituted aryl, or oxo.

In certain embodiments, R¹¹ is hydrogen, halogen, or unsubstituted C₁₋₆ alkyl. In certain embodiments, L¹¹ is a single bond, unsubstituted methylene, or unsubstituted ethylene. In certain embodiments, R¹² is hydrogen or unsubstituted C₁₋₆ alkyl. In certain embodiments, wherein L¹² is a single bond or unsubstituted methylene. In certain embodiments, wherein Cy¹¹ is oxazolyl or

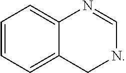

In certain embodiments, each instance of R¹³ is independently unsubstituted C₁₋₆ alkyl, substituted or unsubstituted phenyl, or oxo. In certain embodiments, the compound is of the formula:

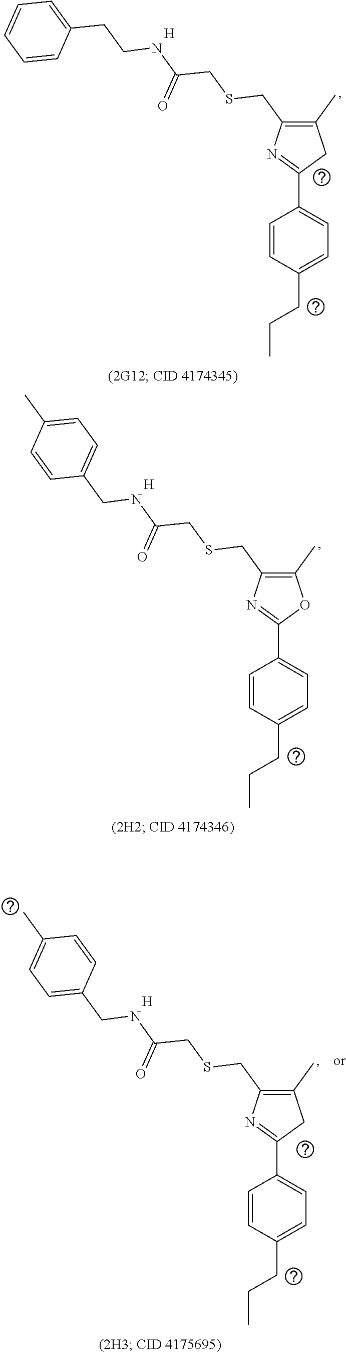

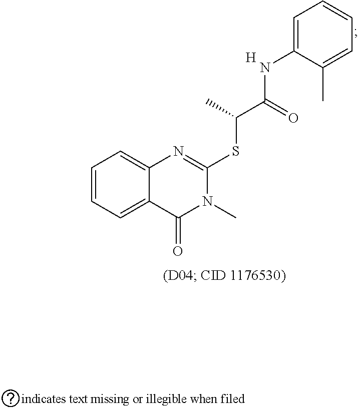

or a pharmaceutically acceptable salt, solvate, hydrate, polymorph, co-crystal, tautomer, stereoisomer, isotopically labeled derivative, or prodrug thereof.

In another aspect, the present disclosure provides pharmaceutical compositions comprising:

a compound of Formula III:

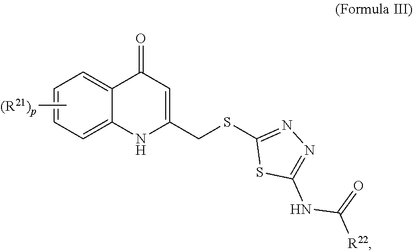

or a pharmaceutically acceptable salt, solvate, hydrate, polymorph, co-crystal, tautomer, stereoisomer, isotopically labeled derivative, or prodrug thereof; and

a pharmaceutically acceptable excipient;

wherein:

each instance of R²¹ is substituted or unsubstituted alkyl;

p is 1 or 2; and

R²² is substituted or unsubstituted alkyl or substituted or unsubstituted aryl.

In certain embodiments, each instance of R²¹ is unsubstituted C₁₋₆ alkyl. In certain embodiments, R²² is unsubstituted C₁₋₆ alkyl or substituted or unsubstituted phenyl. In certain embodiments, the compound is of the formula:

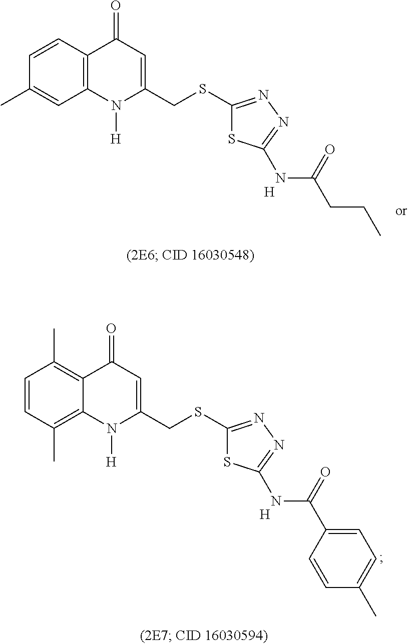

or a pharmaceutically acceptable salt, solvate, hydrate, polymorph, co-crystal, tautomer, stereoisomer, isotopically labeled derivative, or prodrug thereof.

In another aspect, the present disclosure provides pharmaceutical compositions comprising:

a compound of Formula IV:

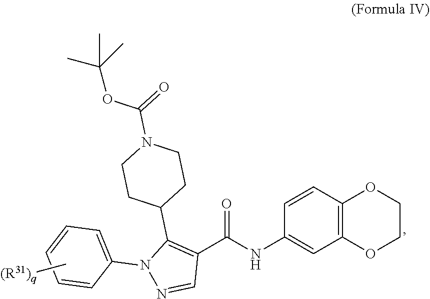

or a pharmaceutically acceptable salt, solvate, hydrate, polymorph, co-crystal, tautomer, stereoisomer, isotopically labeled derivative, or prodrug thereof; and

a pharmaceutically acceptable excipient;

wherein:

each instance of R³¹ is independently halogen or substituted or unsubstituted alkyl;

and

q is 0, 1, or 2.

In certain embodiments, each instance of R³¹ is independently halogen or unsubstituted C₁₋₆ alkyl. In certain embodiments, the compound is of the formula:

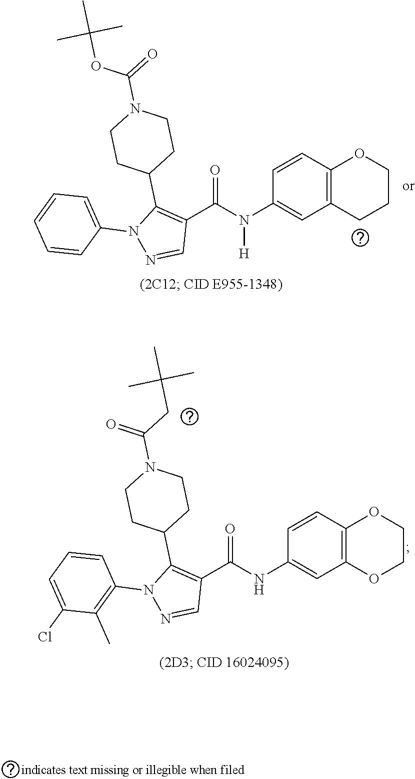 [SMILES: CC(C)(C)OC(=O)N1CCC(c2c(C(=O)Nc3ccc4c(c3)CCCO4)cnn2-c2ccccc2)CC1 ; Cc1c(Cl)cccc1-n1ncc(C(=O)Nc2ccc3c(c2)OCCO3)c1C1CCN(C(=O)CC(C)(C)C)CC1]

or a pharmaceutically acceptable salt, solvate, hydrate, polymorph, co-crystal, tautomer, stereoisomer, isotopically labeled derivative, or prodrug thereof.

In another aspect, the present disclosure provides pharmaceutical compositions (additional pharmaceutical compositions) comprising:

a compound of the formula:

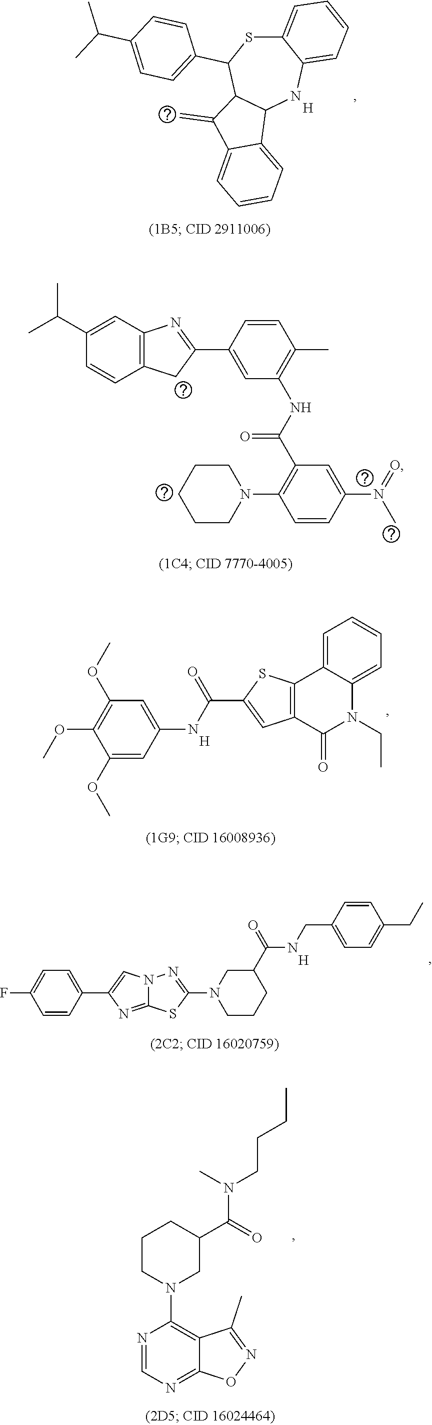

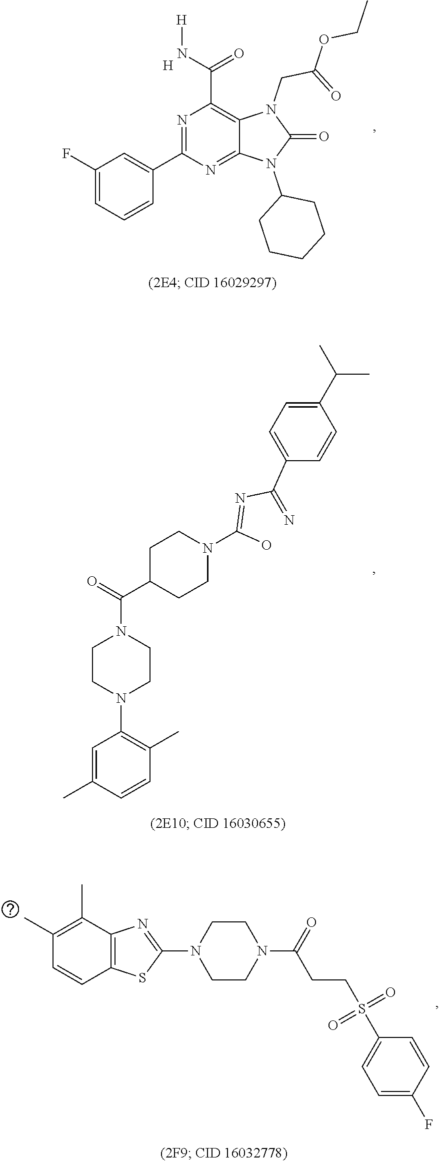

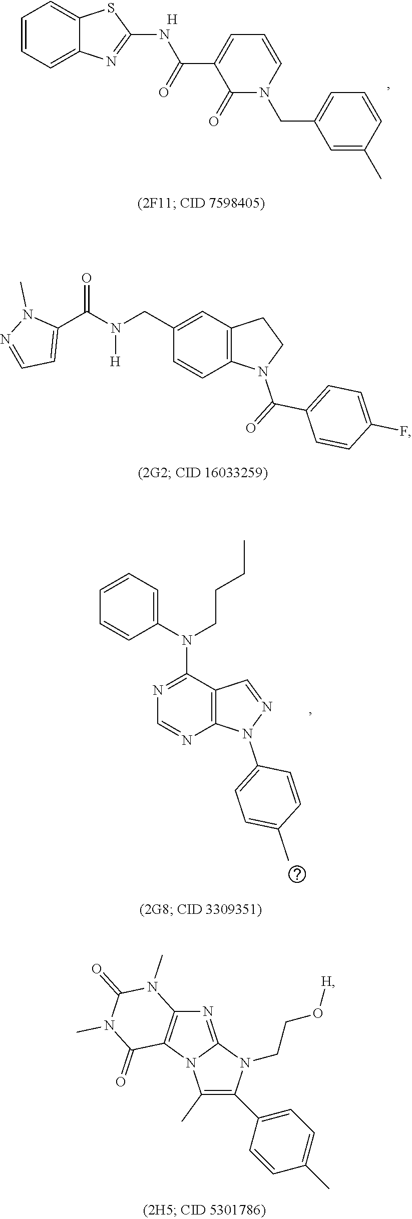

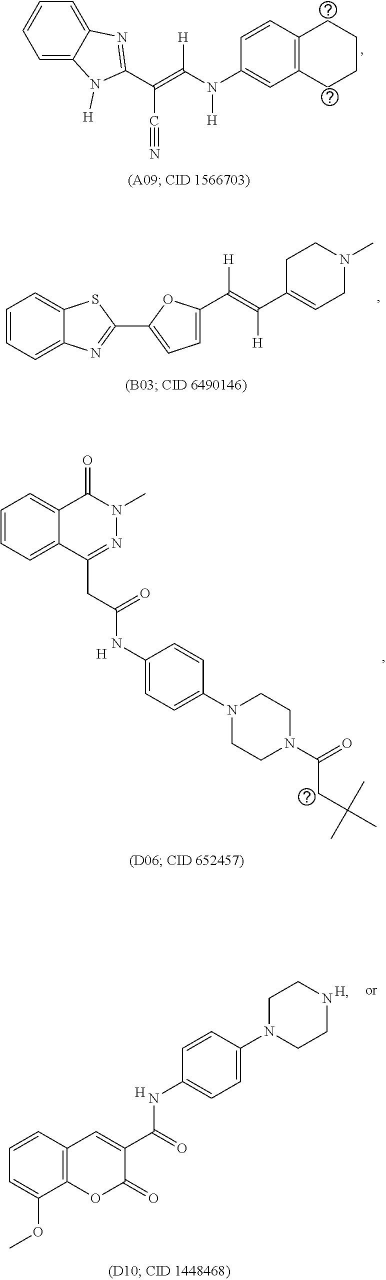

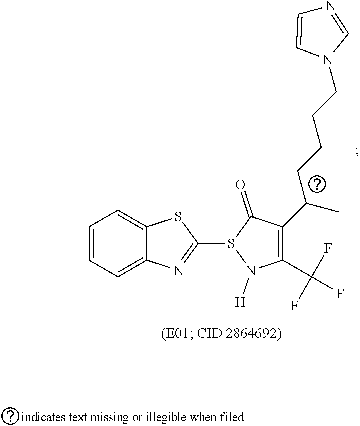

or a pharmaceutically acceptable salt, solvate, hydrate, polymorph, co-crystal, tautomer, stereoisomer, isotopically labeled derivative, or prodrug thereof; and

a pharmaceutically acceptable excipient.

In certain embodiments, at least one pharmaceutically acceptable excipient is not water.

Definitions of specific functional groups and chemical terms are described in more detail below. The chemical elements are identified in accordance with the Periodic Table of the Elements, CAS version, Handbook of Chemistry and Physics, 75^{±}Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC), supercritical fluid chromatography (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33: 2725 (1977); Eliel, Stereochemistry of Carbon Compounds (McGraw-Hill, N.Y., 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond.

Unless otherwise provided, a formula depicted herein includes compounds that do not include isotopically enriched atoms and also compounds that include isotopically enriched atoms. Compounds that include isotopically enriched atoms may be useful as, for example, analytical tools, and/or probes in biological assays.

The term “aliphatic” includes both saturated and unsaturated, nonaromatic, straight chain (i.e., unbranched), branched, acyclic, and cyclic (i.e., carbocyclic) hydrocarbons. In some embodiments, an aliphatic group is optionally substituted with one or more functional groups (e.g., halo, such as fluorine). As will be appreciated by one of ordinary skill in the art, “aliphatic” is intended herein to include alkyl, alkenyl, alkynyl, cycloalkyl, cycloalkenyl, and cycloalkynyl moieties.

When a range of values (“range”) is listed, it is intended to encompass each value and sub-range within the range. A range is inclusive of the values at the two ends of the range unless otherwise provided. For example, “an integer between 1 and 4” refers to 1, 2, 3, and 4. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅-6 alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkylyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₁₀ alkylalkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n—heptyl (C₇), n-octyl (C₈) and the like. Unless otherwise specified, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₂ alkyl (e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)). The attachment point of alkyl may be a single bond (e.g., as in —CH₃), double bond (e.g., as in ═CH₂), or triple bond (e.g., as in ≡CH). The moieties ═CH₂ and ≡CH are also alkyl.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 20 carbon atoms, one or more (e.g., two, three, or four, as valency permits) carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃ or

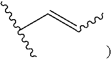

may be in the (E)- or (Z)-configuration.

“Alkynyl” refers to a radical of a straight-chain or branched hydrocarbon group having from 2 to 20 carbon atoms, one or more (e.g., two, three, or four, as valency permits) carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 13 ring carbon atoms (“C₃₋₁₃ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged, or spiro ring system such as a bicyclic system (“bicyclic carbocyclyl”). Carbocyclyl can be saturated, and saturated carbocyclyl is referred to as “cycloalkyl.” In some embodiments, carbocyclyl is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅-6 cycloalkyl”). In some embodiments, a cycloalkyl group has 5 to 10 ring carbon atoms (“C5-10 cycloalkyl”). Examples of C₅-6 cycloalkyl groups include cyclopentyl (C₅) and cyclohexyl (C₅). Examples of C₃₋₆ cycloalkyl groups include the aforementioned C₅-6 cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl. Carbocyclyl can be partially unsaturated. Carbocyclyl may include zero, one, or more (e.g., two, three, or four, as valency permits) C═C double bonds in all the rings of the carbocyclic ring system that are not aromatic or heteroaromatic. Carbocyclyl including one or more (e.g., two or three, as valency permits) C═C double bonds in the carbocyclic ring is referred to as “cycloalkenyl.” Carbocyclyl including one or more (e.g., two or three, as valency permits) C≡C triple bonds in the carbocyclic ring is referred to as “cycloalkynyl.” Carbocyclyl includes aryl. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl. In certain embodiments, the carbocyclyl is substituted or unsubstituted, 3-to 7-membered, and monocyclic. In certain embodiments, the carbocyclyl is substituted or unsubstituted, 5- to 13-membered, and bicyclic. In certain embodiments, the carbocyclyl is substituted or unsubstituted, 6-to 13-membered, and tricyclic.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅-6 cycloalkyl”). In some embodiments, a cycloalkyl group has 5 to 10 ring carbon atoms (“C5-10 cycloalkyl”). Examples of C₅-6 cycloalkyl groups include cyclopentyl (C₅) and cyclohexyl (C₅). Examples of C₃₋₆ cycloalkyl groups include the aforementioned C₅-6 cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 13-membered non-aromatic ring system having ring carbon atoms and 1 to 4 ring heteroatoms, wherein each heteroatom is independently selected from nitrogen, oxygen, and sulfur (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system such as a bicyclic system (“bicyclic heterocyclyl”). A heterocyclyl group can be saturated or can be partially unsaturated. Heterocyclyl may include zero, one, or more (e.g., two, three, or four, as valency permits) double bonds in all the rings of the heterocyclic ring system that are not aromatic or heteroaromatic. Partially unsaturated heterocyclyl groups includes heteroaryl. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl. In certain embodiments, the heterocyclyl is substituted or unsubstituted, 3-to 7-membered, and monocyclic. In certain embodiments, the heterocyclyl is substituted or unsubstituted, 5-to 13-membered, and bicyclic. In certain embodiments, the heterocyclyl is substituted or unsubstituted, 6-to 13-membered, and tricyclic.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include azirdinyl, oxiranyl, or thiiranyl. Exemplary 4-membered heterocyclyl groups containing one heteroatom include azetidinyl, oxetanyl and thietanyl. Exemplary 5-membered heterocyclyl groups containing one heteroatom include tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include dioxolanyl, oxasulfuranyl, disulfuranyl, and oxazolidin-2-one. Exemplary 5-membered heterocyclyl groups containing three heteroatoms include triazolinyl, oxadiazolinyl, and thiadiazolinyl. Exemplary 6-membered heterocyclyl groups containing one heteroatom include piperidinyl, tetrahydropyranyl, dihydropyridinyl, and thianyl. Exemplary 6-membered heterocyclyl groups containing two heteroatoms include piperazinyl, morpholinyl, dithianyl, and dioxanyl. Exemplary 6-membered heterocyclyl groups containing two heteroatoms include triazinanyl. Exemplary 7-membered heterocyclyl groups containing one heteroatom include azepanyl, oxepanyl and thiepanyl. Exemplary 8-membered heterocyclyl groups containing one heteroatom include azocanyl, oxecanyl, and thiocanyl. Exemplary 5-membered heterocyclyl groups fused to a C₆ aryl ring (also referred to herein as a 5,6-bicyclic heterocyclic ring) include indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include pyrrolyl, furanyl and thiophenyl. Exemplary 5-membered heteroaryl groups containing two heteroatoms include imidazolyl, pyrazolyl, oxazolyl, isoxazolyl, thiazolyl, and isothiazolyl. Exemplary 5-membered heteroaryl groups containing three heteroatoms include triazolyl, oxadiazolyl, and thiadiazolyl. Exemplary 5-membered heteroaryl groups containing four heteroatoms include tetrazolyl. Exemplary 6-membered heteroaryl groups containing one heteroatom include pyridinyl. Exemplary 6-membered heteroaryl groups containing two heteroatoms include pyridazinyl, pyrimidinyl, and pyrazinyl. Exemplary 6-membered heteroaryl groups containing three or four heteroatoms include triazinyl and tetrazinyl, respectively. Exemplary 7-membered heteroaryl groups containing one heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, aliphatic, alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include halogen, —CN, —NO₂, —N₃, —SO₂H, —SO₃H, —OH, —OR^{aa}, —ON(R^{bb})₂, —N(R^{bb})_{2, —N(R}^{bb})₃⁺X⁻, —N(OR^{cc})R^{bb}, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})4, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄alky, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄alky, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{cc} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of e is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C6-10 aryl and 5-10 membered heteroaryl, or two R^{if} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl) ⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH2, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH2, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl), —OC(NH)NH(C₁₋₆ alkyl), —OC(NH)NH₂, —NHC(NH)N(C₁₋₆ alkyl)₂, —NHC(═NH)NH2, —NHSO₂(C₁₋₆ alkyl), —SO₂N(C₁₋₆ alkyl), —SO₂NH(C₁₋₆ alkyl), —SO₂NH2, —SO₂C₁₋₆ alkyl, —S020C₁₋₆ alkyl, —OSO₂C₁₋₆ alkyl, —SOC₁₋₆ alkyl, —Si(C₁₋₆ alkyl)3, —OSi(C₁₋₆ alkyl)₃ —C(═S)N(C₁₋₆ alkyl)₂, C(═S)NH(C₁₋₆ alkyl), C(═S)NH2, —C(═O)S(C₁₋₆ alkyl), —C(═S)SC₁₋₆ alkyl, —SC(═S)SC₁₋₆ alkyl, —P(═O)(OC₁₋₆ alkyl), —P(═O)(C₁₋₆ alkyl), —OP(═O)(C₁₋₆ alkyl), —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, the carbon atom substituents are independently halogen, substituted (e.g., substituted with one or more halogen moieties) or unsubstituted C₁₋₆ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S2O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₆ alkyl, or an oxygen protecting group when attached to an oxygen atom;

and each R^{bb} is independently hydrogen, substituted (e.g., substituted with one or more halogen) or unsubstituted C₁₋₆ alkyl, or a nitrogen protecting group. In certain embodiments, the nitrogen atom substituents are independently substituted (e.g., substituted with one or more halogen) or unsubstituted C₁₋₆ alkyl or a nitrogen protecting group.

In certain embodiments, the substituent present on a nitrogen atom is a nitrogen protecting group (also referred to as an amino protecting group). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} and groups, and wherein R^{aa}, R^{bb}, R_{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-nitrophenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N ,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2—phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STAB ASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N—methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-cliphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, a nitrogen protecting group is Bn, Boc, Cbz, Fmoc, trifluoroacetyl, triphenylmethyl, acetyl, or Ts.

In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group. In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the oxygen atom substituents are independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃^{±}X⁻, —P(OR″)₂, —P(OR″)₃^{±}X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S—dioxide, 1[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, an oxygen protecting group is silyl, TBDPS, TBDMS, TIPS, TES, TMS, MOM, THP, t-Bu, Bn, allyl, acetyl, pivaloyl, or benzoyl.

In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group. In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the sulfur atom substituents are independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). Sulfur protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

The “molecular weight” of —R, wherein —R is any monovalent moiety, is calculated by substracting the atomic weight of a hydrogen atom from the molecular weight of the molecule R—H. The “molecular weight” of -L-, wherein -L- is any divalent moiety, is calculated by substracting the combined atomic weight of two hydrogen atoms from the molecular weight of the molecule H-L-H.

In certain embodiments, the molecular weight of a substituent is lower than 200, lower than 150, lower than 100, lower than 50, or lower than 25 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, and/or iodine atoms. In certain embodiments, a substituent consists of carbon, hydrogen, and/or fluorine atoms. In certain embodiments, a substituent does not comprise one or more, two or more, or three or more hydrogen bond donors. In certain embodiments, a substituent does not comprise one or more, two or more, or three or more hydrogen bond acceptors.

### EXAMPLES

### Summary

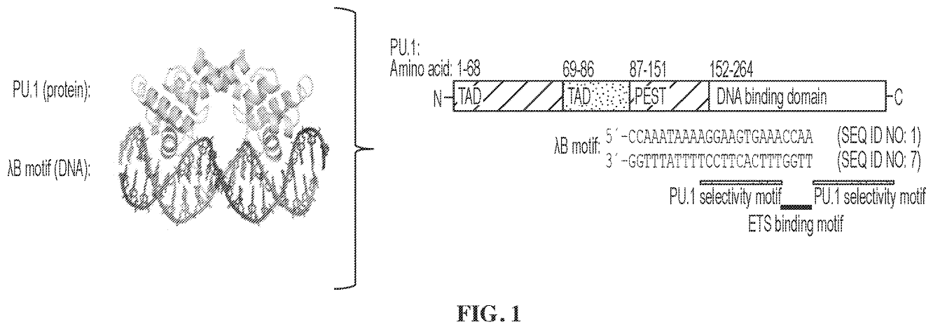

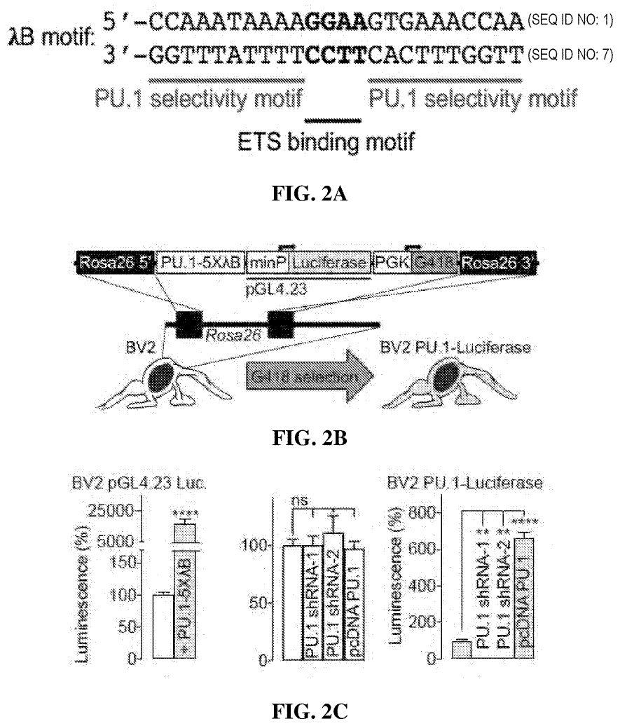

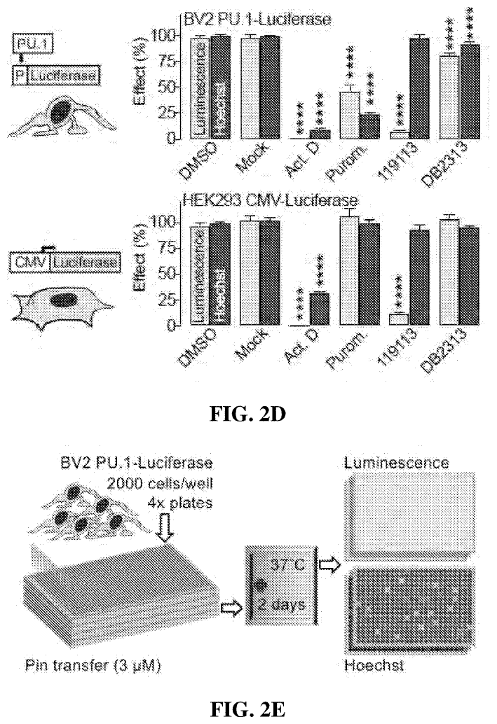

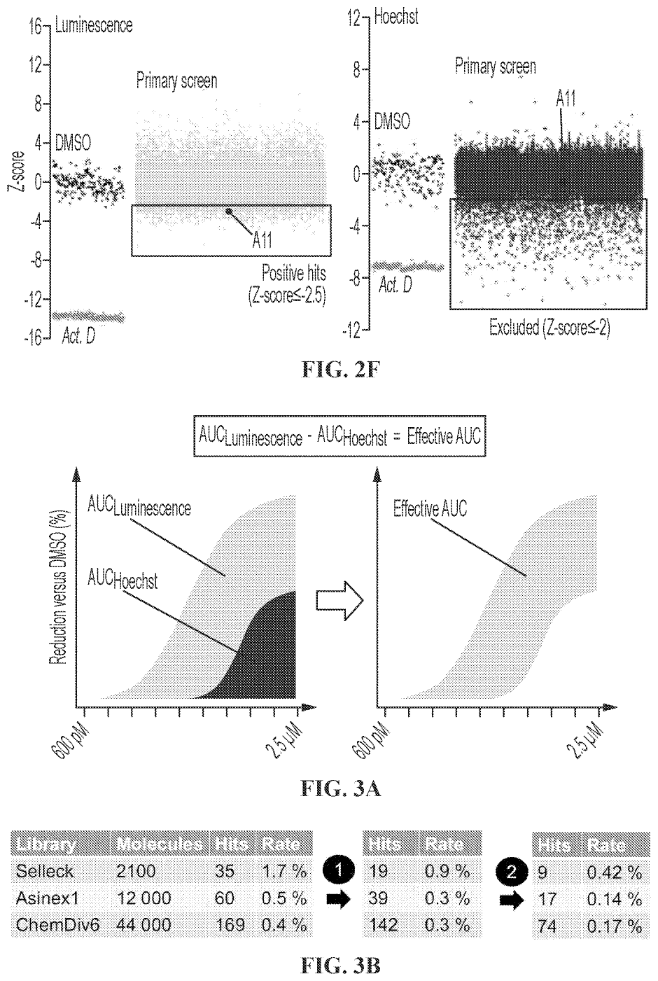

This disclosure relates to the composition of a reporter cell line, the BV2 PU.1-Luciferase cell line. This stable cell line expresses the firefly luciferase reporter under the control of PU.1. This line was created by inserting a previously described PU.1 binding motif called the lambda-beta binding motif (also referred to herein as the “LBB motif”) into the pGL4.23 luciferase plasmid (Munde et al., 2014), (see FIG. 1 and FIG. 2A).

PU.1 belongs to a group of transcription factors that all bind to the middle 5′-GGAA-3′ sequence. The flanking A/T rich sequence is crucial for conferring PU.1 selective binding. The flanking sequence requires unique structural properties of PU.1 and can therefore be targeted by drugs acting as allosteric inhibitors that prevent PU.1 from binding to the LBB. In order to achieve robust PU.1 binding, five tandem copies of the LBBs (5×LBB) were inserted into the luciferase promoter of the pGL4.23 plasmid. The region containing the five LBBs (5×LBB) was then subcloned into the pROSA26-1 plasmid (together with a PGKneo bpA plasmid for Neomycin/G418 resistance) that allows stable integration in chromosome 6 of the immortalized mouse microglial BV2 cell line (FIG. 2B). The stable cell line was created by selection with G418.

### Luciferase Assay

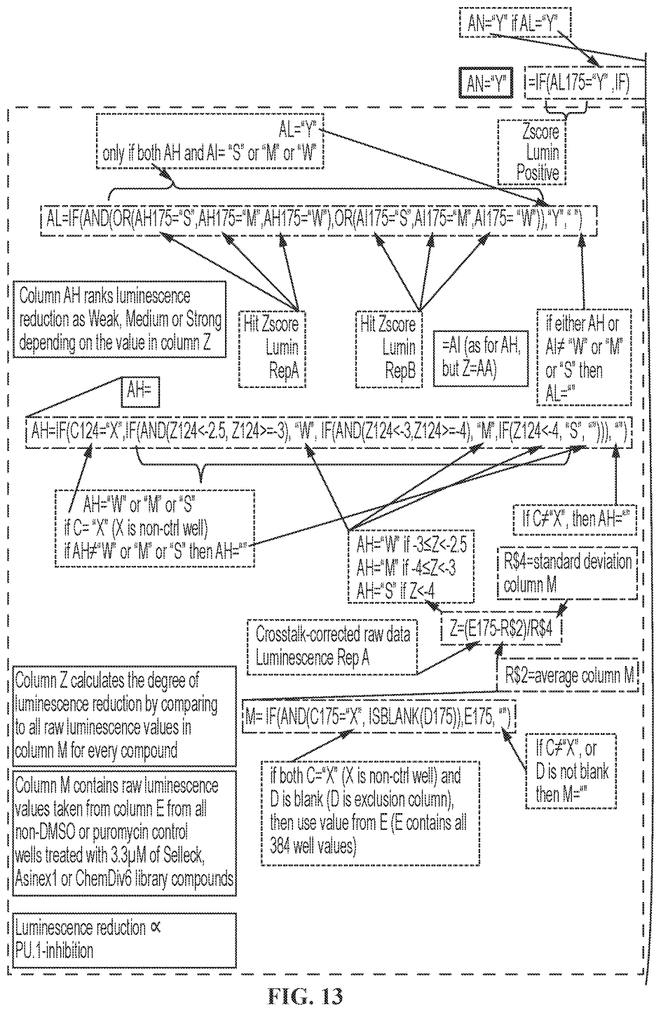

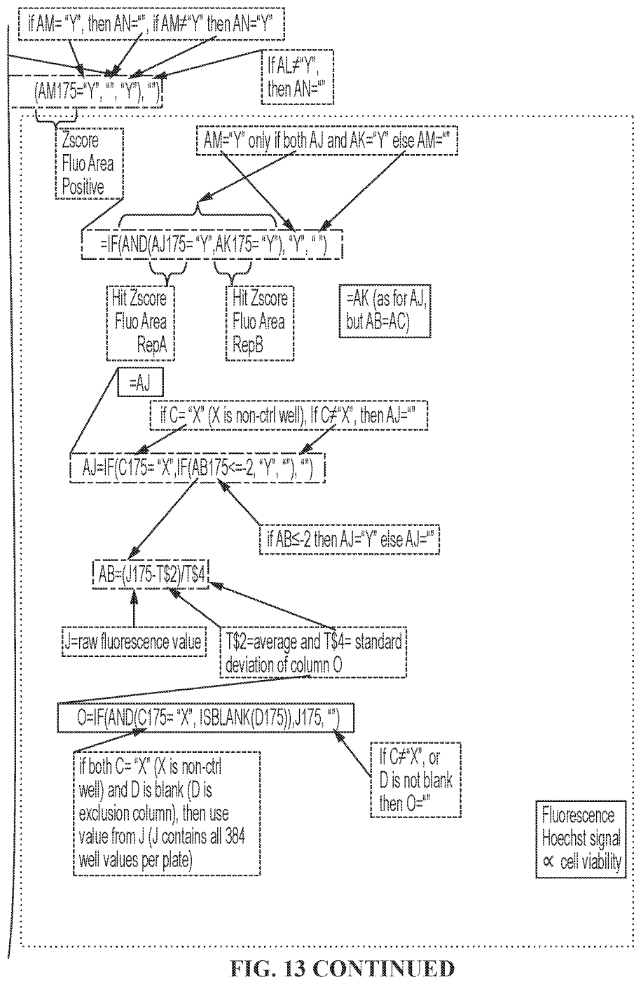

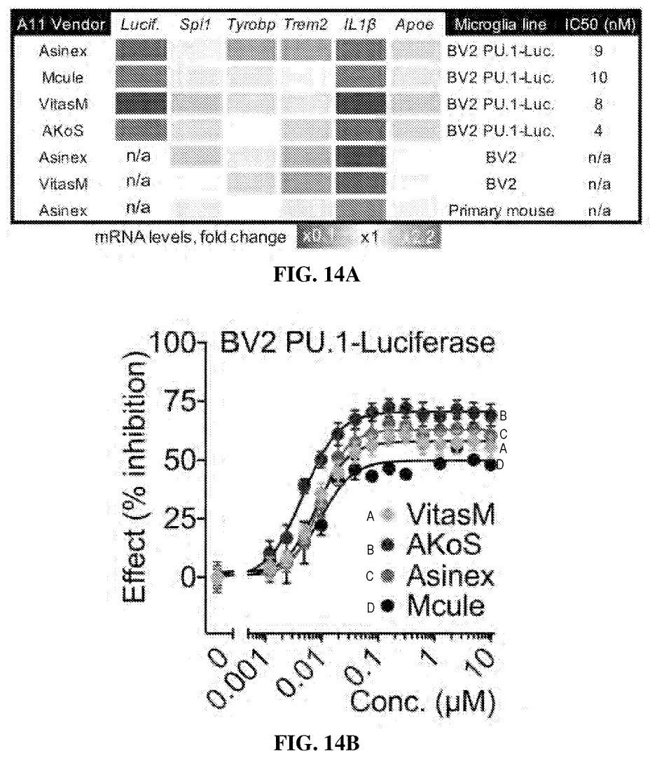

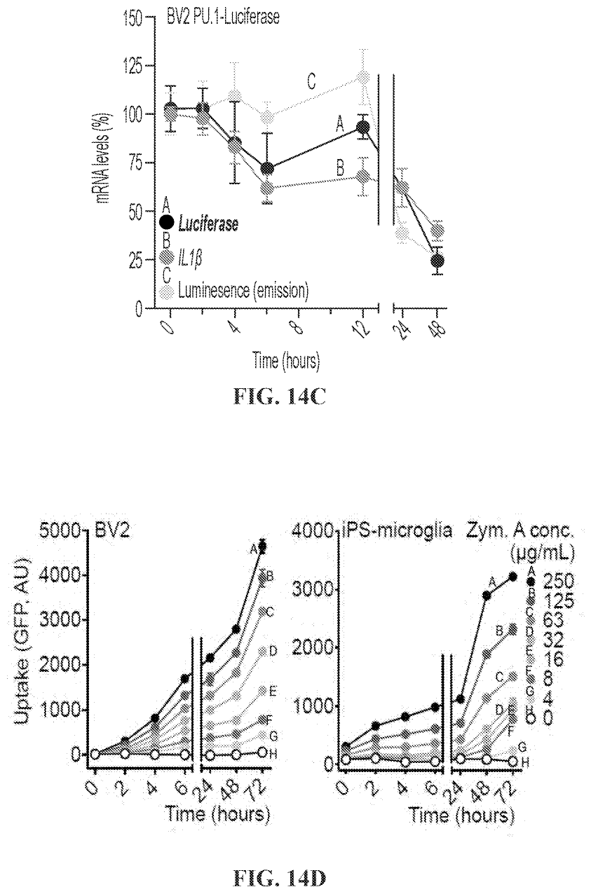

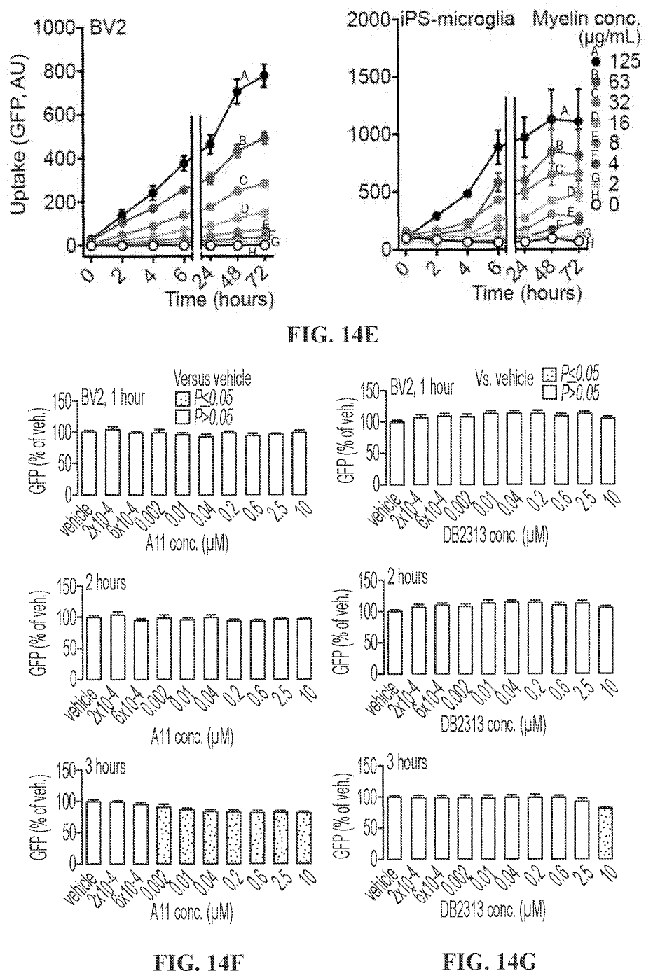

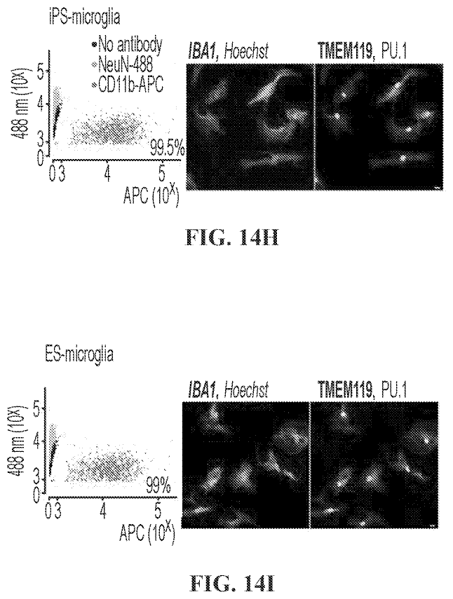

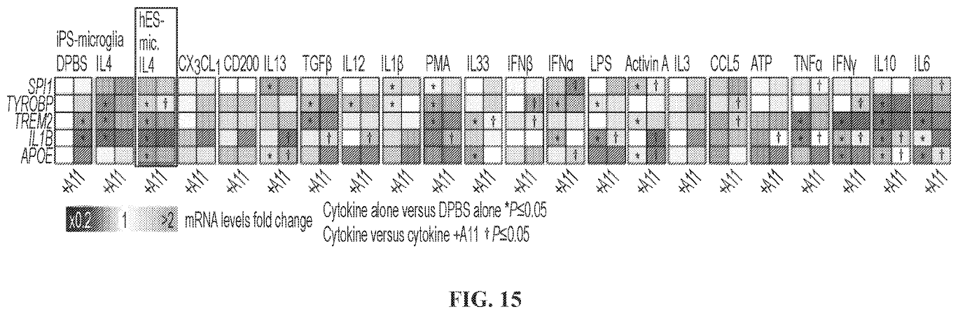

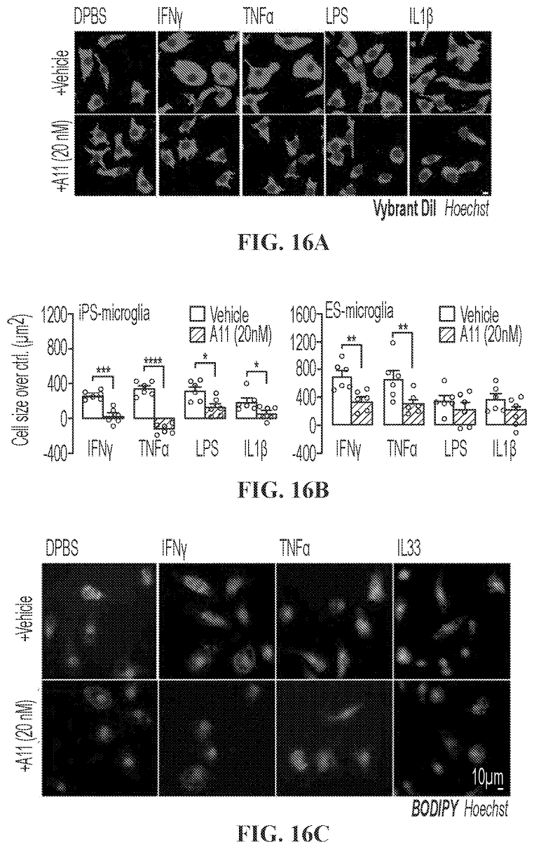

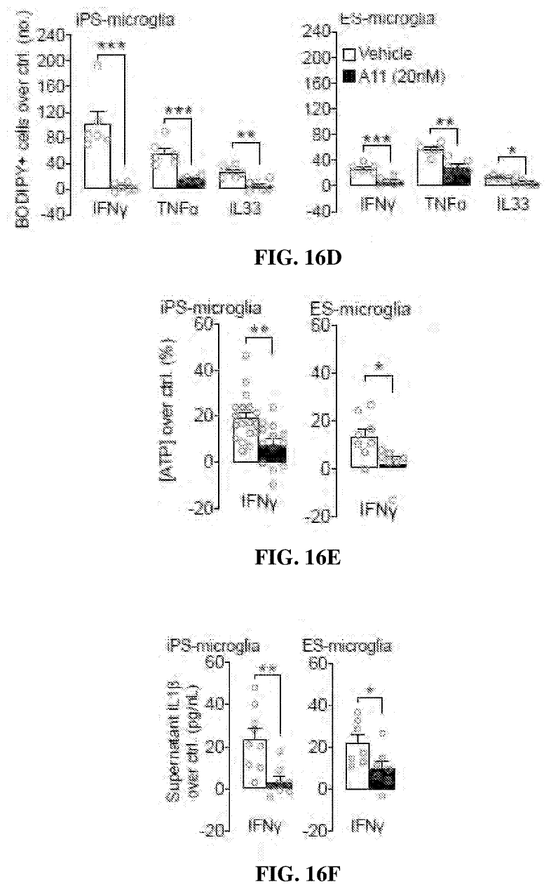

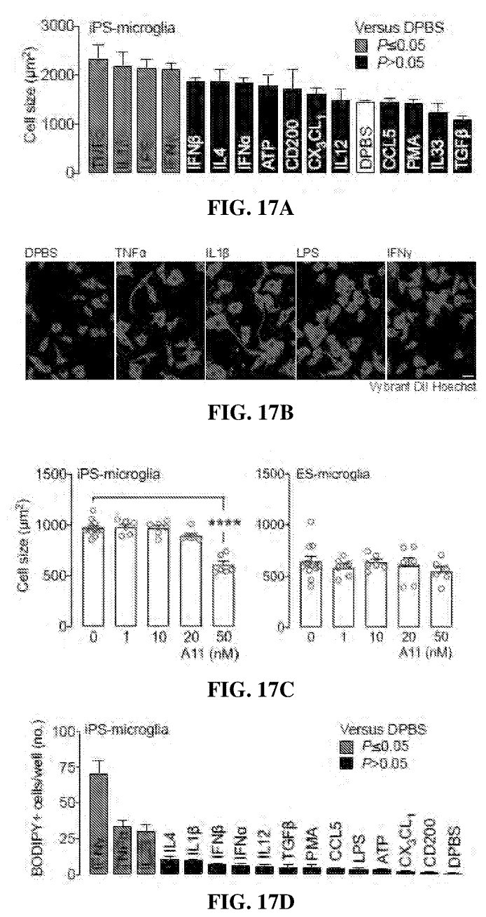

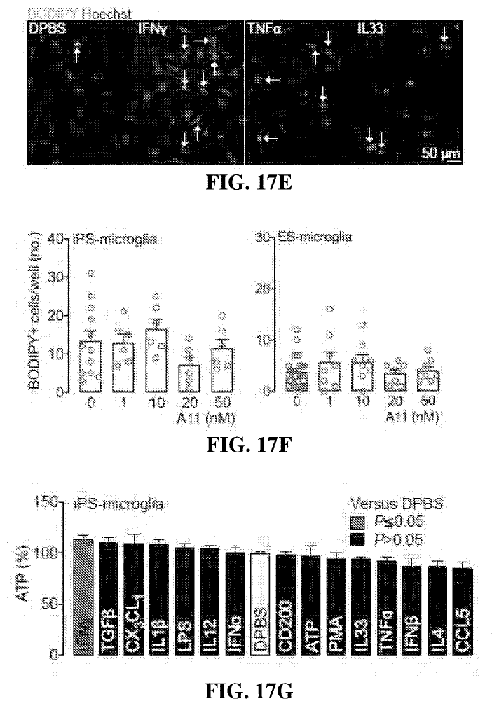

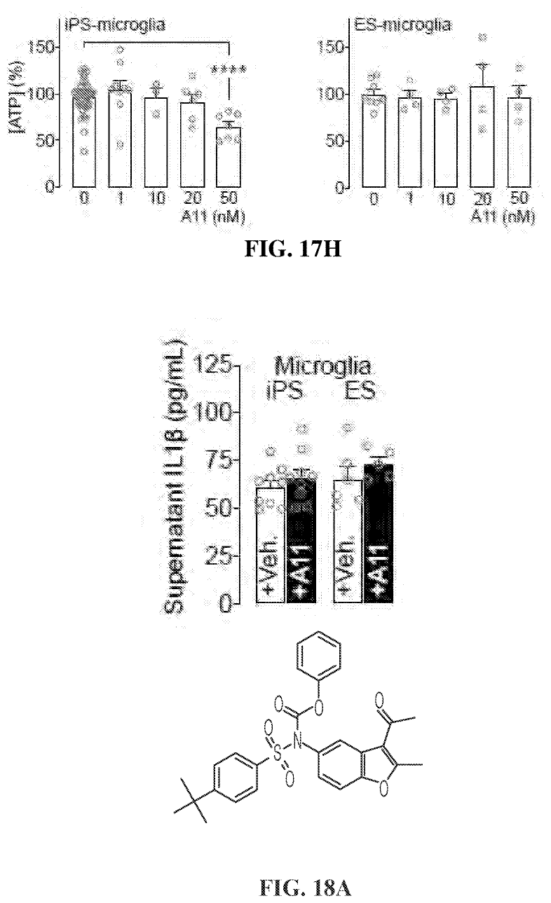

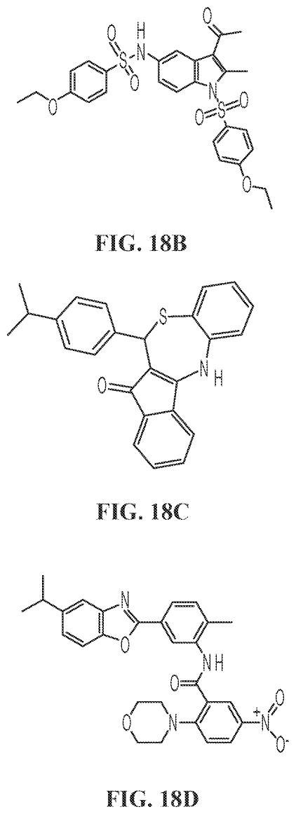

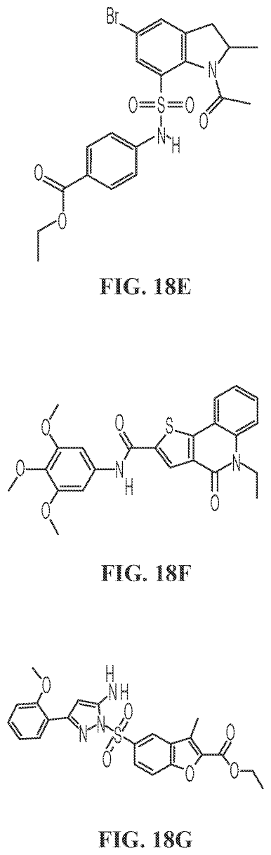

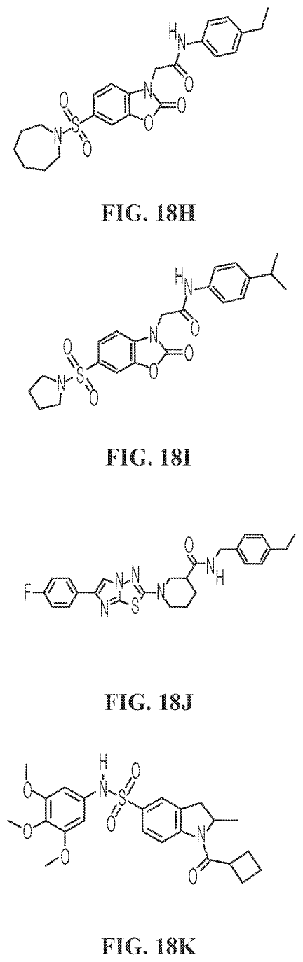

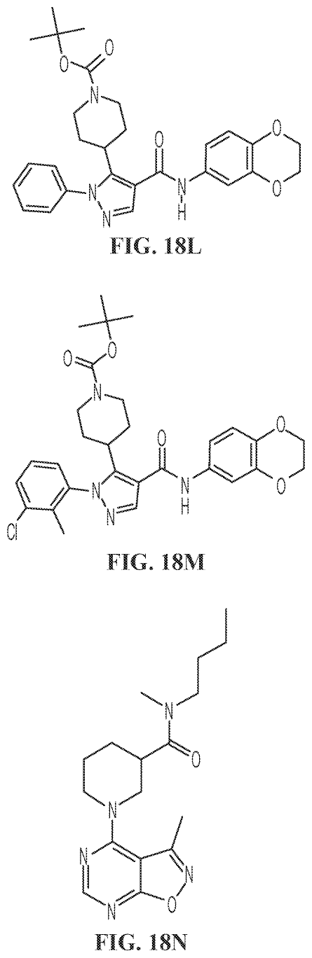

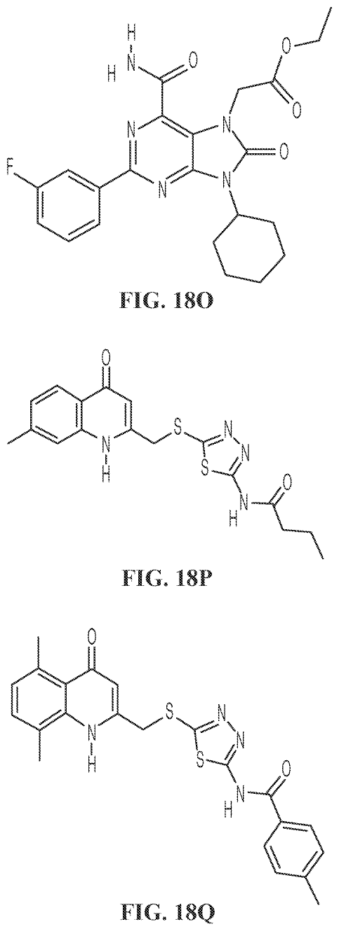

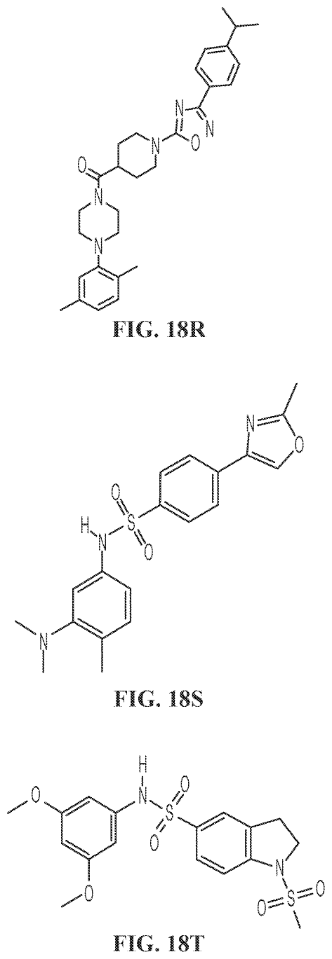

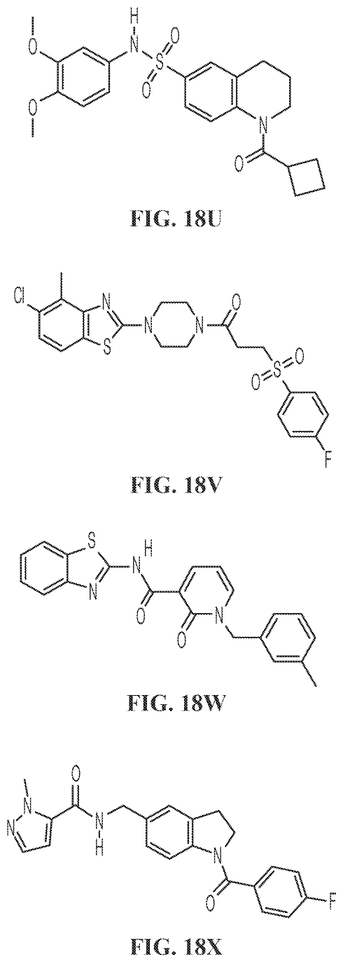

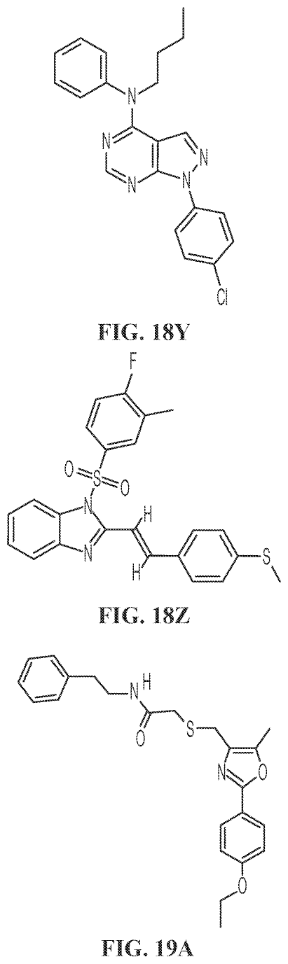

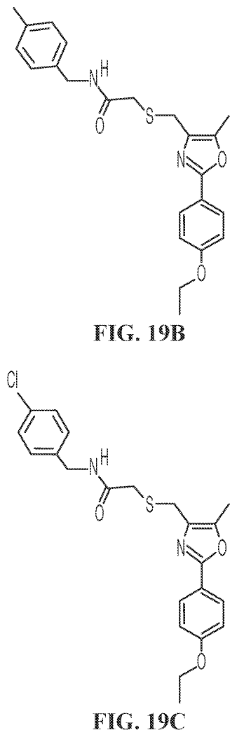

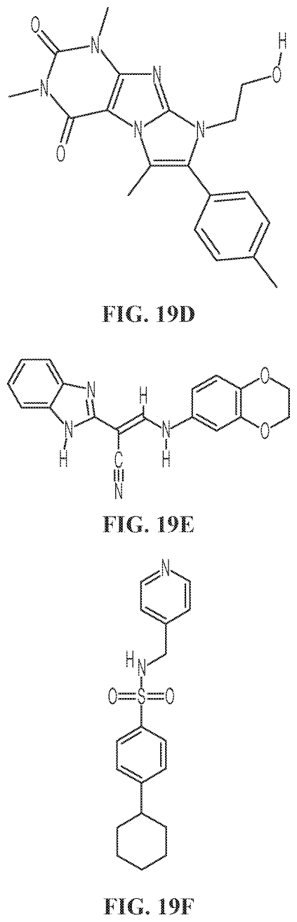

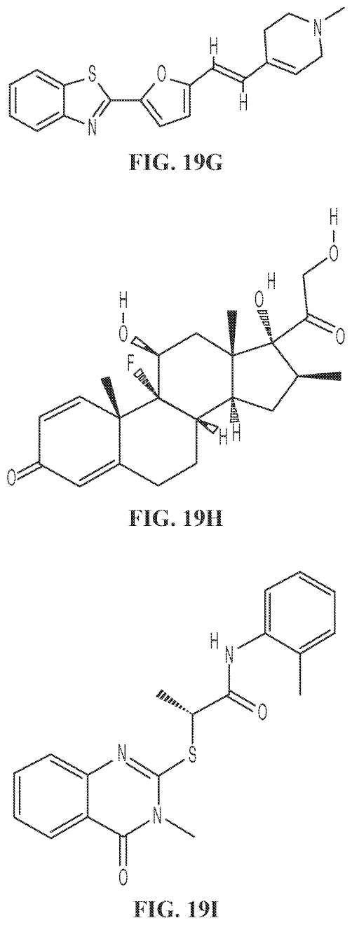

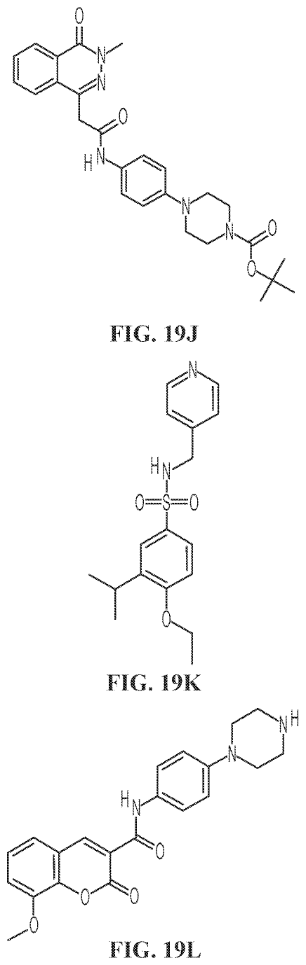

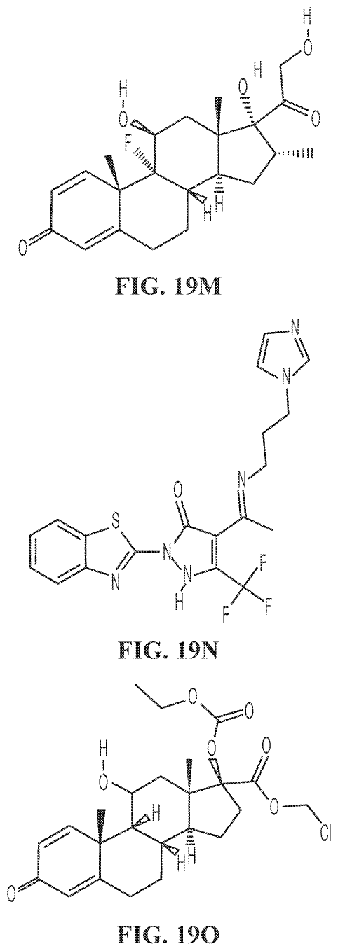

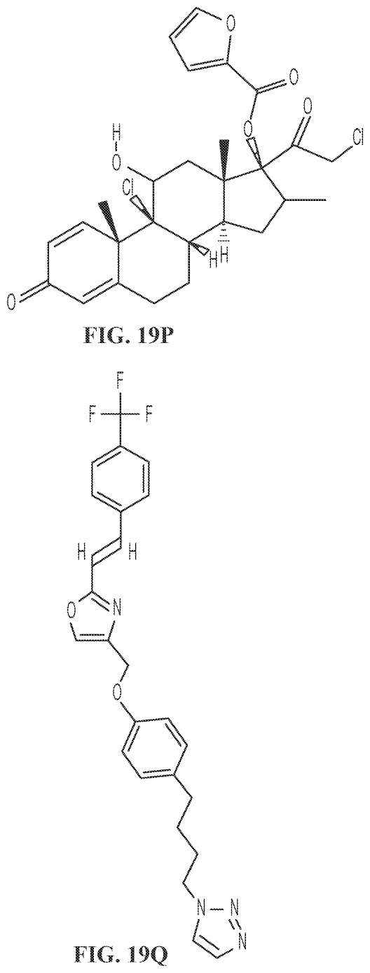

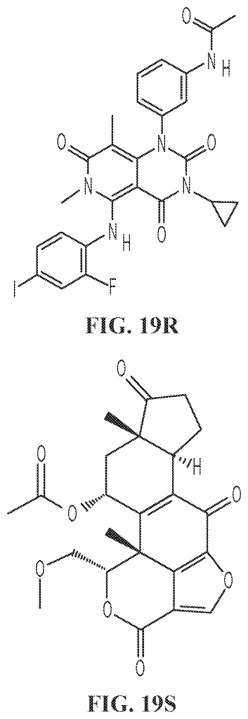

The BV2 PU.1-Luciferase cells (referred to as “cells” from here onward) were plated into a 384-well plate, using an automatic dispenser, 2000 cells per well in 30 μL of media composed of RPMI (Sigma-Aldrich), 10% FBS, 1% PenStrep (50 mg/mL) and 1% G418 (50 mg/mL) was plated. A robotic arm was used to transfer compounds in a 384-well format using a pin array into the plate containing the cells (here, 33 nL was transferred from a drug library containing 10 mM concentration of drugs for a final concentration of 10 μM per well). For reproducibility, each pin array with 384 compounds was added to 4 plates of cells. The cells and compounds were incubated at 37° C. for a period of time sufficient for the compounds to have an effect on the cells (about 2 days). Subsequently, automatic of 30 μL of BrightGlo (Promega) was dispensed into 2 of the 4 plates, in order to lyse the cells and drive the luminescence reaction whereafter the luminescence intensity was measured by a plate reader (Perkin Elmer EnVision). The two other plates were fixed by automatically dispensing 30 μL of 10% formaldehyde with 2μL Hoechst per 1 mL formaldehyde which were left at room temperature for one hour, followed by plate reading (Acumen (TTPLabTech)). A compound transfer and treatment would be repeated if reproducibility between the luminescence plates and/or the fixed Hoechst plates was lacking. The drugs were selected and ranked according to the criteria described in FIG. 13. Luminescence values used were corrected for crosstalk. Hoechst values were total fluorescence area per well. Determination of a positive hit considered both the reduction of luminescence as well as a possible confounding reduction in cell viability. The formula used was y=(value−meanvalue)/stddev. The formula classified compounds according to weak, medium, and strong luminescence reducers, such that weak luminescence reducers had 2.5<y<3 standard deviations reduction form the mean value of all wells, medium luminescence reducers had 3<y<4 standard deviations reduction, and strong reducers had >4 standard deviations difference form the mean. Compounds were excluded if cell viability was reduced >2 standard deviations from the mean, using the same formula. The positive hits were then validated in multiple steps in order to identify true PU.1 inhibitors.

### Screened Libraries

The IACS Selleck Known Bioactive library (2,100 compounds) was screened and there 35 hits were identified (˜1.7% hit rate), the Asinex1 library (12,000 compounds) was screened and 60 hits were identified (˜0.5% hit rate), and the ChemDiv6 library (44,000 compounds) was screened and 169 hits were identified (˜0.4% hit rate). The Selleck library contains drugs approved by the United States Food and Drug Administration (FDA) with known intracellular mechanisms of action which will give instant insight into pathways that block PU.1 activity. The Asinex1 and ChemDiv6 libraries contain molecules with unknown biological properties, but with low toxicity, high stability, and a broad structural diversity used to generate structure-activity-relationships (SAR) of the PU.1 inhibitors. SAR is used to design new drug libraries containing only molecules with similar structures as previous hits. In order to obtain the SAR, three rounds of validation were performed. In round 1, false-positive, non-specific luciferase quencher molecules were excluded using the commercially available HEK293 CMV-Luciferase line that constitutively expresses luciferase under the CMV promoter. In round 2, weak (non-significant) hits were eliminated according to degree of PU.1 inhibition in the absence of cell death. In round 3, drugs were selected that reduce PU.1-dependent gene expression with RT-qPCR.

### Results and Methodology

### Initial Validation

The BV2 PU.1-Luciferase and HEK293 CMV-Luciferase cell lines were first validated with a set of control experiments. Assays were conducted for percent luminescence and cell viability (Hoechst-staining) after a 2-day incubation under the following conditions. As negative controls pure DMSO and “mock” conditions (where nothing is added to the cells) were used. As expected, there was no change in either luminescence or cell viability with these negative controls. Then a toxic dose (10 μM) of a translational inhibitor, actinomycin D was tested, and it was found that it indeed reduced cell viability in both lines. On the other hand, a toxic dose of the translational inhibitor puromycin (10 μM) (to which the HEK293 CMV-Luciferase line is resistant) only reduced cell viability in the BV2 PU.1-luciferase cells. In order to confirm that non-specific luciferase quenchers can be identified, 119113, a known luciferase inhibitor was included, at 10 μM and it inhibited luminescence in both lines without causing toxicity. The ultimate confirmation of the design of the first-in-class published PU.1 inhibitor DB2313 (Antony-Debré et al., 2017; applied at 2.5 μM, provided to us by MDAnderson) was tested by confirming it was ineffective in the HEK293 CMV-Luciferase line while reducing luminescence in the BV2 PU.1-Luciferase line. Indeed, a reduction in luminescence was seen as well as cell viability in the BV2 PU.1-Luciferase line but no effect in the HEK293 CMV-Luciferase cells.

Results from the High-Throughput Drug Screen

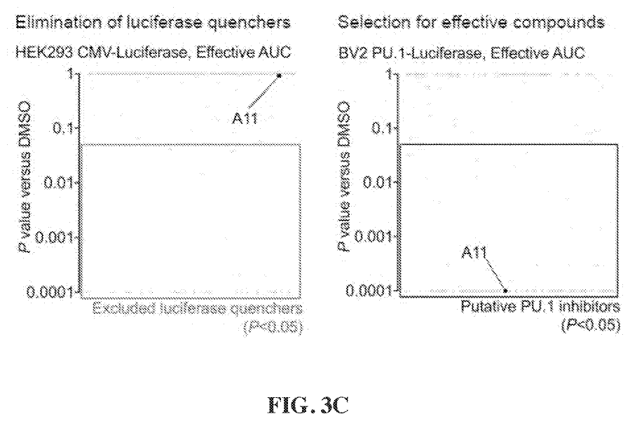

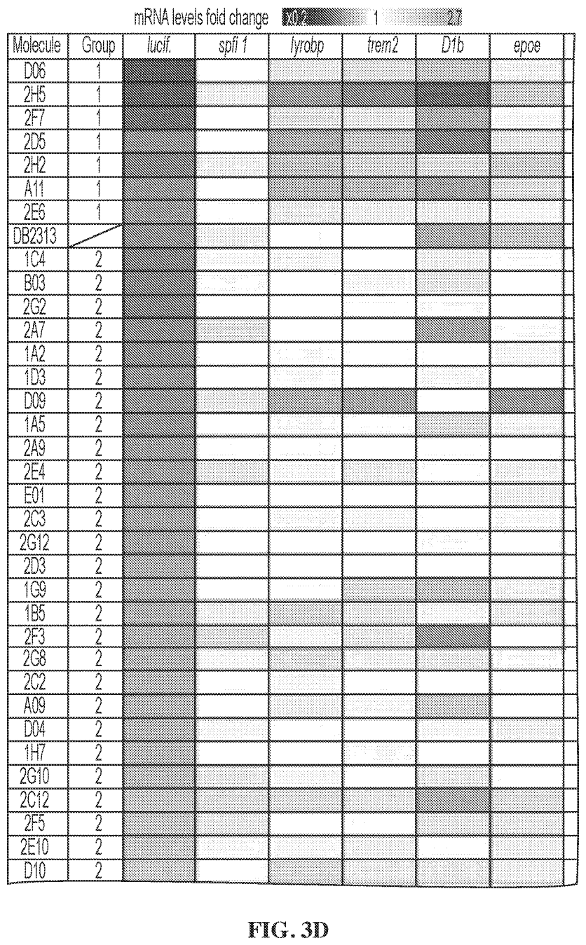

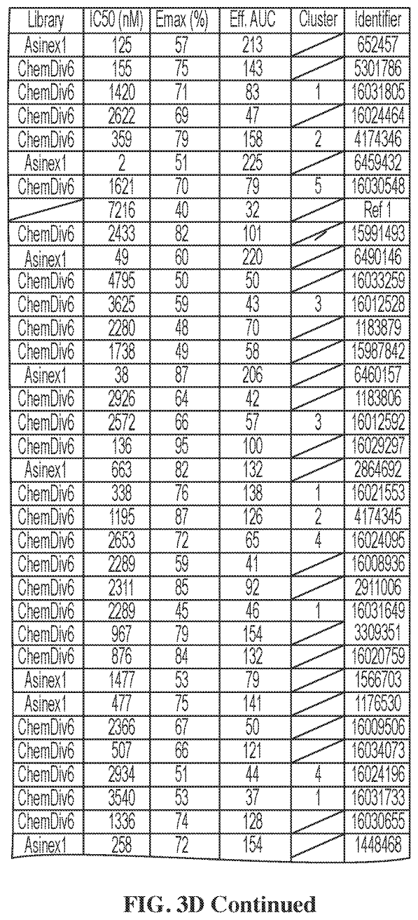

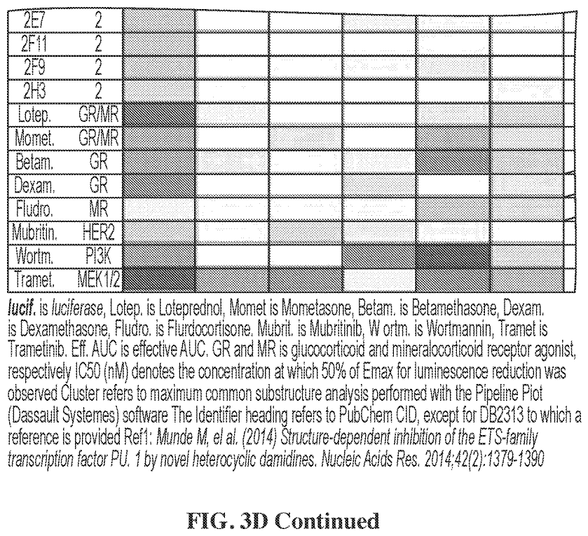

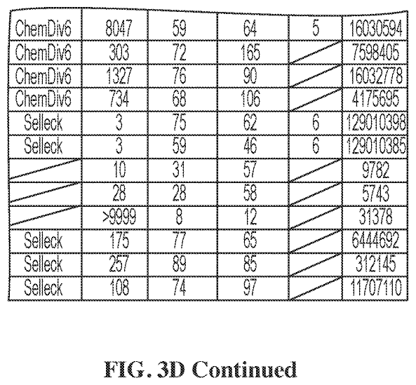

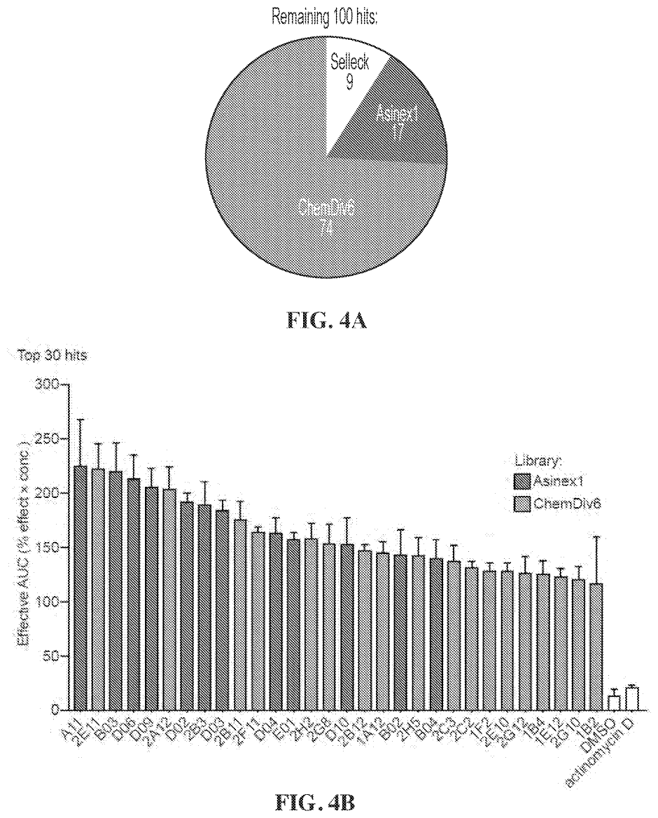

FIG. 5 illustrates the drug libraries screened and the final outcome after 3 steps of validation. In step 1, 64 non-specific luciferase inhibitors were eliminated by use of the HEK293 CMV-Luciferase cell line, leaving 200 out of 264 compounds (i.e., 24% were false positives) (see FIG. 3B). In step 2, weak or ineffective compounds were further eliminated by ranking according to PU.1 inhibition from 600 pM to 2.5 μM in the BV2 PU.1-Luciferase cell line (see FIG. 3A). The percent cell death was subtracted from the percent reduced luminescence over the entire concentration range, yielding an “effective area under the curve” (see FIGS. 3A-3B and 4A-4B). Only 86 out of the 200 remaining hits had a significant effective area under the curve versus vehicle. From the initial 58,100 compounds, this yields a hit rate of 0.15%. In the final and 3rd step, RT-qPCR was performed on the remaining 86 hits in BV2 PU.1-Luciferase cells in order to find the compounds that significantly reduced PU.1 dependent expression. In parallel, structure activity relationship of all 86 hits was analyzed in order to identify common functional groups.

From the RT-qPCR, there was a final hit rate of 40 compounds that significantly reduced luciferase expression (Table 1), used as a proxy for PU.1 dependent gene expression. These 40 hits were subdivided based on their effect on the other genes tested (Spil (PU.1 mRNA), Tyrobp, Trem2, IL1β, Apoe) into three groups. Group 1 (see Table 1) consists of hits that reduced not only luciferase expression, but also IL1β expression, in addition to increasing expression of Tyrobp, Trem2 and Apoe. The most potent (lowest IC50) is A11, marked with a box with a value of 2 nM. Group 2 (Table 1) include hits that only show reduced luciferase expression in common, for reference, the first-in-class PU.1 inhibitor DB2313 is included on top. Group 3 (Table 1) contains hits from the Selleck library—from these hits the drug class of glucocorticoid receptor (but not mineralocorticoid receptor) agonists was identified as PU.1 inhibitors.

### Structure Activity Relationship

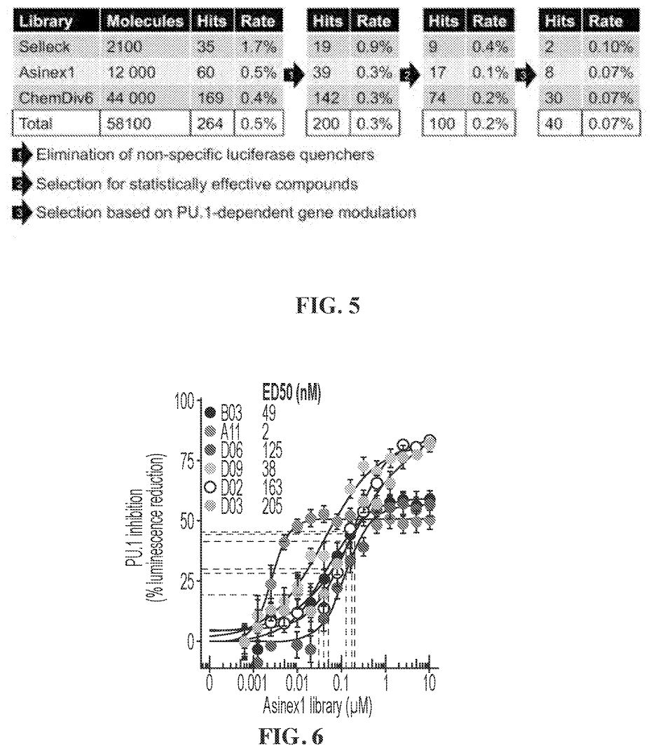

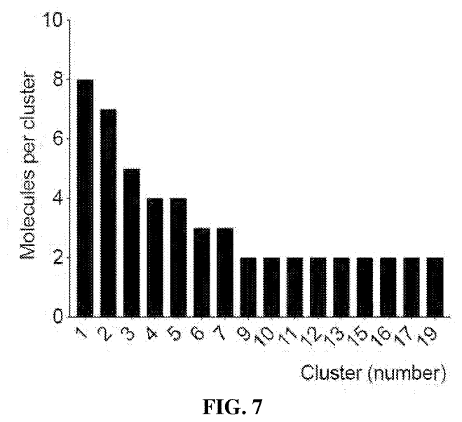

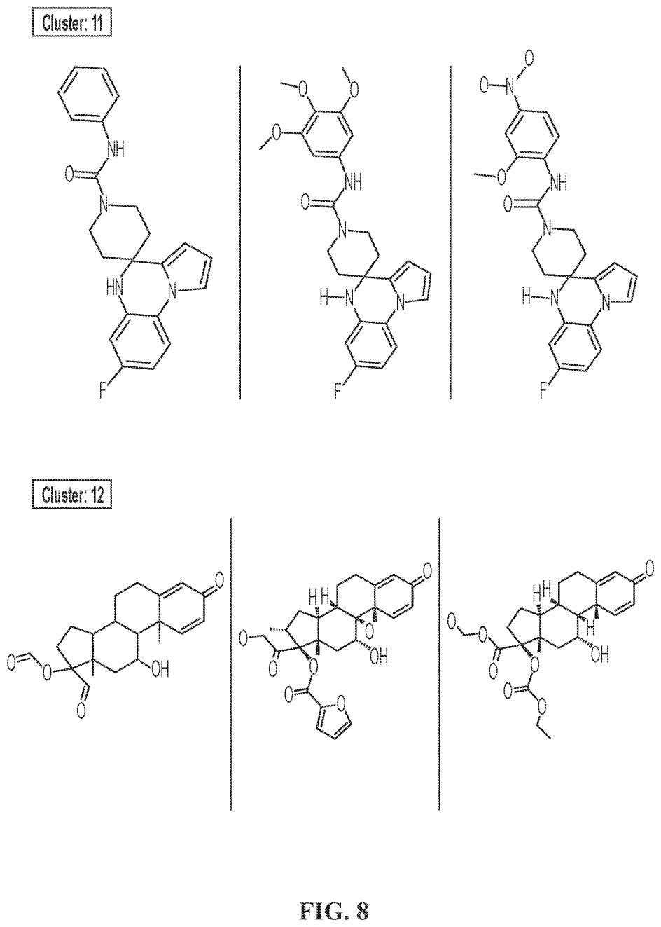

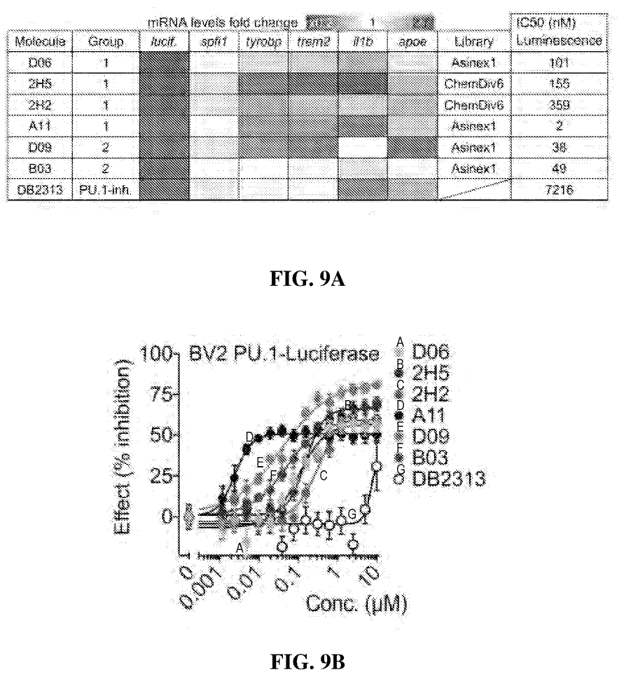

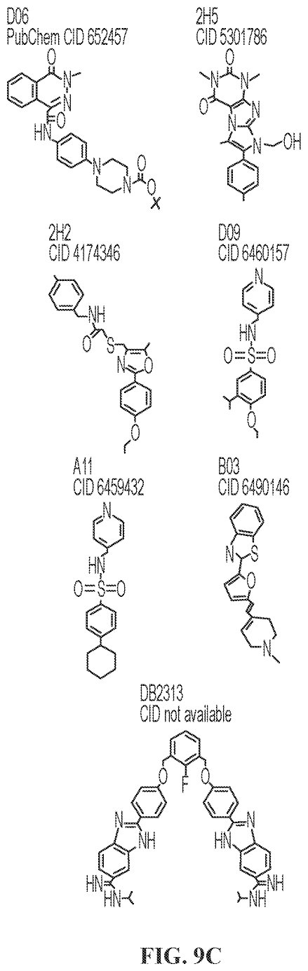

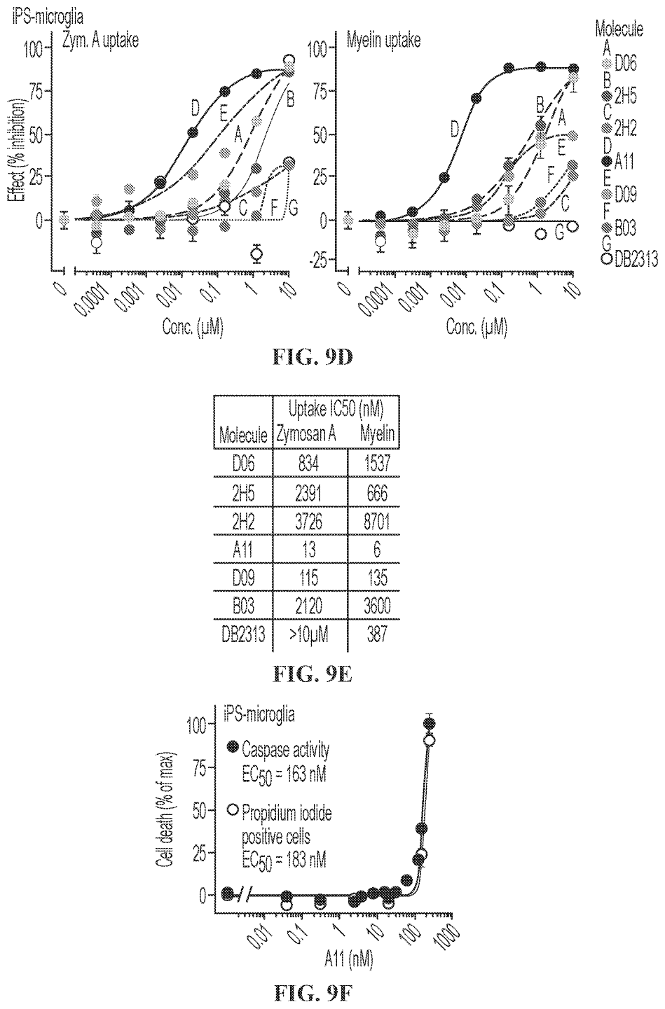

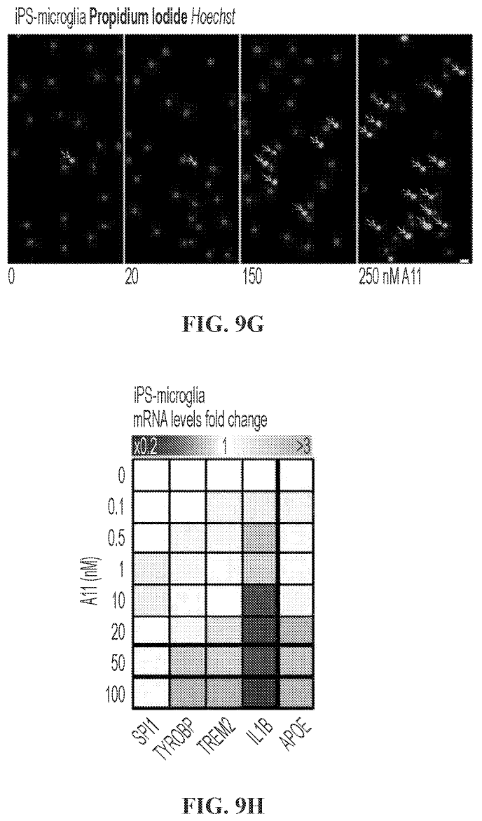

The degree of luminescence reduction of the hits was plotted with significant effective area under the curve in order to obtain an IC50. The top six hits from the Asinex1 library are shown in FIG. 6. A summary of the clusters and number of molecules per cluster is shown in FIG. 7 and two examples of clusters with corresponding hits in FIG. 8.

A11 has Anti-Inflammatory Properties in iPS-Derived Microglia

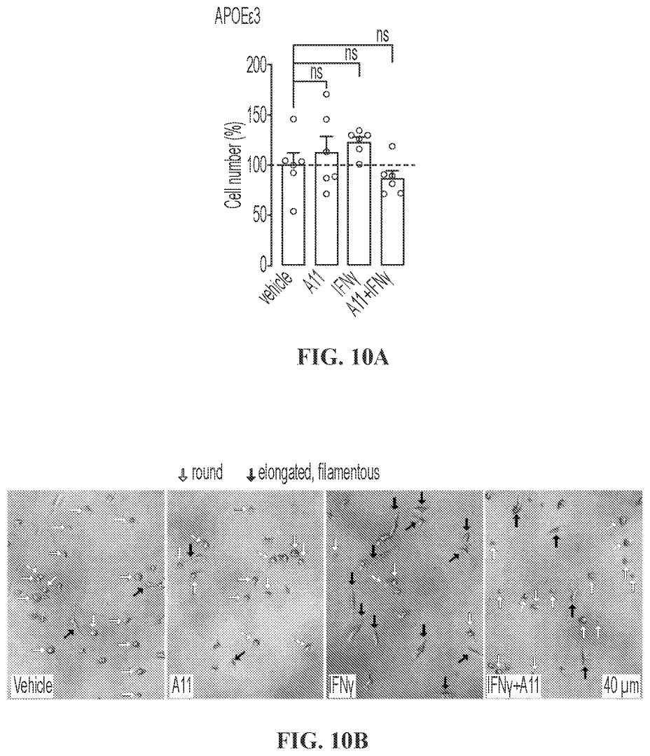

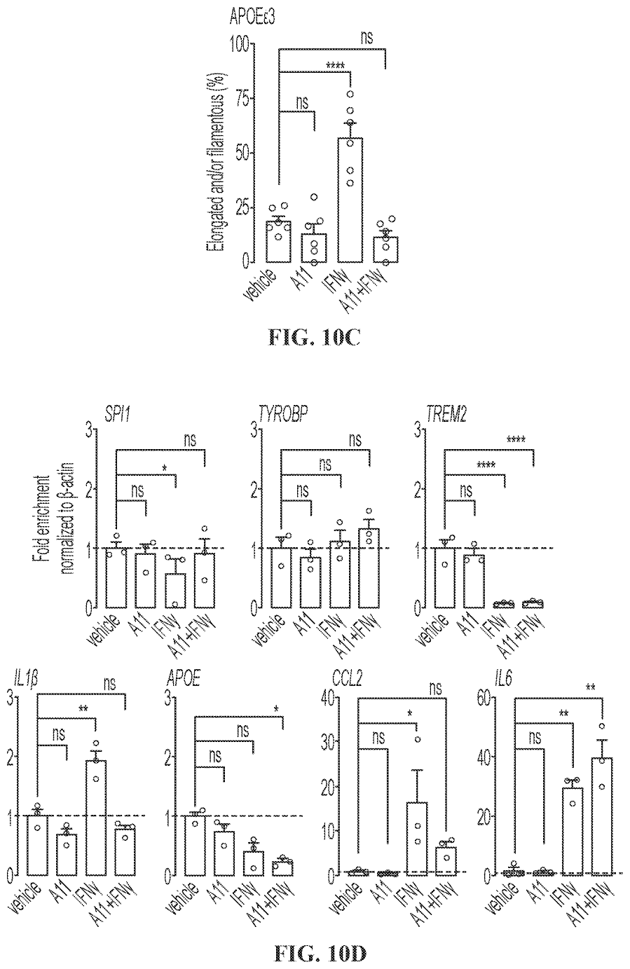

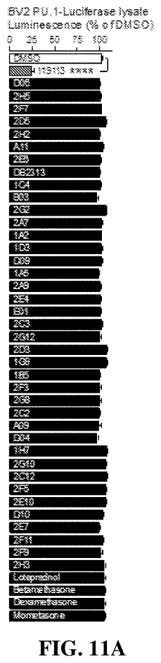

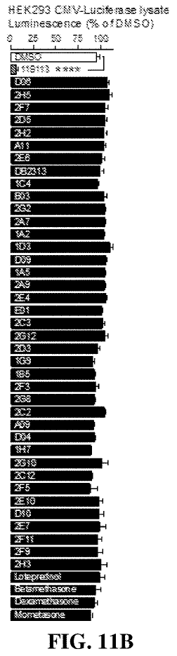

Human induced pluripotent stem cell derived microglia (iPSC-microglia) were activated with IFN-gamma (25 ng/mL, 3 days) and co-treated with A11 (2.5 μM, 2 days). No significant change was observed in cell viability (FIG. 10A), but a reversal of the elongated and filamentous morphology induced by IFN-gamma (FIG. 10B, quantified in FIG. 10C). Messenger RNA (mRNA) expression levels showed a reversal of IL1-beta and CCL2 increases to vehicle treated levels by A11 treatment.

### A11 Reduces Myelin Uptake

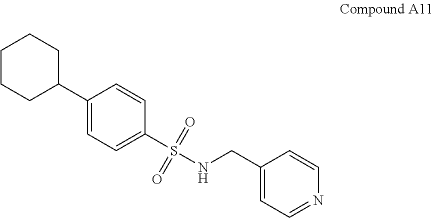 [SMILES: O=S(=O)(NCc1ccncc1)c1ccc(C2CCCCC2)cc1]

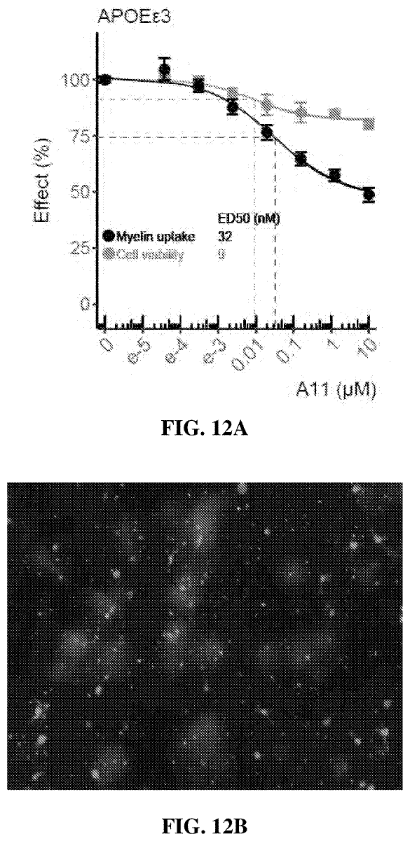

A concentration response curve was performed for cell viability and myelin uptake (FIG. 12A) in iPSC-microglia. Myelin uptake was measured by uptake of pHrodo-GFP-labelled wild-type mouse myelin particles (FIG. 12B). A weak reduction in cell viability and a maximal possible reduction in myelin uptake of 50% were found. The IC50s obtained for these assays was in the low nM range, matching the data obtained in BV2 cells.

TABLE 1
List of PU.1 Inhibitors
mRNA levels (represented as multiples of 1×)

| | | | | | | | | | IC50 | |
|---|---|---|---|---|---|---|---|---|---|---|
| Compound | Group | Lucif. | Spi1 | Tyrobp | Trem2 | IL1β | Apoe | Library | (nM) | Cluster |
| D06 | 1 | 0.153 | 1.030 | 1.374 | 1.510 | 0.590 | 1.233 | Asinexl | 25 | 19 |
| 2H5 | 1 | 0.165 | 1.316 | 2.422 | 2.628 | 0.185 | 1.756 | ChemDiv6 | 155 | — |
| 2F7 | 1 | 0.216 | 0.936 | 1.498 | 1.442 | 0.460 | 1.146 | ChemDiv6 | 1420 | 1 |
| 2D5 | 1 | 0.359 | 1.015 | 2.257 | 1.852 | 0.287 | 1.400 | ChemDiv6 | 2622 | — |
| 2H2 | 1 | 0.299 | 1.251 | 1.952 | 1.668 | 0.706 | 1.739 | ChemDiv6 | 380 | 2 |
| A11 | 1 | 0.376 | 1.001 | 2.101 | 2.051 | 0.418 | 1.522 | Asinex1 | 2 | 9 |
| 2E6 | 1 | 0.379 | 1.046 | 1.347 | 1.262 | 0.810 | 1.170 | ChemDiv6 | 1621 | 10 |
| DB2313 | PU.1-inh. | 0.340 | 0.820 | 1.030 | 1.030 | 0.460 | 0.600 | — | — | n.d. |
| 1C4 | 2 | 0.290 | 1.142 | 1.197 | 0.928 | 0.813 | 0.883 | ChemDiv6 | 2433 | 5 |
| B03 | 2 | 0.291 | 0.885 | 0.922 | 0.863 | 0.767 | 1.035 | Asinex1 | 49 | — |
| 2G2 | 2 | 0.294 | 0.958 | 0.963 | 1.071 | 0.887 | 0.957 | ChemDiv6 | 4795 | — |
| 2A7 | 2 | 0.328 | 0.739 | 0.928 | 0.942 | 0.435 | 0.886 | ChemDiv6 | 3625 | 4 |
| 1A2 | 2 | 0.324 | 0.915 | 1.093 | 1.073 | 1.054 | 0.858 | ChemDiv6 | 2280 | 16 |
| 1D3 | 2 | 0.347 | 0.965 | 1.102 | 1.031 | 1.144 | 0.928 | ChemDiv6 | 1738 | 1 |
| D09 | 2 | 0.360 | 0.781 | 0.496 | 0.437 | 0.938 | 0.390 | Asinex1 | 38 | 2 |
| 1A5 | 2 | 0.371 | 0.982 | 1.117 | 1.085 | 0.707 | 1.200 | ChemDiv6 | 2926 | — |
| 2A9 | 2 | 0.391 | 1.073 | 1.059 | 0.958 | 1.048 | 0.988 | ChemDiv6 | 2572 | 4 |
| 2E4 | 2 | 0.404 | 1.377 | 1.141 | 0.806 | 1.077 | 0.841 | ChemDiv6 | 136 | 13 |
| E01 | 2 | 0.416 | 0.912 | 0.988 | 0.903 | 0.986 | 0.823 | Asinex1 | 663 | 17 |
| 2C3 | 2 | 0.430 | 0.946 | 1.127 | 1.179 | 1.043 | 1.133 | ChemDiv6 | 338 | 1 |
| 2G12 | 2 | 0.437 | 1.065 | 0.994 | 1.024 | 1.097 | 1.036 | ChemDiv6 | 1195 | 2 |
| 2D3 | 2 | 0.480 | 0.922 | 0.950 | 0.987 | 0.960 | 0.980 | ChemDiv6 | 2653 | 6 |
| 1G9 | 2 | 0.485 | 0.989 | 1.053 | 0.713 | 0.582 | 1.217 | ChemDiv6 | 2289 | — |
| 1B5 | 2 | 0.491 | 0.831 | 0.697 | 0.775 | 0.843 | 0.797 | ChemDiv6 | 2311 | — |
| 2F3 | 2 | 0.496 | 0.733 | 0.854 | 0.790 | 0.386 | 1.011 | ChemDiv6 | 2289 | 1 |
| 2G8 | 2 | 0.497 | 1.179 | 1.469 | 1.237 | 1.362 | 1.119 | ChemDiv6 | 967 | — |
| 2C2 | 2 | 0.504 | 1.034 | 1.130 | 1.031 | 0.954 | 1.035 | ChemDiv6 | 876 | 3 |
| A09 | 2 | 0.513 | 0.931 | 0.821 | 0.879 | 0.644 | 0.941 | Asinex1 | 1477 | — |
| D04 | 2 | 0.519 | 0.865 | 1.071 | 0.976 | 0.866 | 0.841 | Asinex1 | 477 | 19 |
| 1H7 | 2 | 0.524 | 1.022 | 1.011 | 1.100 | 1.017 | 0.908 | ChemDiv6 | 2366 | — |
| 2G10 | 2 | 0.531 | 0.790 | 0.855 | 0.993 | 0.786 | 0.899 | ChemDiv6 | 507 | 7 |
| 2C12 | 2 | 0.597 | 0.736 | 0.713 | 0.752 | 0.486 | 0.731 | ChemDiv6 | 2934 | 6 |
| 2F5 | 2 | 0.590 | 0.783 | 0.985 | 0.901 | 0.767 | 0.790 | ChemDiv6 | 3540 | 1 |
| 2E10 | 2 | 0.611 | 1.133 | 1.158 | 1.253 | 1.082 | 1.244 | ChemDiv6 | 1336 | — |
| D10 | 2 | 0.622 | 1.072 | 1.593 | 1.342 | 1.347 | 1.251 | Asinex1 | 258 | — |
| 2E7 | 2 | 0.623 | 0.926 | 0.927 | 0.854 | 0.887 | 1.076 | ChemDiv6 | 8047 | 10 |
| 2F11 | 2 | 0.640 | 0.943 | 1.002 | 1.012 | 1.086 | 1.005 | ChemDiv6 | 303 | — |
| 2F9 | 2 | 0.655 | 1.020 | 0.904 | 0.890 | 0.821 | 0.928 | ChemDiv6 | 1327 | — |
| 2H3 | 2 | 0.722 | 0.897 | 1.003 | 0.986 | 0.915 | 1.111 | ChemDiv6 | 734 | 2 |
| Loteprednol | GR/MR | 0.265 | 1.167 | 0.927 | 0.979 | 0.805 | 1.579 | Selleck | 3 | 12 |
| Mometasone | GR/MR | 0.520 | 1.016 | 1.343 | 1.051 | 0.531 | 1.444 | Selleck | 3 | 12 |
| Betamethasone | GR | 0.481 | 1.235 | 1.079 | 1.137 | 0.379 | 1.615 | — | 10 | n.d. |
| Dexamethasone | GR | 0.373 | 0.955 | 1.010 | 0.751 | 1.041 | 1.325 | — | 28 | n.d. |
| Fludrocortisone | MR | 1.171 | 0.900 | 1.082 | 0.895 | 1.764 | 1.493 | — | — | n.d. |

An example of Selection and Ranking of PU.1 using the assays described herein and presented in FIG. 13 was presented in Table 2 of US provisional application 62/900328, filed Sep. 13, 2019, to which the instant application claims priority and which is incorporated by reference in its entirety including the entirety of Table 2 disclosed therein.

### REFERENCES

1. Abud E M, Ramirez R N, Martinez E S, Healy L M, Nguyen C H H, Newman S A, Yeromin A V, Scarfone V M, Marsh S E, Fimbres C, Caraway C A, Fote G M, Madany A M, Agrawal A, Kayed R, Gylys K H, Cahalan M D, Cummings B J, Antel J P, Mortazavi A, Carson M J, Poon W W, Blurton-Jones M. (2017). iPSC-Derived Human Microglia-like Cells to Study Neurological Diseases. Neuron. 94(2): 278-293.e9.

2. Cong L, Ran F A, Cox D, Lin S, Barretto R, Habib N, Hsu P D, Wu X, Jiang W, Marraffini L A, Zhang F. (2013). Multiplex genome engineering using CRISPR/Cas systems. Science. 339(6121): 819-23.

3. Dejanovic B, Huntley M A, De Mazière A, Meilandt W J, Wu T, Srinivasan K, Jiang Z, Gandham V, Friedman B A, Ngu H, Foreman O, Carano R A D, Chih B, Klumperman J, Bakalarski C, Hanson J E, Sheng M. (2018). Changes in the Synaptic Proteome in Tauopathy and Rescue of Tau-Induced Synapse Loss by C1q Antibodies. Neuron. 2018 October 30. pii: S0896-6273(18)30902-4.

4. Gilbert L A, Larson M H, Morsut L, Liu Z, Brar G A, Torres S E, Stern-Ginossar N, Brandman O, Whitehead E H, Doudna J A, Lim W A, Weissman J S, Qi LS. (2013). CRISPR-mediated modular RNA-guided regulation of transcription in eukaryotes. Cell. 154(2): 442-51.

5. Guerreiro R, Wojtas A, Bras J, Carrasquillo M, Rogaeva E, Majounie E, Cruchaga C, Sassi C, Kauwe J S, Younkin S, Hazrati L, Collinge J, Pocock J, Lashley T, Williams J, Lambert J C, Amouyel P, Goate A, Rademakers R, Morgan K, Powell J, St George-Hyslop P, Singleton A, Hardy J; Alzheimer Genetic Analysis Group. (2013). TREM2 Variants in Alzheimer's Disease. N Engl J Med. 368(2): 117-127.

6. Hong S, Beja-Glasser V F, Nfonoyim B M, Frouin A, Li S, Ramakrishnan S, Merry K M, Shi Q, Rosenthal A, Barres B A, Lemere C A, Selkoe D J, Stevens B. (2016) Complement and microglia mediate early synapse loss in Alzheimer mouse models. Science. 352(6286): 712-716.

7. Jonsson T, Stefansson H, Steinberg S, Jonsdottir I, Jonsson PV, Snaedal J, Bjornsson S, Huttenlocher J, Levey AI, Lah J J, Rujescu D, Hampel H, Giegling I, Andreassen O A, Engedal K, Ulstein I, Djurovic S, Ibrahim-Verbaas C, Hofman A, Ikram MA, van Duijn C M, Thorsteinsdottir U, Kong A, Stefansson K. (2013). Variant of TREM2 Associated with the Risk of Alzheimer's Disease. N Engl J Med. 368(2): 107-116.

8. Maeder M L, Linder S J, Cascio V M, Fu Y, Ho Q H, Joung J K. (2013). CRISPR RNA-guided activation of endogenous human genes. Nat Methods. 10(10): 977-9.

9. Muffat J, Li Y, Yuan B, Mitalipova M, Omer A, Corcoran S, Bakiasi G, et al., (2016). Efficient derivation of microglia-like cells from human pluripotent stem cells. Nat Med 22(11): 1358-1367.

10. Penney J, Ralvenius W R, Tsai L H (2019). Modeling Alzheimer's disease with iPSC-derived brain cells. Molecular Psychiatry. In Press.

11. Song W M, Joshita S, Zhou Y, Ulland T K, Gilfillan S, Colonna M. (2018). Humanized TREM2 mice reveal microglia-intrinsic and -extrinsic effects of R47H polymorphism. J Exp Med. 215(3): 745-760.

12. Ulrich J D, Ulland T K, Colonna M, Holtzman D M. (2017). Elucidating the Role of TREM2 in Alzheimer's Disease. Neuron. 94(2): 237-248.

13. Cataldo, A., Peterhoff, C., Troncoso, J., Gomez-Isla, T., Hyman, B., & Nixon, R. (2000). Endocytic Pathway Abnormalities Precede Amyloid β Deposition in Sporadic Alzheimer's Disease and Down Syndrome. The American Journal Of Pathology, 157(1), 277-286.

14. Cataldo, A., Rebeck, G., Ghetti, B., Hulette, C., Lippa, C., & Van Broeckhoven, C. et al. (2001). Endocytic disturbances distinguish among subtypes of alzheimer's disease and related disorders. Annals Of Neurology, 50(5), 661.

15. Fu Y, Hsiao J H, Paxinos G, Halliday G M, Kim W S. ABCA7 Mediates Phagocytic Clearance of Amyloid-β in the Brain. J Alzheimers disease. 54(2), 569-84, 2016.

16. Hollingworth, P., Harold, D., Sims, R., Gerrish, A., Lambert, J. C., Carrasquillo, M. M. et al., (2011). Common variants at ABCA7, MS4A6A/MS4A4E, EPHA1, CD33 and CD2AP are associated with Alzheimer's disease. Nature Genetics, 43(5): 429-35.

17. Kim, W. S.; Fitzgerald, M. L.; Kang, K. W.; Okuhira, K.; Bell, S. A.; Manning, J. J.; Koehn, S. L.; Lu, N. F.; Moore, K. J.; Freeman, M. W. ABCA7 null mice retain normal macrophage phosphatidyleholine and cholesterol efflux activity despite alterations in adipose mass and serum cholesterol levels. J. Biol. Chem. 2005, 280, 3989-3995.

18. Kim, D., Frank, C., Dobbin, M., Tsunemoto, R., Tu, W., & Peng, P. et al. (2008). Deregulation of HDAC1 by p25/Cdk5 in Neurotoxicity. Neuron, 60(5), 803-817.

19. Kim W S, Li H, Ruberu K, Chan S, Elliott D A, Low J K, Cheng D, Karl T, Garner B. Deletion of Abca7 increases cerebral amyloid-β accumulation in the J20 mouse model of Alzheimer's disease. (2013). J Neuroscience 33(10), 4387-94.

20. Loregger A., Nelson J. K., Zelcer N. (2017) Assaying Low-Density-Lipoprotein (LDL) Uptake into Cells. In: Gelissen I., Brown A. (eds) Cholesterol Homeostasis. Methods in Molecular Biology, vol 1583. Humana Press, New York, N.Y.

21. Sakae, N.; Liu, C. C.; Shinohara, M.; Frisch-Daiello, J.; Ma, L.; Yamazaki, Y.; Tachibana, M.; Younkin, L.; Kurti, A.; Carrasquillo, M. M.; et al. ABCA7 deficiency accelerates amyloid-beta generation and alzheimer's neuronal pathology. J. Neurosci. 2016, 36, 3848-3859.

22. Steinberg S., Stefansson, H., Jonsson, T., Johannsdottir, H., Ingason, A., & Helgason, H. et al. (2015). Loss-of-function variants in ABCA7 confer risk of Alzheimer's disease. Nature Genetics, 47(5), 445-447.

23. Vasquez, J. B., Fardo, D. W., Estus S., (2013) ABCA7 expression is associated with Alzheimer's disease polymorphism and disease status. Neuroscience Letters, 556: 58-62

24. Choi S H, Kim Y H, Quinti L, Tanzi R E, Kim D Y. (2014). A three-dimensional human neural cell culture model of Alzheimer's disease. Nature. November 13; 515(7526): 274-8.

25. Qian X, Nguyen H N, Song M M, Hadiono C, Ogden S C, Hammack C, Yao B, Hamersky G R, Jacob F, Zhong C, Yoon K J, Jeang W, Lin L, Li Y, Thakor J, Berg D A, Zhang C, Kang E, Chickering M, Nauen D, Ho C Y, Wen Z, Christian K M, Shi P Y, Maher B J, Wu H, Jin P, Tang H, Song H, Ming G L. (2016). Brain-region-specific organoids using mini-bioreactors for modeling ZIKV exposure. Cell. May 19; 165(5): 1238-1254.

26. Raja W K, Mungenast A E, Lin Y T, Ko T, Abdurrob F, Seo J, Tsai L H. (2016) Self-organizing 3D human neural tissue derived from Induced pluripotent stem cells recapitulate Alzheimer's disease phenotypes. PLoS One. 2016 Sep. 13; 11(9).

27. Wang, Y and Mandelkow E (2016). Tau in physiology and pathology. Nat Rev Neurosci. January; 17(1): 5-21. Wolfe, M S (2009). Tau mutations in neurodegenerative diseases. J Biol Chem. March 6; 284(10): 6021-5.

28. Lin, Y.-T. et al. APOE4 Causes Widespread Molecular and Cellular Alterations Associated with Alzheimer's Disease Phenotypes in Human iPSC-Derived Brain Cell Types. Neuron 98, 1141-1154.e7 (2018).

29. Johnson L A, Torres E R, Impey S, Stevens J F, Raber J. Apolipoprotein E4 and Insulin Resistance Interact to Impair Cognition and Alter the Epigenome and Metabolome. Sci Rep. 2017 Mar. 8; 7: 43701.

30. Nuriel T, Peng K Y, Ashok A, Dillman A A, Figueroa H Y, Apuzzo J, Ambat J, Levy E, Cookson M R, Mathews P M, Duff K E. The Endosomal-Lysosomal Pathway Is Dysregulated by APOE4 Expression in vivo. Front Neurosci. 2017 Dec. 12; 11: 702.

31. Mann, K. M. et al. Independent effects of APOE on cholesterol metabolism and brain AP levels in an Alzheimer disease mouse model. Hum. Mol. Genet. 13, 1959-1968 (2004).

32. Mooijaart, S. P. et al. ApoE Plasma Levels and Risk of Cardiovascular Mortality in Old Age. PLOS Med. 3, e176 (2006).

33. Bales, K. R. et al. Apolipoprotein E is essential for amyloid deposition in the APPV717F transgenic mouse model of Alzheimer's disease. Proc. Natl. Acad. Sci. 96, 15233-15238 (1999).

34. Holtzman, D. M. et al. Expression of human apolipoprotein E reduces amyloid-β deposition in a mouse model of Alzheimer's disease. J. Clin. Invest. 103, R15-R21 (1999).

35. Koistinaho, M. et al. Apolipoprotein E promotes astrocyte colocalization and degradation of deposited amyloid-β peptides. Nat. Med. 10, 719-726 (2004).

36. Liao, F. et al. Murine versus human apolipoprotein E4: differential facilitation of and co-localization in cerebral amyloid angiopathy and amyloid plaques in APP transgenic mouse models. Acta Neuropathol. Commun. 3, 70 (2015).

37. Ulrich, J. D. & Holtzman, D. M. TREM2 Function in Alzheimer's Disease and Neurodegeneration. ACS Chem. Neurosci. 7, 420-427 (2016).

38. Yeh, F. L., Wang, Y., Tom, I., Gonzalez, L. C. & Sheng, M. TREM2 Binds to Apolipoproteins, Including APOE and CLU/APOJ, and Thereby Facilitates Uptake of Amyloid-Beta by Microglia. Neuron 91, 328-340 (2016).

39. Nitsch, R. M. et al. Evidence for a membrane defect in Alzheimer disease brain. Proc. Natl. Acad. Sci. 89, 1671-1675 (1992).

40. Ohm TG. The dentate gyrus in Alzheimer's disease. Prog Brain Res. 2007; 163: 723-40.

41. Yang C P, Gilley J A, Zhang G, Kernie S G. ApoE is required for maintenance of the dentate gyrus neural progenitor pool. Development. 2011 October; 138(20): 4351-62.

42. Leung L, Andrews-Zwilling Y, Yoon S Y, Jain S, Ring K, Dai J, Wang M M, Tong L, Walker D, Huang Y. Apolipoprotein E4 causes age-and sex-dependent impairments of hilar GABAergic interneurons and learning and memory deficits in mice. PLoS One. 2012; 7(12): e53569.

43. Marion-Poll L, Montalban E, Munier A, Herve D, Girault J A. Fluorescence-activated sorting of fixed nuclei: a general method for studying nuclei from specific cell populations that preserves post-translational modifications. Eur J Neurosci. 2014 April; 39(7): 1234-44.

44. Petersen, R. C. et al. Vitamin E and Donepezil for the Treatment of Mild Cognitive Impairment. N. Engl. J. Med. 352,2379-2388 (2005).

45. Wang, L. et al. The effect of APOE Ε4 allele on cholinesterase inhibitors in patients with Alzheimer's disease: Evaluation of the feasibility of resting state functional connectivity magnetic resonance imaging. Alzheimer Dis. Assoc. Disord. 28,122-127 (2014).

46. Hamilton, L. K. et al. Aberrant Lipid Metabolism in the Forebrain Niche Suppresses Adult Neural Stem Cell Proliferation in an Animal Model of Alzheimer's Disease. Cell Stem Cell 17, 397-411 (2015).

47. Ringman, J. M. et al. Clinical Predictors of Severe Cerebral Amyloid Angiopathy and Influence of APOE Genotype in Persons With Pathologically Verified Alzheimer Disease. JAMA Neural 71, 878-883 (2014).

48. Montagne A, Barnes S R, Sweeney M D, Halliday M R, Sagare A P, Zhao Z, Toga A W, Jacobs R E, Liu C Y, Amezcua L, Harrington M G, Chui H C, Law M, Zlokovic B V. Blood-brain barrier breakdown in the aging human hippocampus. Neuron. 2015 Jan. 21; 85(2): 296-302.

49. Greenberg, S. M., Rebeck, G. W., Vonsattel, J. P. G., Isla, T. G. & Hyman, B. T. Apolipoprotein E ϵ4 and cerebral hemorrhage associated with amyloid angiopathy. Ann. Neurol. 38, 254-259 (1995).

50. Premkumar, D. R., Cohen, D. L., Hedera, P., Friedland, R. P. & Kalaria, R. N. Apolipoprotein E-epsilon4 alleles in cerebral amyloid angiopathy and cerebrovascular pathology associated with Alzheimer's disease. Am. J. Pathol. 148, 2083 (1996).

51. Shinohara, M. et al. Impact of sex and APOE4 on cerebral amyloid angiopathy in Alzheimer's disease. Acta Neuropathol. 132, 225-234 (2016).

52. Jansen, W. J. et al. Prevalence of Cerebral Amyloid Pathology in Persons Without Dementia: A Meta-analysis. JAMA 313, 1924-1938 (2015).

53. Hollingworth, P. et al. Common variants at ABCA7, MS4A6A/MS4A4E, EPHA1, CD33 and CD2AP are associated with Alzheimer's disease. Nature Genetics 43, 429-435 (2011).

54. Lambert, J.-C. et al. Genome-wide association study identifies variants at CLU and CR1 associated with Alzheimer's disease. Nature Genetics 41, 1094-1099 (2009).

55. Lambert, J.-C. et al. Meta-analysis of 74,046 individuals identifies 11 new susceptibility loci for Alzheimer's disease. Nature Genetics 45,1452-1458 (2013).

56. Greenberg, S. M., Rebeck, G. W., Vonsattel, J. P. G., Isla, T. G. & Hyman, B. T. Apolipoprotein E ϵ4 and cerebral hemorrhage associated with amyloid angiopathy. Ann. Neurol. 38, 254-259 (1995).

57. Biffi, A. & Greenberg, S. M. Cerebral amyloid angiopathy: a systematic review. J Clin Neurol 7, 1-9 (2011).

58. Maloney, B., Ge, Y.-W., Alley, G. M. & Lahiri, D. K. Important differences between human and mouse APOE gene promoters: limitation of mouse APOE model in studying Alzheimer's disease. Journal of Neurochemistry 103, 1237-1257 (2007).

59. Reese, L. C. & Taglialatela, G. A role for calcineurin in Alzheimer's disease. Curr Neuropharmacol 9, 685-692 (2011).

60. Gwack, Y. et al. A genome-wide Drosophila RNAi screen identifies DYRK-family kinases as regulators of NFAT. Nature 441, 646-650 (2006).

61. Kapturczak, M. H., Meier-Kriesche, H. U. & Kaplan, B. Pharmacology of calcineurin antagonists. Transplant. Proc. 36, 25S-32S (2004).

62. Fryer, J. D. et al. Apolipoprotein E Markedly Facilitates Age-Dependent

Cerebral Amyloid Angiopathy and Spontaneous Hemorrhage in Amyloid Precursor Protein Transgenic Mice. J. Neurosci. 23, 7889-7896 (2003).

63. Antony-Debré I, Paul A, Leite J, Mitchell K, Kim H M, Carvajal L A, Todorova TI, Huang K, Kumar A, Farahat A A, Bartholdy B, Narayanagari S R, Chen J, Ambesi-Impiombato A, Ferrando A A, Mantzaris I, Gavathiotis E, Verma A, Will B, Boykin D W, Wilson W D, Poon G M, Steidl U (2017) Pharmacological inhibition of the transcription factor PU.1 in leukemia. J Clin Invest. pii: 92504.

64. Garcia-Mesa Y, Jay T R, Checkley M A, Luttge B, Dobrowolski C, Valadkhan S, Landreth G E, Karn J, Alvarez Carbonell D (2017) Immortalization of primary microglia: a new platform to study HIV regulation in the central nervous system. J Neurovirol. 23(1): 47-66.

65. Gjoneska E, Pfenning A R, Mathys H, Quon G, Kundaje A, Tsai LH and Kellis M (2015) Conserved epigenomic signals in mice and humans reveal immune basis of Alzheimer's disease. Nat. 518(7539)365-9.

66. Huang K L, Marcora E, Pimenova A A, Di Narzo A F, Kapoor M, Jin S C, Harari O, Bertelsen S, Fairfax B P, Czajkowski J, Chouraki V, Grenier-Boley B, Bellenguez C, Deming Y, McKenzie A, Raj T, Renton A E, Budde J, Smith A, Fitzpatrick A, Bis J C, DeStefano A, Adams H H H, Ikram M A, van der Lee S, Del-Aguila J L, Fernandez M V, Ibañez, International Genomics of Alzheimer's Project, Alzheimer's Disease Neuroimaging Initiative, Sims R, Escott-Price V, Mayeux R, Haines J L, Farrer L A, Pericak-Vance M A, Lambert J C, van Duijn C, Launer L, Seshadri S, Williams J, Amouyel P, Schellenberg G D, Zhang B, Borecki I, Kauwe J S K, Cruchaga C, Hao K and Goate A M (2017) A common haplotype lowers PU.1 expression in myeloid cells and delays onset of Alzheimer's disease. Nat Neurosci. 20(8): 1052-1061.

67. Mathys H, Adaikkan C, Gao F, Young JZ, Manet E, Hemberg M, De Jager P L, Ransohoff R M, Regev A and Tsai L H (2017) Temporal Tracking of Microglia Activation in Neurodegeneration at Single-Cell Resolution. Cell Rep. 21(2)366-380.

68. Munde M, Wang S, Kumar A, Stephens C E, Farahat A A, Boykin D W, Wilson W D, Poon G M (2014) Structure-dependent inhibition of the ETS-family transcription factor PU.1 by novel heterocyclic diamidines. Nucleic Acids Res. 42(2): 1379-90.

69. Rocha S, Campbell K J, Roche K C, Perkins N D (2003) The p53-inhibitor pifithrin-alpha inhibits firefly luciferase activity in vivo and in vitro. BMC Mol Biol. 4:9.

### Sequences

This application throughout describes a variety of amino acid and nucleotide sequences relating to various aspects of the present disclosure, including exemplary and other sequences.

A summary of sequences disclosed herein is as follows in Table 2.

TABLE 2
Exemplary sequences

| SEQ | | |
|---|---|---|
| ID | | |
| NO. | Sequence* | Description |
| 1 | CCAAATAAAAGGA | Lambda-beta motif |
| | AGTGAAACCAA | (LBB) |
| 2 | 5′-CCAAATAAAAGGA | At least 2 LBB |
| | AGTGAAACCAAGCCCA | motifs capable of |
| | AATAAAAGGAAGTGAA | binding PU.l |
| | ACCAAGC-3′ | |
| 3 | X₁GGAAX₂ | Binding motif |
| 4 | CCAAATAAAA | Exemplary X₁ |
| | | sequence |
| 5 | GTGAAACCAA | Exemplary X₂ |
| | | sequence |
| 6 | CCAAATAAAAGGAAG | At least 5 LBB |
| | TGAAACCAAGCCCAA | motifs capable of |
| | ATAAAAGGAAGTGAA | binding PU.l |
| | ACCAAGCCCAAATAA | |
| | AAGGAAGTGAAACCA | |
| | AGCCCAAATAAAAGG | |
| | AAGTGAAACCAAGCC | |
| | CAAATAAAAGGAAGT | |
| | GAAACCAAGC | |
| 7 | TTGGTTTCACTTCCT | |
| | TTTATTTGG | |

*Unless otherwise specified; sequences are shown and described as running 5′ to 3′ for nucleic acid sequences.

### Other Embodiments

Embodiment 1. A recombinant vector comprising a reporter gene under the control of at least two binding motifs capable of binding PU.1 protein.

Embodiment 2. The recombinant vector of embodiment 1, wherein the reporter gene is a gene capable of expressing luciferase.

Embodiment 3. The recombinant vector of embodiment 2, wherein the at least two binding motifs comprise lambda-beta binding motifs (LBB motifs).

Embodiment 4. The recombinant vector of embodiment 3, wherein the at least two binding motifs comprise three LBB motifs.

Embodiment 5. The recombinant vector of embodiment 3, wherein the at least two binding motifs comprise four LBB motifs.

Embodiment 6. The recombinant vector of embodiment 3, wherein the at least two binding motifs comprise five LBB motifs.

Embodiment 7. The recombinant vector of embodiment 3, wherein the at least two binding motifs comprise more than five LBB motifs.

Embodiment 8. The recombinant vector of any one of embodiments 4-7, wherein the LBB motifs are arranged in tandem.

Embodiment 9. The recombinant vector of any one of embodiments 1-7, wherein the at least two binding motifs are within a promoter region.

Embodiment 10. The recombinant vector of any one of embodiments 1-2, wherein the at least two binding motifs are Adenine (A)/Thymine (T) rich.

Embodiment 11. The recombinant vector of embodiment 10, wherein A or T comprise greater than 60% of the nucleotides of the at least two binding motifs.

Embodiment 12. The recombinant vector of embodiment 10, wherein the at least two binding motifs have at least 80% sequence identity to SEQ ID NO: 2.

Embodiment 13. The recombinant vector of embodiment 10, wherein the at least two binding motifs have at least 90% sequence identity to SEQ ID NO: 2.

Embodiment 14. The recombinant vector of embodiment 10, wherein the at least two binding motifs comprise or consist of SEQ ID NO: 2.

Embodiment 15. The recombinant vector of embodiment 10, wherein the at least two binding motifs comprise SEQ ID NO: 3, wherein X₁ is a nucleic acid sequence of 5-15 nucleotides, wherein at least 50% of the nucleotides of X₁ are A or T, and wherein X₂ is a nucleic acid sequence of at least 5 nucleotides, wherein at least 50% of the nucleotides of X₂ are A or T.

Embodiment 16. The recombinant vector of embodiment 15, wherein at least 8 nucleotides of X₁ are A or T.

Embodiment 17. The recombinant vector of embodiment 15, wherein at least 65% of the nucleotides of X₁ are A or T.

Embodiment 18. The recombinant vector of embodiment 15, wherein X₁ comprises SEQ ID NO: 4.

Embodiment 19. The recombinant vector of embodiment 15, wherein at least 6 nucleotides of X₂ are A or T.

Embodiment 20. The recombinant vector of embodiment 15, wherein at least 65% of the nucleotides of X₂ are A or T.

Embodiment 21. The recombinant vector of embodiment 15, wherein X₂ comprises SEQ ID NO: 5.

Embodiment 22. The recombinant vector of any one of embodiments 1-21, wherein the vector is a pGL4.23 luciferase plasmid.

Embodiment 23. The recombinant vector of any one of embodiments 1-21, wherein the vector is a ROSA26-1 plasmid (pROSA26-1).

Embodiment 24. The recombinant vector of embodiment 23, further comprising a Neomycin/G418 resistance gene under the control of a promoter.

Embodiment 25. The recombinant vector of embodiment 1, wherein the vector comprises SEQ ID NO. 6.

Embodiment 26. An isolated cell comprising the recombinant vector of any one of embodiments 1-25.

Embodiment 27. The isolated cell of embodiment 26, wherein the cell is selected from the group comprising: cells from the hematopoietic lineages including both primary from any organism or derived from stem cells, such as a microglial cell, monocytic cell, macrophage cell, T-cells, B-cells, NK-cells, eosinophil cell, neutrophil cell, hematopoietic stem cell, granulocyte cell, dendritic cell, innate lymphoid cell, megakaryocyte cell, myeloid derived suppressor cell, astrocytes, oligodendrocytes, oligodendrocytes precursor cells, and any immortalized lines derived therefrom, including BV2, N9, THP-1, Jurkat cell, Kasumi-1, leukemia cell and their derivatives.

Embodiment 28. The isolated cell of embodiment 27, wherein the cell is a microglial BV2 cell.

Embodiment 29. A method of screening compounds to identify a PU.1 protein inhibitor comprising: (a) exposing an isolated cell from any one of embodiments 26-28 to a compound; (b) incubating the exposed isolated cell of step (a); and (c) quantifying the level of reporter activity of the exposed isolated cell relative to a predetermined level of reporter activity, wherein, a lower level of reporter activity, relative to the predetermined level indicates that the compound is a PU.1 protein inhibitor.

Embodiment 30. The method of screening of embodiment 29, further comprising, excluding false positive results by additional steps (d) and (e), comprising: (d) exposing control cells which constitutively produce reporter to the compound; and (e) quantifying a level of reporter activity of the exposed control cells, wherein, a change of reporter activity of the exposed isolated cell equal to or less than a change of reporter activity of the exposed control cells indicates a false positive.

Embodiment 31. The method of embodiment 30, wherein the reporter gene is a gene capable of expressing luciferase and wherein the cells which constitutively produce reporter are HEK-293 cytomegalovirus (CMV)-Luciferase cells.

Embodiment 32. The method of any one of embodiments 29-31, further comprising, quantifying the level of reporter messenger RNA (mRNA) of the exposed isolated cell.

Embodiment 33. A method of screening compounds to identify a PU.1 inhibitor comprising: (a) contacting a cell with a compound, wherein the cell includes a reporter construct having a reporter gene and a promoter region, wherein the promoter region comprises a PU.1 binding site and quantifying the level of reporter activity of the cell relative to a predetermined level of reporter activity; (b) performing an assay to determine whether the reporter activity detected in the cell is a false positive; (c) if the activity is not a false positive, calculating an effective area under the curve as an indicator of strong activity, wherein the effective area under the curve is calculated by determining a value of percent cell death and a value of percent reduced reporter activity and subtracting the value of percent cell death from the value of percent reduced reporter activity; and (d) determining whether the compound significantly reduces PU.1 dependent gene expression, wherein, a compound that is not a false positive, has an effective area under the curve and reduces PU.1 dependent gene expression is a PU.1 inhibitor.

Embodiment 34. The method of screening of embodiment 33, further comprising, performing the method of embodiment 33 on a plurality of compounds to identify a plurality of PU.1 inhibitors and identifying a structure activity relationship to identify common functional groups in the plurality of PU.1 inhibitors.

Embodiment 35. A library of compounds, wherein each compound in the library comprises a common functional group identified according to the method of embodiment 34.

Embodiment 36. A method of treating a disorder in a subject, comprising: (a) identifying a subject who has, is at risk of having, or is suspected of having a disorder related to PU.1 expression; and (b) administering an effective amount of at least one compound identified as an inhibitor of PU.1 by the method of any one of embodiments 29-32.

Embodiment 37. The method of embodiment 36, wherein the compound of step (b) of embodiment 20, is selected from Table 1.

Embodiment 38. The method of embodiment 36, wherein the disorder is selected from method of treating inflammation in a subject, comprising AD, inflammation, or excessive myelin uptake.

Embodiment 39. The method of embodiment 36, wherein the disorder is AD.

Embodiment 40. The method of any one of embodiments 36-39, wherein the compound is A11:

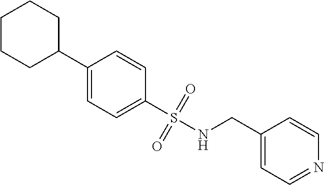 [SMILES: O=S(=O)(NCc1ccncc1)c1ccc(C2CCCCC2)cc1]

and includes pharmaceutically acceptable salts, hydrates, solvates, polymorphs, co-crystals, tautomers, stereoisomers, isotopically labeled derivatives, and prodrugs thereof.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined (i.e., elements that are conjunctively present in some cases and disjunctively present in other cases). Multiple elements listed with “and/or” should be construed in the same fashion (i.e., “one or more” of the elements so conjoined). Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A recombinant vector comprising a reporter gene under the control of at least two binding motifs capable of binding PU.1 protein.

2. The recombinant vector of claim 1, wherein the reporter gene is a gene capable of expressing luciferase.

3. The recombinant vector of claim 2, wherein the at least two binding motifs comprise lambda-beta binding motifs (LBB motifs).

4. The recombinant vector of claim 3, wherein the at least two binding motifs comprise three LBB motifs.

5. The recombinant vector of claim 3, wherein the at least two binding motifs comprise four LBB motifs.

6. The recombinant vector of claim 3, wherein the at least two binding motifs comprise five LBB motifs.

7. The recombinant vector of claim 3, wherein the at least two binding motifs comprise more than five LBB motifs.

8. The recombinant vector of any one of claims 4-7, wherein the LBB motifs are arranged in tandem.

9. The recombinant vector of any one of claims 1-7, wherein the at least two binding motifs are within a promoter region.

10. The recombinant vector of any one of claims 1-2, wherein the at least two binding motifs are Adenine (A)/Thymine (T) rich.

11. The recombinant vector of claim 10, wherein A or T comprise greater than 60% of the nucleotides of the at least two binding motifs.

12. The recombinant vector of claim 10, wherein the at least two binding motifs have at least 80% sequence identity to SEQ ID NO: 2.

13. The recombinant vector of claim 10, wherein the at least two binding motifs have at least 90% sequence identity to SEQ ID NO: 2.

14. The recombinant vector of claim 10, wherein the at least two binding motifs comprise or consist of SEQ ID NO: 2.

15. The recombinant vector of claim 10, wherein the at least two binding motifs comprise SEQ ID NO: 3, wherein X₁ is a nucleic acid sequence of 5-15 nucleotides, wherein at least 50% of the nucleotides of X₁ are A or T, and wherein X₂ is a nucleic acid sequence of at least 5 nucleotides, wherein at least 50% of the nucleotides of X₂ are A or T.

16. The recombinant vector of claim 15, wherein at least 8 nucleotides of X₁ are A or T.

17. The recombinant vector of claim 15, wherein at least 65% of the nucleotides of X₁ are A or T.

18. The recombinant vector of claim 15, wherein X₁ comprises SEQ ID NO: 4.

19. The recombinant vector of claim 15, wherein at least 6 nucleotides of X₂ are A or T.

20. The recombinant vector of claim 15, wherein at least 65% of the nucleotides of X₂ are A or T.

21. The recombinant vector of claim 15, wherein X₂ comprises SEQ ID NO: 5.

22. The recombinant vector of any one of claims 1-21, wherein the vector is a pGL4.23 luciferase plasmid.

23. The recombinant vector of any one of claims 1-21, wherein the vector is a ROSA26-1 plasmid (pROSA26-1).

24. The recombinant vector of claim 23, further comprising a Neomycin/G418 resistance gene under the control of a promoter.

25. The recombinant vector of claim 1, wherein the vector comprises SEQ ID NO. 6.

26. An isolated cell comprising the recombinant vector of any one of claims 1-25.

27. The isolated cell of claim 26, wherein the cell is selected from the group comprising:
cells from the hematopoietic lineages including both primary from any organism or derived from stem cells, such as a microglial cell, monocytic cell, macrophage cell, T-cells, B-cells, NK-cells, eosinophil cell, neutrophil cell, hematopoietic stem cell, granulocyte cell, dendritic cell, innate lymphoid cell, megakaryocyte cell, myeloid derived suppressor cell, astrocytes, oligodendrocytes, oligodendrocytes precursor cells, and any immortalized lines derived therefrom, including BV2, N9, THP-1, Jurkat cell, Kasumi-1, leukemia cell and their derivatives.

28. The isolated cell of claim 27, wherein the cell is a microglial BV2 cell.

29. A method of screening compounds to identify a PU.1 protein inhibitor comprising:
(a) exposing an isolated cell from any one of claims 26-28 to a compound;
(b) incubating the exposed isolated cell of step (a); and
(c) quantifying the level of reporter activity of the exposed isolated cell relative to a predetermined level of reporter activity,
wherein, a lower level of reporter activity, relative to the predetermined level indicates that the compound is a PU.1 protein inhibitor.

30. The method of screening of claim 29, further comprising, excluding false positive results by additional steps (d) and (e), comprising:
(d) exposing control cells which constitutively produce reporter to the compound; and
(e) quantifying a level of reporter activity of the exposed control cells,
wherein, a change of reporter activity of the exposed isolated cell equal to or less than a change of reporter activity of the exposed control cells indicates a false positive.

31. The method of claim 30, wherein the reporter gene is a gene capable of expressing luciferase and wherein the cells which constitutively produce reporter are HEK-293 cytomegalovirus (CMV)-Luciferase cells.

32. The method of any one of claims 29-31, further comprising, quantifying the level of reporter messenger RNA (mRNA) of the exposed isolated cell.

33. A method of screening compounds to identify a PU.1 inhibitor comprising:
(a) contacting a cell with a compound, wherein the cell includes a reporter construct having a reporter gene and a promoter region, wherein the promoter region comprises a PU.1 binding site and quantifying the level of reporter activity of the cell relative to a predetermined level of reporter activity;
(b) performing an assay to determine whether the reporter activity detected in the cell is a false positive;
(c) if the activity is not a false positive, calculating an effective area under the curve as an indicator of strong activity, wherein the effective area under the curve is calculated by determining a value of percent cell death and a value of percent reduced reporter activity and subtracting the value of percent cell death from the value of percent reduced reporter activity; and
(d) determining whether the compound significantly reduces PU.1 dependent gene expression,
wherein, a compound that is not a false positive, has an effective area under the curve and reduces PU.1 dependent gene expression is a PU.1 inhibitor.

34. The method of screening of claim 33, further comprising, performing the method of claim 33 on a plurality of compounds to identify a plurality of PU.1 inhibitors and identifying a structure activity relationship to identify common functional groups in the plurality of PU.1 inhibitors.

35. A library of compounds, wherein each compound in the library comprises a common functional group identified according to the method of claim 34.

36. A pharmaceutical composition comprising:
a compound of Formula I: 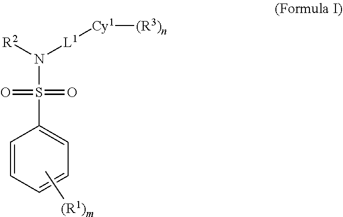
or a pharmaceutically acceptable salt, solvate, hydrate, polymorph, co-crystal, tautomer, stereoisomer, isotopically labeled derivative, or prodrug thereof; and
one or more pharmaceutically acceptable excipients;
wherein:
each instance of R¹ is independently halogen, substituted or unsubstituted alkyl, —OR^{a}, substituted or unsubstituted carbocyclyl, substituted or unsubstituted heterocyclyl, or substituted or unsubstituted heteroaryl;
or two instances of R¹ are joined with the intervening atoms to form substituted or unsubstituted heterocyclyl or substituted or unsubstituted heteroaryl, and each of the remaining instances of R¹, if present, is independently halogen, substituted or unsubstituted alkyl, or —OR^{a};
each instance of R^{a} is independently substituted or unsubstituted alkyl, substituted or unsubstituted carbocyclyl, or substituted or unsubstituted aryl;
m is 1, 2, or 3;
R² is hydrogen or —C(═O)OR^{a};
L¹ is a single bond or substituted or unsubstituted methylene;
Cy^{l} is aryl or heteroaryl;
each instance of R³ is independently halogen, substituted or unsubstituted alkyl, —OR^{a}, —N(R^{a})₂, —C(═O)R^{a}, —C(═O)OR^{a}, or —S(═O)₂R^{a};
or Cy¹ is a single bond, and R² and one instance of R³ are joined with the intervening atoms to form substituted or unsubstituted heterocyclyl or substituted or unsubstituted heteroaryl, and each of the remaining instances of R³, if present, is independently halogen, substituted or unsubstituted alkyl, —OR^{a}, —N(R^{a})₂, —C(═O)R^{a}, —C(═O)OR^{a}, or —S(═O)₂R^{a}; and
n is 0, 1, 2, or 3.

37. The pharmaceutical composition of claim 36, wherein at least one instance of R¹ is substituted or unsubstituted carbocyclyl.

38. The pharmaceutical composition of claim 36 or 37, wherein R² is hydrogen.

39. The pharmaceutical composition of any one of claims 36-38, wherein L¹ is unsubstituted methylene.

40. The pharmaceutical composition of any one of claims 36-39, wherein Cy¹ is pyridyl.

41. The pharmaceutical composition of claim 36, wherein the compound is of the formula: 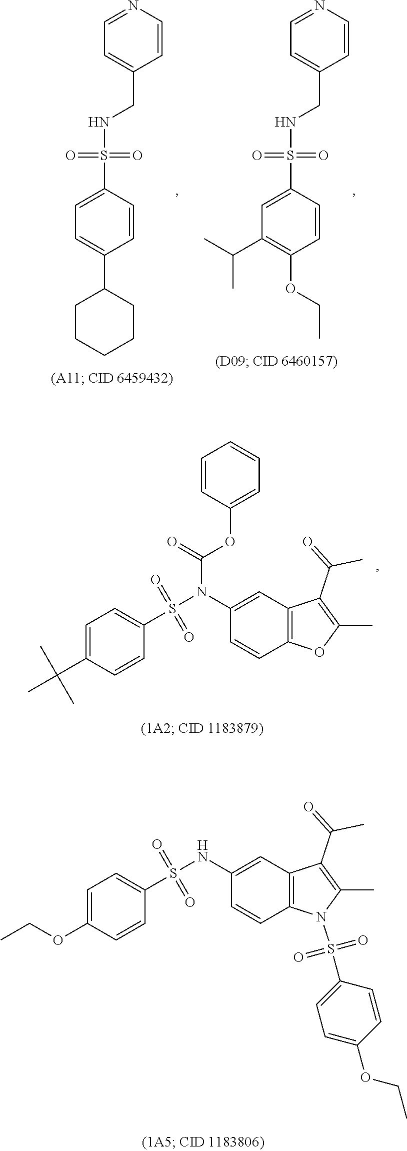 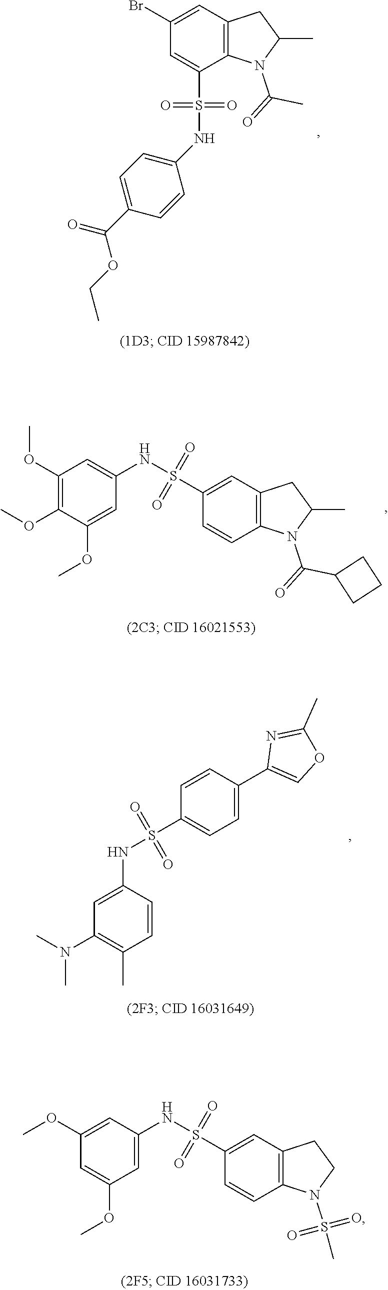 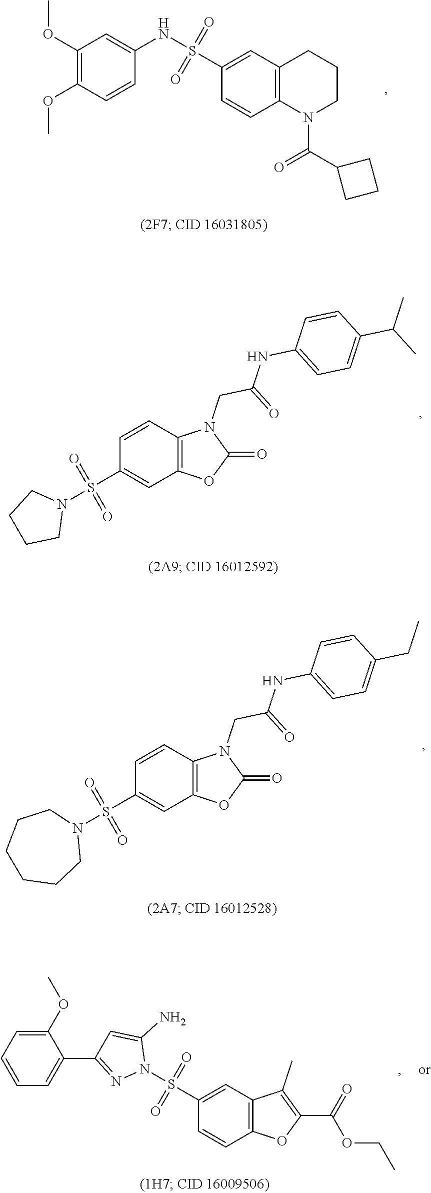 [SMILES: COc1ccc(NS(=O)(=O)c2ccc3c(c2)CCCN3C(=O)C2CCC2)cc1OC ; CC(C)c1ccc(NC(=O)Cn2c(=O)oc3cc(S(=O)(=O)N4CCCC4)ccc32)cc1 ; CCc1ccc(NC(=O)Cn2c(=O)oc3cc(S(=O)(=O)N4CCCCCC4)ccc32)cc1 ; CCOC(=O)c1oc2ccc(S(=O)(=O)n3nc(-c4ccccc4OC)cc3N)cc2c1C] 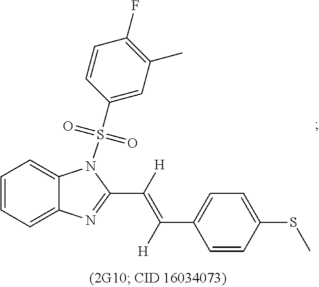 [SMILES: CSc1ccc(/C=C/c2nc3ccccc3n2S(=O)(=O)c2ccc(F)c(C)c2)cc1]
or a pharmaceutically acceptable salt, solvate, hydrate, polymorph, co-crystal, tautomer, stereoisomer, isotopically labeled derivative, or prodrug thereof.

42. A pharmaceutical composition comprising:
a compound of Formula II: 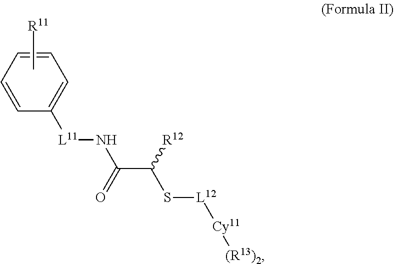
or a pharmaceutically acceptable salt, solvate, hydrate, polymorph, co-crystal, tautomer, stereoisomer, isotopically labeled derivative, or prodrug thereof; and
one or more pharmaceutically acceptable excipients;
wherein:
R¹¹ is hydrogen, halogen, or substituted or unsubstituted alkyl;
L¹¹ is a single bond, substituted or unsubstituted methylene, or substituted or unsubstituted ethylene;
R¹² is hydrogen or substituted or unsubstituted alkyl;
L¹² is a single bond or substituted or unsubstituted methylene;
Cy^{1l} is heteroaryl or heterocyclyl; and
each instance of R¹³ is independently substituted or unsubstituted alkyl, substituted or unsubstituted aryl, or oxo.

43. The pharmaceutical composition of claim 42, wherein R¹¹ is hydrogen, halogen, or unsubstituted C₁₋₆ alkyl.

44. The pharmaceutical composition of claim 42 or 43, wherein L¹¹ is a single bond, unsubstituted methylene, or unsubstituted ethylene.

45. The pharmaceutical composition of any one of claims 42-44, wherein R¹² is hydrogen or unsubstituted C₁₋₆ alkyl.

46. The pharmaceutical composition of any one of claims 42-45, wherein L¹² is a single bond or unsubstituted methylene.

47. The pharmaceutical composition of any one of claims 42-46, wherein Cy^{1l} is oxazolyl or

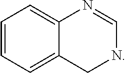

48. The pharmaceutical composition of any one of claims 42-47, wherein each instance of R¹³ is independently unsubstituted C₁₋₆ alkyl, substituted or unsubstituted phenyl, or oxo.

49. The pharmaceutical composition of claim 42, wherein the compound is of the formula: 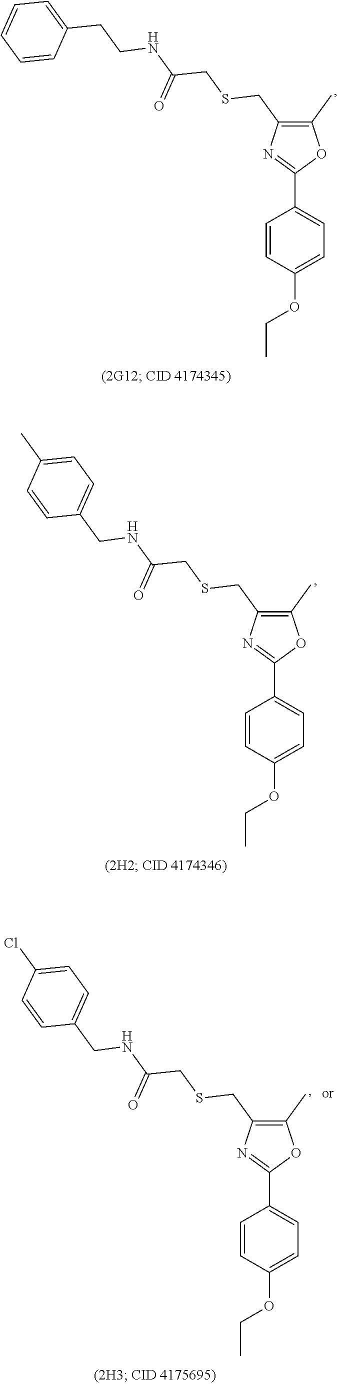 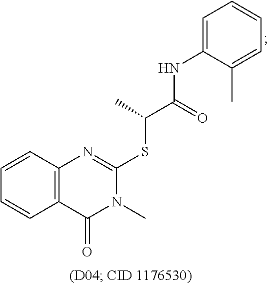 [SMILES: Cc1ccccc1NC(=O)[C@@H](C)Sc1nc2ccccc2c(=O)n1C]
or a pharmaceutically acceptable salt, solvate, hydrate, polymorph, co-crystal, tautomer, stereoisomer, isotopically labeled derivative, or prodrug thereof.

50. A pharmaceutical composition comprising:
a compound of Formula III: 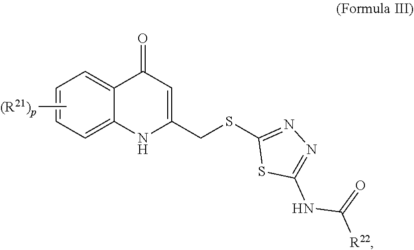
or a pharmaceutically acceptable salt, solvate, hydrate, polymorph, co-crystal, tautomer, stereoisomer, isotopically labeled derivative, or prodrug thereof; and
one or more pharmaceutically acceptable excipients;
wherein:
each instance of R²¹ is substituted or unsubstituted alkyl;
p is 1 or 2; and
R²² is substituted or unsubstituted alkyl or substituted or unsubstituted aryl.

51. The pharmaceutical composition of claim 50, wherein each instance of R²¹ is unsubstituted C₁₋₆ alkyl.

52. The pharmaceutical composition of claim 50 or 51, wherein R²² is unsubstituted C₁₋₆ alkyl or substituted or unsubstituted phenyl.

53. The pharmaceutical composition of claim 50, wherein the compound is of the formula: 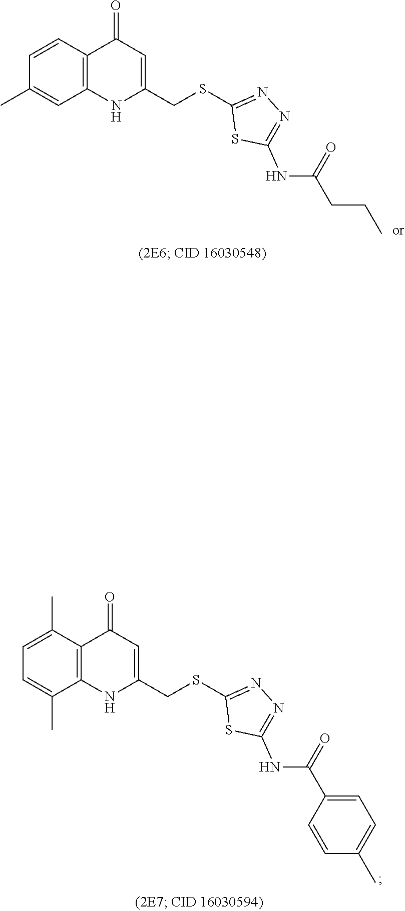 [SMILES: CCCC(=O)Nc1nnc(SCc2cc(=O)c3ccc(C)cc3[nH]2)s1 ; Cc1ccc(C(=O)Nc2nnc(SCc3cc(=O)c4c(C)ccc(C)c4[nH]3)s2)cc1]
or a pharmaceutically acceptable salt, solvate, hydrate, polymorph, co-crystal, tautomer, stereoisomer, isotopically labeled derivative, or prodrug thereof.

54. A pharmaceutical composition comprising:
a compound of Formula IV: 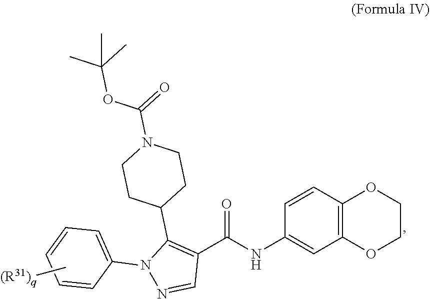
or a pharmaceutically acceptable salt, solvate, hydrate, polymorph, co-crystal, tautomer, stereoisomer, isotopically labeled derivative, or prodrug thereof; and
one or more pharmaceutically acceptable excipients;
wherein:
each instance of R³¹ is independently halogen or substituted or unsubstituted alkyl;
and
q is0,1,or2.

55. The pharmaceutical composition of claim 54, wherein each instance of R³¹ is independently halogen or unsubstituted C₁₋₆ alkyl.

56. The pharmaceutical composition of claim 54, wherein the compound is of the formula: 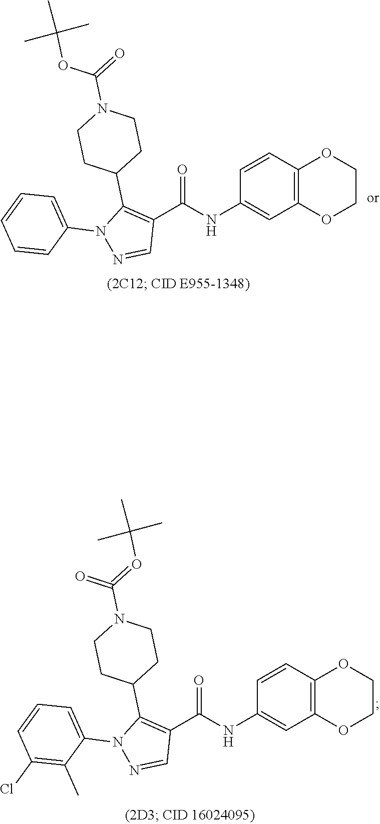
or a pharmaceutically acceptable salt, solvate, hydrate, polymorph, co-crystal, tautomer, stereoisomer, isotopically labeled derivative, or prodrug thereof.

57. A pharmaceutical composition comprising:
a compound of the formula: 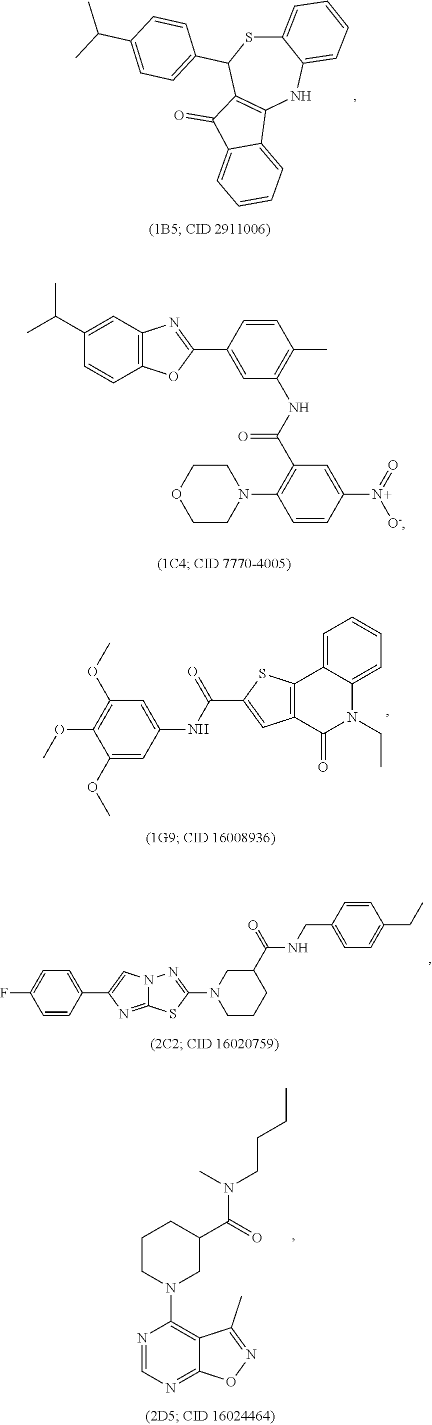 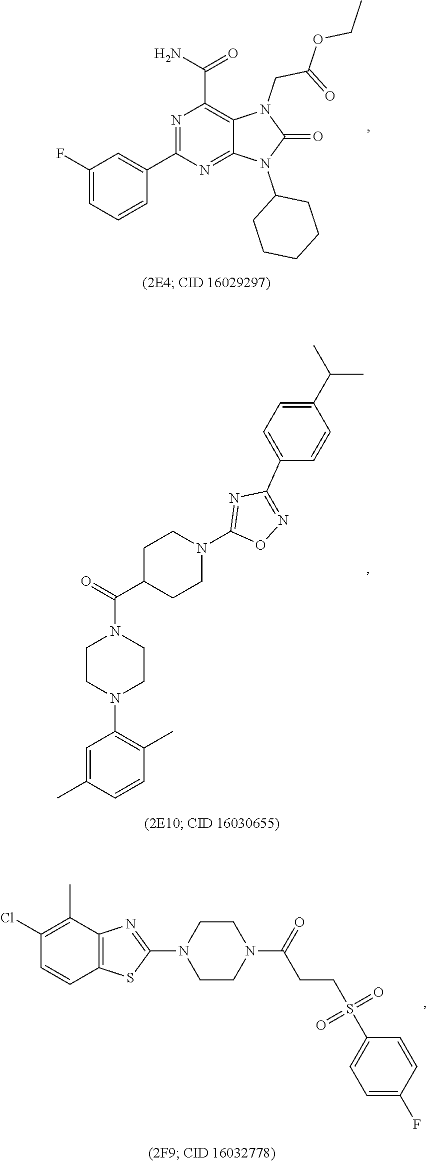 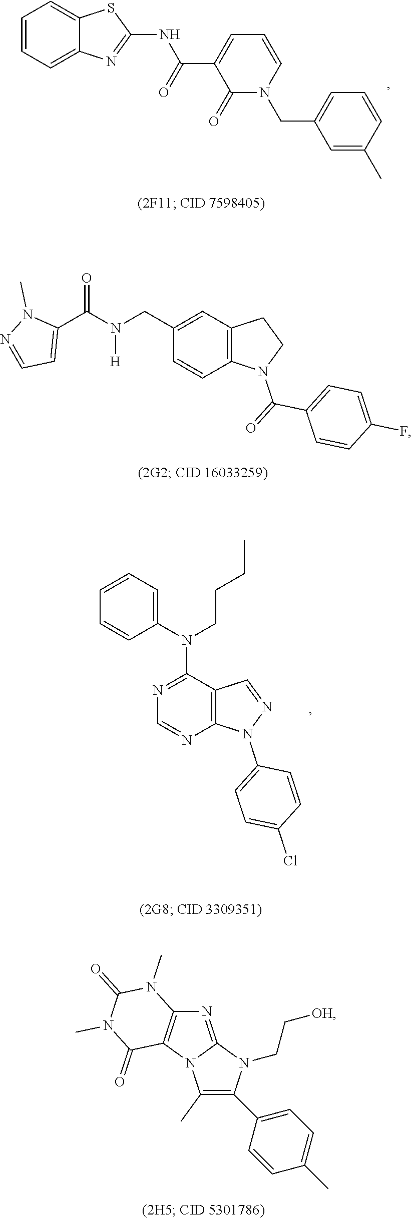 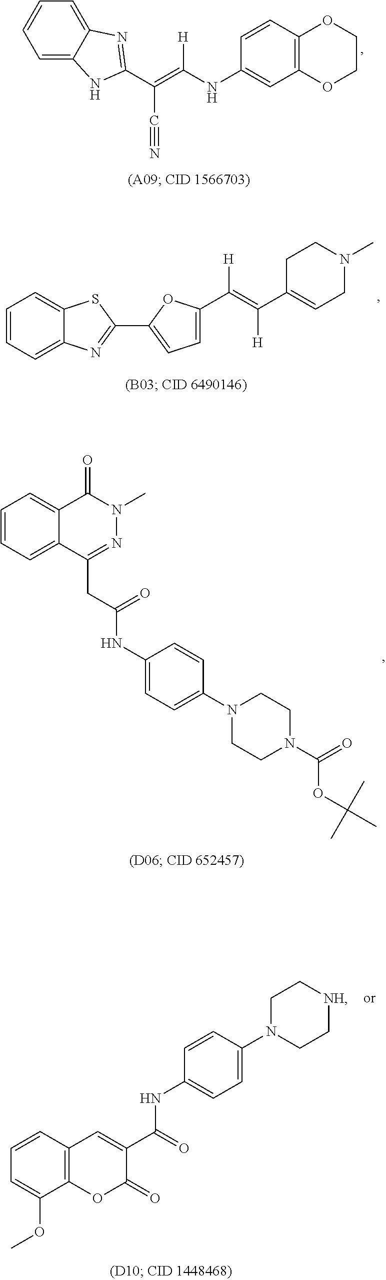 [SMILES: N#C/C(=C\Nc1ccc2c(c1)OCCO2)c1nc2ccccc2[nH]1 ; CN1CC=C(/C=C/c2ccc(-c3nc4ccccc4s3)o2)CC1 ; Cn1nc(CC(=O)Nc2ccc(N3CCN(C(=O)OC(C)(C)C)CC3)cc2)c2ccccc2c1=O ; COc1cccc2cc(C(=O)Nc3ccc(N4CCNCC4)cc3)c(=O)oc12 ; "or"] 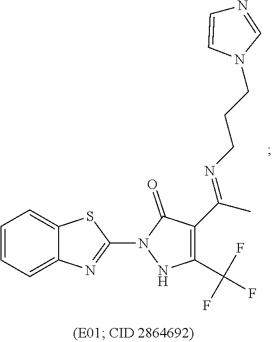 [SMILES: C/C(=N\CCCn1ccnc1)c1c(C(F)(F)F)[nH]n(-c2nc3ccccc3s2)c1=O]
or a pharmaceutically acceptable salt, solvate, hydrate, polymorph, co-crystal, tautomer, stereoisomer, isotopically labeled derivative, or prodrug thereof; and
one or more pharmaceutically acceptable excipients.

58. The pharmaceutical composition of any one of claims 36-57, wherein at least one pharmaceutically acceptable excipient is
a) not water;
b) sterile;
c) in a solid or gel formulation;
d) is in a therapeutically effective amount in a formulation;
e) is in a tablet or capsule formulation; or
f) is in an injectable formulation and or device.

59. A method of treating a disorder in a subject, comprising:
(a) identifying a subject who has, is at risk of having, or is suspected of having a disorder related to PU.1 expression; and
(b) administering an effective amount of at least one compound identified as an inhibitor of PU.1 by the method of any one of claims 29-32 or an effective amount of at least one pharmaceutical composition of any one of claims 36-58.

60. The method of claim 59, wherein the compound of step (b) of claim 20, is selected from Table 1.

61. The method of claim 59, wherein the disorder is selected from method of treating inflammation in a subject, comprising AD, inflammation, or excessive myelin uptake.

62. The method of claim 59, wherein the disorder is AD.

63. The method of any one of claims 59-62, wherein the compound is A11: 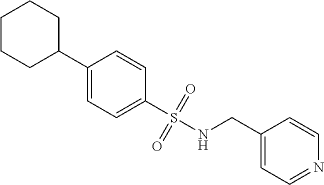 [SMILES: O=S(=O)(NCc1ccncc1)c1ccc(C2CCCCC2)cc1]
and pharmaceutically acceptable salts, hydrates, solvates, polymorphs, co-crystals, tautomers, stereoisomers, isotopically labeled derivatives, and prodrugs thereof.

64. The method of any one of claims 59-62, wherein the compound is of Formula V: 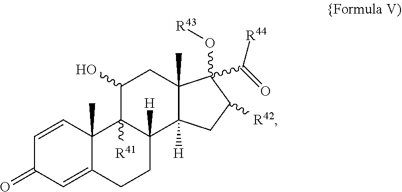
or a pharmaceutically acceptable salt, solvate, hydrate, polymorph, co-crystal, tautomer, stereoisomer, isotopically labeled derivative, or prodrug thereof, wherein:
R⁴¹ is hydrogen or halogen;
R⁴² is hydrogen or substituted or unsubstituted alkyl;
R⁴³ is hydrogen, —C(═O)R^{e}, or —C(═O)OR^{e};
each instance of R^{e} is independently substituted or unsubstituted alkyl or substituted or unsubstituted heteroaryl; and
R⁴⁴ is substituted or unsubstituted alkyl or —OR^{e}.

65. The method of claim 64, wherein R⁴² is hydrogen or unsubstituted C₁₋₆ alkyl.

66. The method of claim 64 or 65, wherein R⁴⁴ is C₁₋₆ alkyl substituted with at least one halogen or at least one —OH, or is —OR^{e}.

67. The method of any one of claims 64-66, wherein each instance of R^{e} is independently unsubstituted C₁₋₆ alkyl, C₁₋₆ alkyl substituted with at least one halogen, or substituted or unsubstituted furanyl.

68. The method of claim 64, wherein the compound is of the formula: 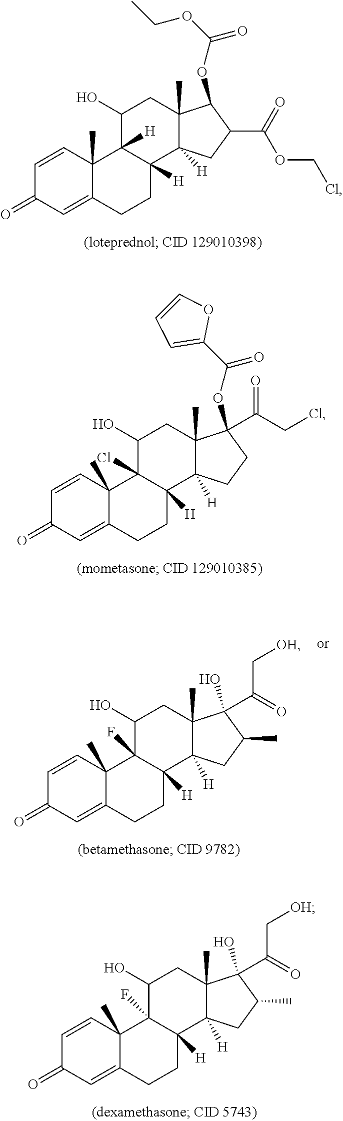
or a pharmaceutically acceptable salt, solvate, hydrate, polymorph, co-crystal, tautomer, stereoisomer, isotopically labeled derivative, or prodrug thereof.

69. The method of any one of claims 59-62, wherein the compound is of the formula: 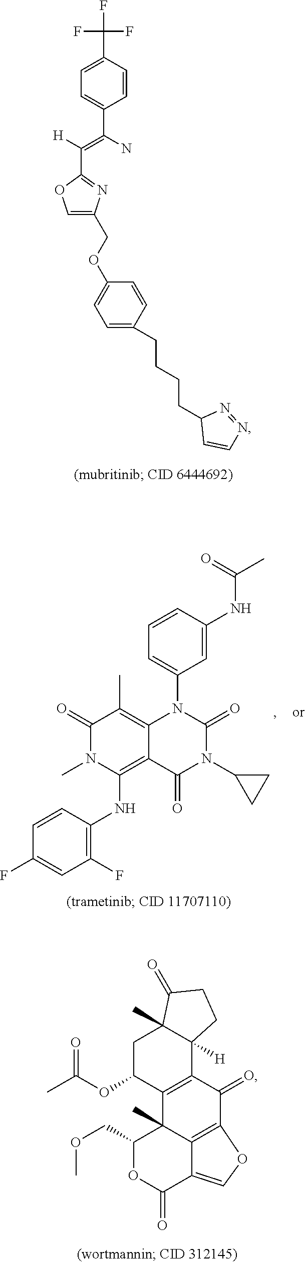 [SMILES: N/C(=C\c1nc(COc2ccc(CCCCC3C=CN=N3)cc2)co1)c1ccc(C(F)(F)F)cc1 ; CC(=O)Nc1cccc(-n2c(=O)n(C3CC3)c(=O)c3c(Nc4ccc(F)cc4F)n(C)c(=O)c(C)c32)c1 ; COC[C@H]1OC(=O)c2coc3c2[C@@]1(C)C1=C(C3=O)[C@@H]2CCC(=O)[C@@]2(C)C[C@H]1OC(C)=O]
or a pharmaceutically acceptable salt, solvate, hydrate, polymorph, co-crystal, tautomer, stereoisomer, isotopically labeled derivative, or prodrug thereof.

70. The method of any one of claims 59-62, wherein the compound is not a compound of Formula V: 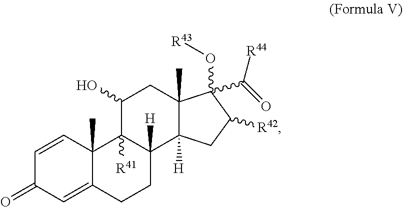
or a pharmaceutically acceptable salt, solvate, hydrate, polymorph, co-crystal, tautomer, stereoisomer, isotopically labeled derivative, or prodrug thereof, wherein:
R⁴¹ is hydrogen or halogen;
R⁴² is hydrogen or substituted or unsubstituted alkyl;
R⁴³ is hydrogen, —C(═O)R^{e}, or —C(═O)OR^{e};
each instance of R^{e} is independently substituted or unsubstituted alkyl or substituted or unsubstituted heteroaryl; and
R⁴⁴ is substituted or unsubstituted alkyl or —OR^{e}.

71. The method of claim 64, wherein the compound is not a compound of the formula:

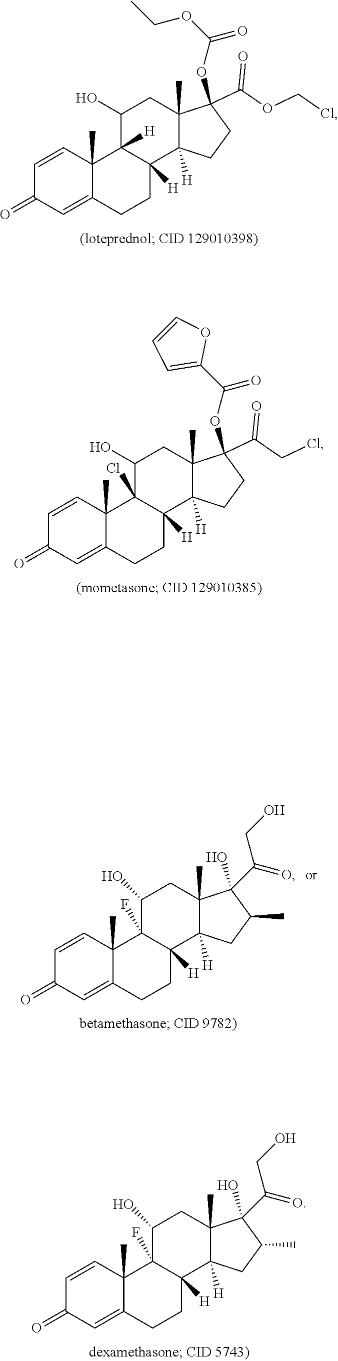

72. The method of any one of claims 59-62, wherein the compound is not the compound of the formula: 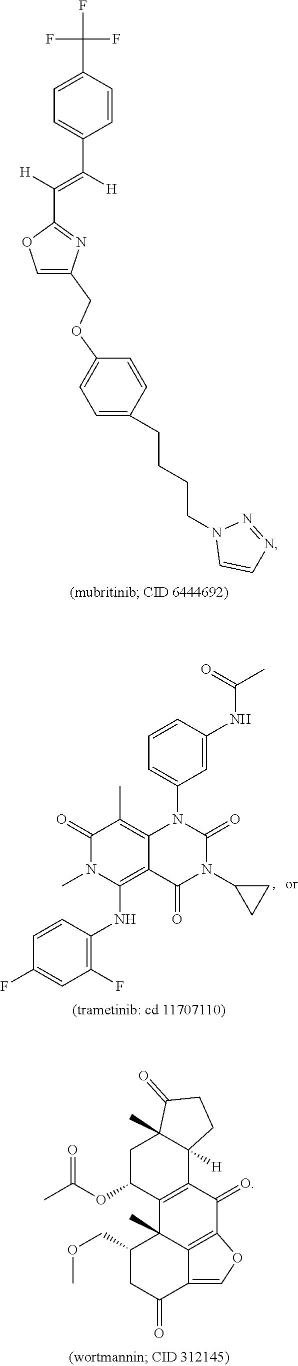 [SMILES: FC(F)(F)c1ccc(/C=C/c2nc(COc3ccc(CCCCn4ccnn4)cc3)co2)cc1 ; CC(=O)Nc1cccc(-n2c(=O)n(C3CC3)c(=O)c3c(Nc4ccc(F)cc4F)n(C)c(=O)c(C)c32)c1 ; COC[C@H]1CC(=O)c2coc3c2[C@@]1(C)C1=C(C3=O)[C@@H]2CCC(=O)[C@@]2(C)C[C@H]1OC(C)=O]
